(19) United States
(12) Patent Application Publication
(10) Pub. No.: US 2006/0160865 A1
(43) Pub. Date: Jul. 20, 2006

(54) Pyridinyl carbamates

(75) Inventors: Johannes Cornelis de Jong, Bagsvaerd (DK); Poul Jacobsen, Slangerup (DK); Soren Ebdrup, Roskilde (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/301,224

(22) Filed: Dec. 12, 2005

Related U.S. Application Data
(63) Continuation of application No. PCT/DK04/00391, filed on Jun. 8, 2004.

(30) Foreign Application Priority Data
Jun. 12, 2003 (DK) PA 2003 00879

(51) Int. Cl. C07D 213/63; A61K 31/4412

(52) U.S. Cl. 514/346; 546/291

(57) ABSTRACT

Novel substituted pyridinyl carbamates, pharmaceutical compositions comprising them and use thereof in the treatment and/or prevention of diseases and disorders related to hormone sensitive lipase. More particularly, the compounds are useful for the treatment and/or prevention of diseases and disorders in which modulation of the activity of hormone sensitive lipase is beneficial.

### CROSS-REFERENCE TO RELATED APPLICATIONS

This application is a continuation of International Application No.: PCT/DK2004/000391, filed Jun. 8, 2004, which claims priority to Danish Patent Application No.: PA 2003 00879, filed Jun. 12, 2003, and U.S. Patent Application No. 60/478,526 filed Jun. 13, 2003.

### FIELD OF THE INVENTION

The present invention relates to novel substituted pyridinyl carbamates, to pharmaceutical compositions comprising these compounds, to the use of these compounds as pharmaceutical compositions, and to methods of treatment employing these compounds and compositions. The present compounds show inhibition of hormone sensitive lipase. As a result, the compounds are useful for the treatment and/or prevention of diseases and disorders related to hormone sensitive lipase.

### BACKGROUND OF THE INVENTION

The overall energy homeostasis of a mammalian system requires a high degree of regulation to ensure the availability of the appropriate substrate at the appropriate time. Plasma glucose levels rise during the post-prandial state, to return to pre-prandial levels within 2-3 hours. During these 2-3 hours, insulin promotes glucose uptake by skeletal muscle and adipose tissue and decreases the release of free fatty acids (FFA) from adipocytes, to ensure that the two substrates do not compete with each other. When plasma glucose levels fall, an elevation in plasma FFA is necessary to switch from glucose to fat utilization by the various tissues.

In individuals with insulin resistance, FFA levels do not fall in response to insulin, as they do in normal individuals, preventing the normal utilization of glucose by skeletal muscle, adipose and liver. Furthermore, there is a negative correlation between insulin sensitivity and plasma FFA levels.

Hormone-sensitive lipase (HSL) is an enzyme, expressed in adipose tissue, macrophages, muscle, adrenal, testis and islets (Kraemer and Shen, J. Lipid Res. 2002, 43, 1585-1594). In the adipocytes HSL catalyses the conversion of triglycerides to glycerol and fatty acids. It is through the regulation of this enzyme that the levels of circulating FFA are modulated. Insulin leads to the inactivation of HSL with a subsequent fall in plasma FFA levels during the post-prandial state, followed by the activation of the enzyme when the insulin concentration falls and catecholamines rise during the post-absorptive period. The activation of HSL leads to an increase in plasma FFA, as they become the main source of energy during fasting.

The activation-inactivation of HSL is primarily mediated through the cAMP-protein kinase A and AMP-dependent kinase pathways. There are compounds like nicotinic acid and its derivatives, that decrease the activation of HSL via these pathways and cause a decrease in lipolysis that leads to a reduction in the FFA levels. These drugs have a beneficial effect in the utilization of glucose and in the normalization of the excess triglyceride synthesis seen in patients with elevated FFA. However, since these pathways are used by other processes in the body, these drugs have severe side effects.

Carbamates similar to the compounds of the present invention have previously been prepared. Ross Kelly et al. (Org. Lett. 24(1), 2001, 3895-3898) disclose the following compound and its use in a chemically powered molecular motor:

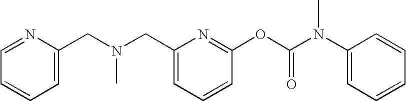

Several publications disclose the preparation and use of HSL inhibitors (WO 01/87843, WO 01/17981, WO 01/66531, WO 01/83497, and WO 01/26664). However, the structures of these compounds are very different from that of the present compounds. Thus, none of the HSL inhibitors disclosed in these publications contain pyridinyl and carbamate substructures as in the compounds of the present invention.

We have found potent pyridinyl carbamate compounds that specifically inhibit the lipolytic activity of HSL and which may be expected to decrease plasma FFA levels. These compounds can be used to treat disorders where a decreased level of plasma FFA is desired, such as insulin resistance, syndrome X, dyslipidemia, abnormalities of lipoprotein metabolism.

One aspect of the present invention is to provide compounds and pharmaceutical compositions that inhibit the lipolytic activity of HSL. A further aspect is to provide compounds which have good pharmaceutical properties such as solubility, bioavailability, specificity etc.

### DEFINITIONS

The following is a detailed definition of the terms used to describe the compounds of the invention.

The term “halogen” in the present context designates an atom selected from the group consisting of F, Cl, Br and I.

The term “C₁₋₆-alkyl” in the present context designates a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, iso-pentyl, neopentyl, tert-pentyl, n-hexyl, isohexyl and the like.

The term “C₂₋₆-alkyl” in the present context designates a saturated, branched or straight hydrocarbon group having from 2 to 6 carbon atoms. Representative examples include, but are not limited to, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁₋₆-alkoxy” in the present context designates a group —O—C₁₋₆-alkyl wherein C₁₋₆-alkyl is as defined above. Representative examples include, but are not limited to, methoxy, ethoxy, n-propoxy, isopropoxy, butoxy, isobutoxy, sec-butoxy, tert-butoxy, n-pentoxy, isopentoxy, neopentoxy, tert-pentoxy, n-hexoxy, isohexoxy and the like.

The term “C₂₋₆-alkenyl” as used herein, represent an olefinically unsaturated branched or straight hydrocarbon group having from 2 to 6 carbon atoms and at least one double bond. Examples of such groups include, but are not limited to, vinyl, 1-propenyl, 2-propenyl, allyl, iso-propenyl, 1,3-butadienyl, 1-butenyl, hexenyl, pentenyl and the like.

The term “C₃₋₁₀-cycloalkyl” as used herein represents a saturated mono-, bi-, tri- or spirocarbocyclic group having from 3 to 10 carbon atoms. Representative examples are cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “C₃₋₈-heterocyclyl” as used herein represents a saturated 3 to 8 membered ring containing one or more heteroatoms selected from nitrogen, oxygen and sulfur. Representative examples are pyrrolidyl, piperidyl, piperazinyl, morpholinyl, thiomorpholinyl, aziridinyl, tetrahydrofuranyl and the like.

The term “aryl” as used herein represents a carbocyclic aromatic ring system being either monocyclic, bicyclic, or polycyclic, such as phenyl, biphenyl, naphthyl, anthracenyl, phenanthrenyl, fluorenyl, indenyl, pentalenyl, azulenyl, biphenylenyl and the like. Aryl is also intended to include the partially hydrogenated derivatives of the carbocyclic aromatic systems enumerated above. Non-limiting examples of such partially hydrogenated derivatives are 1,2,3,4-tetrahydronaphthyl, 1,4-dihydronaphthyl and the like.

The term “heteroaryl” as used herein represents a heterocyclic aromatic ring system containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, 3,4-dihydroisoquinolinyl, pyrrolinyl, pyrazolinyl, indolinyl, oxazolidinyl, oxazolinyl, oxazepinyl and the like.

The term “perhalomethyl” as used herein designates a methyl moiety substituted with three halogen atoms. Non-limiting examples of perhalomethyl are CF₃, CCl₃, and CF₂Cl.

The term “perhalomethoxy” as used herein designates a perhalomethyl linked via an oxygen atom, e.g. —O—CF₃, —O—CCl₃, and —O—CF₂Cl

The term “ring system” as used herein includes aromatic as well as non-aromatic ring moieties, which may be monocyclic, bicyclic or polycyclic, and they encompass moieties with zero, one or more hetereatoms selected from nitrogen, oxygen and sulphur. Non-limiting examples of such ring systems are aryl, C₃₋₈-heterocyclyl and heteroaryl.

The term “heterocyclic system” as used herein includes aromatic as well as non-aromatic ring moieties, which may be monocyclic, bicyclic or polycyclic, and containing in their ring structure one or more heteroatoms selected from nitrogen, oxygen and sulfur. Non-limiting examples of such heterocyclic systems are C₃₋₈-heterocyclyl and heteroaryl.

Certain of the above defined terms may occur more than once in the structural formulae, and upon such occurrence each term shall be defined independently of the other.

The term “optionally substituted” as used herein means that the groups in question are either unsubstituted or substituted with one or more of the substituents specified. When the groups in question are substituted with more than one substituent the substituents may be the same or different.

The terms “disease”, “condition” and “disorder” as used herein are used interchangeably to specifiy a state of a patient which is not the normal physiological state of man.

The term “treatment” as used herein means the management and care of a patient having developed a disease, condition or disorder, as well as the management and care of an individual at risk of developing the disease, condition or disorder prior to the clinical onset of said disease, condition or disorder. The purpose of treatment is to combat the disease, condition or disorder, as well as to to combat the development of the disease, condition or disorder. Treatment includes the administration of the active compounds to prevent or delay the onset of the symptoms or complications and to eliminate or control the disease, condition or disorder as well as to alleviate the symptoms or complications associated with the disease, condition or disorder.

The term “effective amount” as used herein means a dosage which is sufficient in order for the treatment of the patient to be effective compared with no treatment.

The term “modulate” as used herein means to influence, i.e. to modulate a parameter means to influence that parameter in a desired way. Examples are to modulate insulin secretion from beta cells and to modulate the plasma level of free fatty acids.

The term “medicament” as used herein means a pharmaceutical composition suitable for administration of the pharmaceutically active compound to a patient.

The term “pharmaceutically acceptable” as used herein means suited for normal pharmaceutical applications, i.e. giving rise to no adverse events in patients etc.

### DESCRIPTION OF THE INVENTION

In one aspect the present invention relates to a compound of the general formula (I): 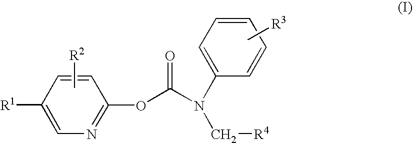
wherein
R¹ and R² are independently selected from hydrogen, hydroxy, sulfanyl, amino, amide, urea, thiourea, benzamide, thioamide, halogen, sulfo, C₁₋₆-alkyl, C₁₋₆alkoxy, C₂₋₆-alkenyl, aryl, heteroaryl, C₃₋₈-heterocyclyl and C₃₋₁₀-cycloalkyl, wherein each of hydroxy, sulfanyl, amino, amide, urea, thiourea, benzamide, thioamide, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, thioxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R³, and R⁴ are independently selected from hydrogen, hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
with the proviso that said compound is not

2,2-Dimethyl-N-[6-(methyl-phenyl-carbamoyloxy)-pyridin-3-yl]-succinamic acid, 3,3-Dimethyl-4-[6-(methyl-phenyl-carbamoyloxy)-pyridin-3-ylcarbamoyl]-butyric acid, Methyl-phenyl-carbamic acid pyridin-2-yl ester, Methyl-phenyl-carbamic acid 5-chloro-pyridin-2-yl ester, Methyl-phenyl-carbamic acid 5-trifluoromethyl-pyridin-2-yl ester, Methyl-phenyl-carbamic acid 3-chloro-5-trifluoromethyl-pyridin-2-yl ester, Methyl-phenyl-carbamic acid 5-benzoylamino-pyridin-2-yl ester, Methyl-phenyl-carbamic acid 5-(cyclohexanecarbonyl-amino)-pyridin-2-yl ester, Methyl-phenyl-carbamic acid 4,4-dimethyl-2,6-dioxo-3,4,5,6-tetrahydro-2H-[1,3′]bipyridinyl-6′-yl ester, Methyl-phenyl-carbamic acid 5-(2,2-dimethyl-propionylamino)-pyridin-2-yl ester, Methyl-phenyl-carbamic acid 5-(2-cyclohexyl-acetylamino)-pyridin-2-yl ester, Methyl-phenyl-carbamic acid 5-(3,3-dimethyl-butyrylamino)-pyridin-2-yl ester, Methyl-phenyl-carbamic acid 5-[(pyridine-2-carbonyl)-amino]-pyridin-2-yl ester, Methyl-phenyl-carbamic acid 5-[(6-chloropyridine-3-carbonyl)-amino]-pyridin-2-yl ester, Methyl-phenyl-carbamic acid 5-(2,2-dimethyl-propylcarbamoyl)-pyridin-2-yl ester, Methyl-phenyl-carbamic acid 2,6-dioxo-3,4,5,6-tetrahydro-2H-[1,3′]bipyridinyl-6′-yl ester, Methyl-phenyl-carbamic acid 5-(2,5-dioxo-pyrrolidin-1-yl)-pyridin-2-yl ester, Methyl-phenyl-carbamic acid 5-(4-trifluoromethyl-benzoylamino)-pyridin-2-yl ester, Methyl-phenyl-carbamic acid 5-(4-chloro-benzoylamino)-pyridin-2-yl ester, Methyl-phenyl-carbamic acid 5-(4-methoxy-benzoylamino)-pyridin-2-yl ester, Methyl-phenyl-carbamic acid 4,4-dimethyl-3,4,5,6-tetrahydro-2H-[1,3′]bipyridinyl-6′-yl ester, Methyl-phenyl-carbamic acid 5-amino-pyridin-2-yl ester, Methyl-phenyl-carbamic acid 5-benzenesulfonylamino-pyridin-2-yl ester, Methyl-phenyl-carbamic acid 5-(3,3-dimethyl-2,5-dioxo-pyrrolidin-1-yl)-pyridin-2-yl ester, Methyl-phenyl-carbamic acid 5-[3,3-dimethyl-5-(4-methyl-piperazin-1-yl)-5-oxo-pentanoylamino]pyridin-2-yl ester, Methyl-phenyl-carbamic acid 5-[3,3-dimethyl-4-(pyridin-3-ylcarbamoyl)-butyrylamino]-pyridin-2-yl ester, Methyl-phenyl-carbamic acid 5-(3,3-dimethyl-5-morpholin-4-yl-5-oxo-pentanoylamino)-pyridin-2-yl ester, Methyl-phenyl-carbamic acid 5-[4-(2-dimethylamino-ethylcarbamoyl)-3,3-dimethyl-butyrylamino]-pyridin-2-yl ester; N-Methyl-N-phenylcarbamic acid 5-nitro-3-trifluoromethylpyridin-2-yl ester,

N-Methyl-N-phenylcarbamic acid 3-nitropyridin-2-yl ester, and

N-Methyl-N-phenylcarbamic acid 5-nitropyridin-2-yl ester

as well as diastereomers, enantiomers or tautomeric forms thereof including mixtures of these, pharmaceutically acceptable salts thereof, pharmaceutically acceptable solvates thereof, or polymorphs.

In one embodiment R² is selected from hydrogen, hydroxy, amino, halogen, C₁₋₆-alkyl, C₁₋₆-alkoxy, C₂₋₆-alkenyl, aryl, heteroaryl, C₃₋₈-heterocyclyl, amide, urea and thiourea and C₃₋₁₀-cycloalkyl, wherein each of hydroxy, amino, C₁₋₆-alkyl, C₁₋₆-alkoxy, C₂₋₆-alkenyl, aryl, heteroaryl, C₃₋₈-heterocyclyl, amide, urea and thiourea and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl.

In another embodiment R² is halogen or hydrogen.

In another embodiment R⁴ is selected from hydrogen, halogen, C₁₋₆-alkyl, C₂₋₆-alkenyl, wherein each of C₁₋₆-alkyl, C₂₋₆-alkenyl is optionally substituted with one or more substituents independently selected from hydroxy, halogen, C₁₋₆-alkyl, C₂₋₆-alkenyl, aryl, heteroaryl, C₃₋₈-heterocyclyl and C₃₋₁₀-cycloalkyl.

In another embodiment R⁴ is selected from hydrogen, halogen and C₁₋₆-alkyl.

In another embodiment R⁴ is hydrogen.

In another embodiment R⁴ is halogen.

In another embodiment R³ is selected from hydrogen, hydroxy, halogen, C₁₋₆-alkyl, C₂₋₆-alkenyl, wherein each of hydroxy, C₁₋₆-alkyl, C₂₋₆-alkenyl is optionally substituted with one or more substituents independently selected from hydroxy, halogen, C₁₋₆-alkyl, C₂₋₆-alkenyl, aryl, heteroaryl, C₃₋₈-heterocyclyl and C₃₋₁₀-cycloalkyl.

In another embodiment R³ is selected from halogen, C₁₋₆-alkyl, methoxy, perhalomethyl and perhalomethoxy.

In another embodiment R³ is selected from halogen, methyl, ethyl, isopropyl, methoxy and perhalomethyl.

In another embodiment R³ is selected from halogen, methyl, methoxy and perhalomethyl.

In another aspect the present invention relates to a compound of the general formula (I): 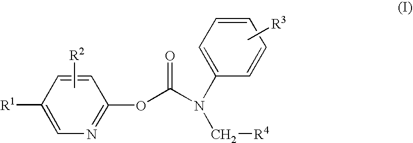
wherein
R¹ and R² are independently selected from hydrogen, hydroxy, sulfanyl, amino, amide, urea, thiourea, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, amide, urea, thiourea, sulfo, C₁-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R³, and R⁴ are independently selected from hydrogen, hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆alkyl, C₂₋₆-alkenyl, aryl, heteroaryl, C₃-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
with the proviso that said compound is not

2,2-Dimethyl-N-[6-(methyl-phenyl-carbamoyloxy)-pyridin-3-yl]-succinamic acid, 3,3-Dimethyl-4-[6-(methyl-phenyl-carbamoyloxy)-pyridin-3-ylcarbamoyl]-butyric acid, Methyl-phenyl-carbamic acid pyridin-2-yl ester, Methyl-phenyl-carbamic acid 5-chloro-pyridin-2-yl ester, Methyl-phenyl-carbamic acid 5-trifluoromethyl-pyridin-2-yl ester, Methyl-phenyl-carbamic acid 3-chloro-5-trifluoromethyl-pyridin-2-yl ester, Methyl-phenyl-carbamic acid 5-benzoylamino-pyridin-2-yl ester, Methyl-phenyl-carbamic acid 5-(cyclohexanecarbonyl-amino)-pyridin-2-yl ester, Methyl-phenyl-carbamic acid 4,4-dimethyl-2,6-dioxo-3,4,5,6-tetrahydro-2H-[1,3′]bipyridinyl-6′-yl ester, Methyl-phenyl-carbamic acid 5-(2,2-dimethyl-propionylamino)-pyridin-2-yl ester, Methyl-phenyl-carbamic acid 5-(2-cyclohexyl-acetylamino)-pyridin-2-yl ester, Methyl-phenyl-carbamic acid 5-(3,3-dimethyl-butyrylamino)-pyridin-2-yl ester, Methyl-phenyl-carbamic acid 5-[(pyridine-2-carbonyl)-amino]-pyridin-2-yl ester, Methyl-phenyl-carbamic acid 5-[(6-chloropyridine-3-carbonyl)-amino]-pyridin-2-yl ester, Methyl-phenyl-carbamic acid 5-(2,2-dimethyl-propylcarbamoyl)-pyridin-2-yl ester, Methyl-phenyl-carbamic acid 2,6-dioxo-3,4,5,6-tetrahydro-2H-[1,3′]bipyridinyl-6′-yl ester, Methyl-phenyl-carbamic acid 5-(2,5-dioxo-pyrrolidin-1-yl)-pyridin-2-yl ester, Methyl-phenyl-carbamic acid 5-(4-trifluoromethyl-benzoylamino)-pyridin-2-yl ester, Methyl-phenyl-carbamic acid 5-(4-chloro-benzoylamino)-pyridin-2-yl ester, Methyl-phenyl-carbamic acid 5-(4-methoxy-benzoylamino)-pyridin-2-yl ester, Methyl-phenyl-carbamic acid 4,4-dimethyl-3,4,5,6-tetrahydro-2H-[1,3′]bipyridinyl-6′-yl ester, Methyl-phenyl-carbamic acid 5-amino-pyridin-2-yl ester, Methyl-phenyl-carbamic acid 5-benzenesulfonylamino-pyridin-2-yl ester, Methyl-phenyl-carbamic acid 5-(3,3-dimethyl-2,5-dioxo-pyrrolidin-1-yl)-pyridin-2-yl ester, Methyl-phenyl-carbamic acid 5-[3,3-dimethyl-5-(4-methyl-piperazin-1-yl)-5-oxo-pentanoylamino]-pyridin-2-yl ester, Methyl-phenyl-carbamic acid 5-[3,3-dimethyl-4-(pyridin-3-ylcarbamoyl)-butyrylamino]-pyridin-2-yl ester, Methyl-phenyl-carbamic acid 5-(3,3-dimethyl-5-morpholin-4-yl-5-oxo-pentanoylamino)-pyridin-2-yl ester, Methyl-phenyl-carbamic acid 5-[4-(2-dimethylamino-ethylcarbamoyl)-3,3-dimethyl-butyrylamino]-pyridin-2-yl ester;

as well as diastereomers, enantiomers or tautomeric forms thereof including mixtures of these, pharmaceutically acceptable salts thereof, pharmaceutically acceptable solvates thereof, or polymorphs.

In one embodiment R² is selected from hydrogen, hydroxy, amino, halogen, C₁₋₆-alkyl, C₁₋₆-alkoxy, C₂₋₆-alkenyl, aryl, heteroaryl, C₃₋₈-heterocyclyl, amide, urea and thiourea and C₃₋₁₀-cycloalkyl, wherein each of hydroxy, amino, C₁₋₆-alkyl, C₁₋₆-alkoxy, C₂₋₆-alkenyl, aryl, heteroaryl, C₃₋₈-heterocyclyl, amide, urea and thiourea and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl.

In another embodiment R² is halogen or hydrogen.

In another embodiment R⁴ is selected from hydrogen, halogen, C₁₋₆-alkyl, C₂₋₆-alkenyl, wherein each of C₁₋₆alkyl, C₂₋₆-alkenyl is optionally substituted with one or more substituents independently selected from hydroxy, halogen, C₁₋₆-alkyl, C₂₋₆-alkenyl, aryl, heteroaryl, C₃₋₈-heterocyclyl and C₃₋₁₀-cycloalkyl.

In another embodiment R⁴ is selected from hydrogen, halogen and C₁₋₆-alkyl.

In another embodiment R⁴ is hydrogen.

In another embodiment R⁴ is halogen.

In another embodiment R³ is selected from hydrogen, halogen, C₁₋₆-alkyl, C₂₋₆-alkenyl, wherein each of, C₁₋₆-alkyl, C₂₋₆-alkenyl is optionally substituted with one or more substituents independently selected from hydroxy, halogen, C₁₋₆-alkyl, C₂₋₆-alkenyl, aryl, heteroaryl, C₃₋₈-heterocyclyl and C₃₋₁₀-cycloalkyl.

In another embodiment R³ is selected from halogen, C₁₋₆-alkyl, perhalomethyl and perhalomethoxy.

In yet another embodiment R³ is selected from halogen, methyl, ethyl, isopropyl and perhalomethyl.

In yet another embodiment R³ is selected from halogen, methyl or perhalomethyl.

In another aspect the present invention relates to a compound of the general formula (I): 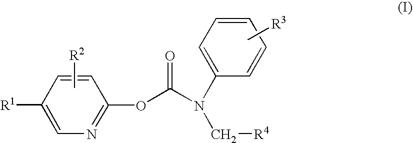
wherein
R¹ and R² are independently selected from hydrogen, hydroxy, sulfanyl, amino, halogen, sulfo, C₁₋₆alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl; R³, and R⁴ are independently selected from hydrogen, hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
with the proviso that said compound is not

2,2-Dimethyl-N-[6-(methyl-phenyl-carbamoyloxy)-pyridin-3-yl]-succinamic acid, 3,3-Dimethyl-4-[6-(methyl-phenyl-carbamoyloxy)-pyridin-3-ylcarbamoyl]-butyric acid, Methyl-phenyl-carbamic acid pyridin-2-yl ester, Methyl-phenyl-carbamic acid 5-chloro-pyridin-2-yl ester, Methyl-phenyl-carbamic acid 5-trifluoromethyl-pyridin-2-yl ester, Methyl-phenyl-carbamic acid 3-chloro-5-trifluoromethyl-pyridin-2-yl ester, Methyl-phenyl-carbamic acid 5-benzoylamino-pyridin-2-yl ester, Methyl-phenyl-carbamic acid 5-(cyclohexanecarbonyl-amino)-pyridin-2-yl ester, Methyl-phenyl-carbamic acid 4,4-dimethyl-2,6-dioxo-3,4,5,6-tetrahydro-2H-[1,3′]bipyridinyl-6′-yl ester, Methyl-phenyl-carbamic acid 5-(2,2-dimethyl-propionylamino)-pyridin-2-yl ester, Methyl-phenyl-carbamic acid 5-(2-cyclohexyl-acetylamino)-pyridin-2-yl ester, Methyl-phenyl-carbamic acid 5-(3,3-dimethyl-butyrylamino)-pyridin-2-yl ester, Methyl-phenyl-carbamic acid 5-[(pyridine-2-carbonyl)-amino]-pyridin-2-yl ester, Methyl-phenyl-carbamic acid 5-[(6-chloropyridine-3-carbonyl)-amino]-pyridin-2-yl ester, Methyl-phenyl-carbamic acid 5-(2,2-dimethyl-propylcarbamoyl)-pyridin-2-yl ester, Methyl-phenyl-carbamic acid 2,6-dioxo-3,4,5,6-tetrahydro-2H-[1,3′]bipyridinyl-6′-yl ester, Methyl-phenyl-carbamic acid 5-(2,5-dioxo-pyrrolidin-1-yl)-pyridin-2-yl ester, Methyl-phenyl-carbamic acid 5-(4-trifluoromethyl-benzoylamino)-pyridin-2-yl ester, Methyl-phenyl-carbamic acid 5-(4-chloro-benzoylamino)-pyridin-2-yl ester, Methyl-phenyl-carbamic acid 5-(4-methoxy-benzoylamino)-pyridin-2-yl ester, Methyl-phenyl-carbamic acid 4,4-dimethyl-3,4,5,6-tetrahydro-2H-[1,3′]bipyridinyl-6′-yl ester, Methyl-phenyl-carbamic acid 5-amino-pyridin-2-yl ester, Methyl-phenyl-carbamic acid 5-benzenesulfonylamino-pyridin-2-yl ester, Methyl-phenyl-carbamic acid 5-(3,3-dimethyl-2,5-dioxo-pyrrolidin-1-yl)-pyridin-2-yl ester, Methyl-phenyl-carbamic acid 5-[3,3-dimethyl-5-(4-methyl-piperazin-1-yl)-5-oxo-pentanoylamino]-pyridin-2-yl ester, Methyl-phenyl-carbamic acid 5-[3,3-dimethyl-4-(pyridin-3-ylcarbamoyl)-butyrylamino]-pyridin-2-yl ester, Methyl-phenyl-carbamic acid 5-(3,3-dimethyl-5-morpholin-4-yl-5-oxo-pentanoylamino)-pyridin-2-yl ester, Methyl-phenyl-carbamic acid 5-[4-(2-dimethylamino-ethylcarbamoyl)-3,3-dimethyl-butyrylamino]-pyridin-2-yl ester;

as well as diastereomers, enantiomers or tautomeric forms thereof including mixtures of these, pharmaceutically acceptable salts thereof, pharmaceutically acceptable solvates thereof, or polymorphs.

In another aspect the present invention is related to a compound of the general formula (I): 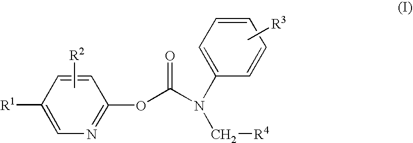
wherein
R² is H and R¹ is 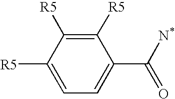
each R⁵ is independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R³, and R⁴ are independently selected from hydrogen, hydroxy, sulfanyl, halogen, amino, sulfo, C₁₋₆-alkyl, C₂₋₆-alkenyl, aryl, heteroaryl, Cam-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester,
as well as diastereomers, enantiomers or tautomeric forms thereof including mixtures of these, pharmaceutically acceptable salts thereof, pharmaceutically acceptable solvates thereof, or polymorphs.

In another aspect the present invention is related to a compound of the general formula (I): 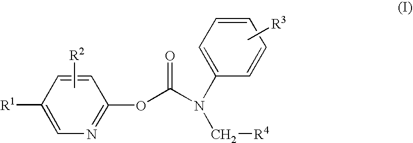
wherein
R² is H and R¹ is 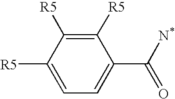
each R⁵ is independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R³, and R⁴ are independently selected from hydrogen, hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester,
as well as diastereomers, enantiomers or tautomeric forms thereof including mixtures of these, pharmaceutically acceptable salts thereof, pharmaceutically acceptable solvates thereof, or polymorphs.

In another aspect the present invention is related to a compound of the general formula (I): 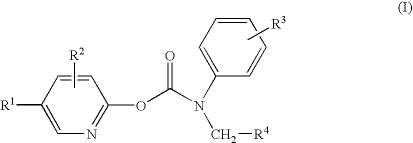
wherein
R² is H and R¹ is 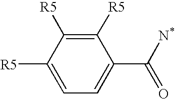
each R⁵ is independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R³, and R⁴ are hydrogen
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester,
as well as diastereomers, enantiomers or tautomeric forms thereof including mixtures of these, pharmaceutically acceptable salts thereof, pharmaceutically acceptable solvates thereof, or polymorphs.

In another aspect the present invention is related to a compound of the general formula (I): 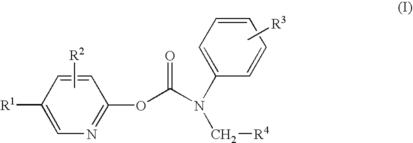
wherein
R² is H and R¹ is 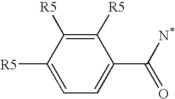
each R⁵ is independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl; R³, and R⁴ are hydrogen
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester,
as well as diastereomers, enantiomers or tautomeric forms thereof including mixtures of these, pharmaceutically acceptable salts thereof, pharmaceutically acceptable solvates thereof, or polymorphs.

In another aspect the present invention is related to a compound of the general formula (I): 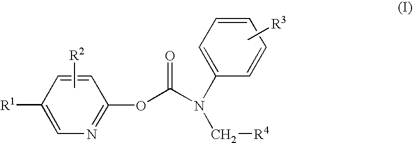
wherein
R² is H and R¹ is 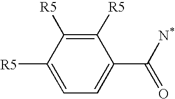
each R⁵ is independently selected from hydrogen, hydroxy, amino, halogen, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl,
R³ and R⁴ are hydrogen
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester,
as well as diastereomers, enantiomers or tautomeric forms thereof including mixtures of these, pharmaceutically acceptable salts thereof, pharmaceutically acceptable solvates thereof, or polymorphs.

In another aspect the present invention is related to a compound of the general formula (I): 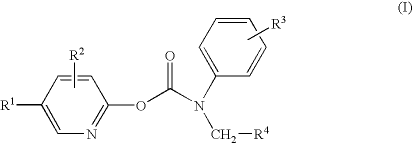
wherein
R² is H and R¹ is 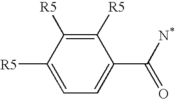
each R⁵ is independently selected from hydrogen, hydroxy, amino, halogen, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, amino, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl,
R³ and R⁴ are hydrogen PS with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester,
as well as diastereomers, enantiomers or tautomeric forms thereof including mixtures of these, pharmaceutically acceptable salts thereof, pharmaceutically acceptable solvates thereof, or polymorphs.

In one embodiment the invention is related to a compound wherein R² is selected from from hydrogen, hydroxy, amino, halogen, C₁₋₆-alkyl, C₁₋₆alkoxy, C₂₋₆-alkenyl, aryl, heteroaryl, C₃₋₈-heterocyclyl and C₃₋₁₀-cycloalkyl, wherein each of hydroxy, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl.

In one embodiment the invention is related to a compound wherein R³ is selected from fluor, chlor, methyl, perhalomethyl or perhalomethoxy.

In one embodiment the invention is related to a compound wherein R² is hydrogen.

In another embodiment the invention is related to a compound wherein R² is selected from the group consisting of

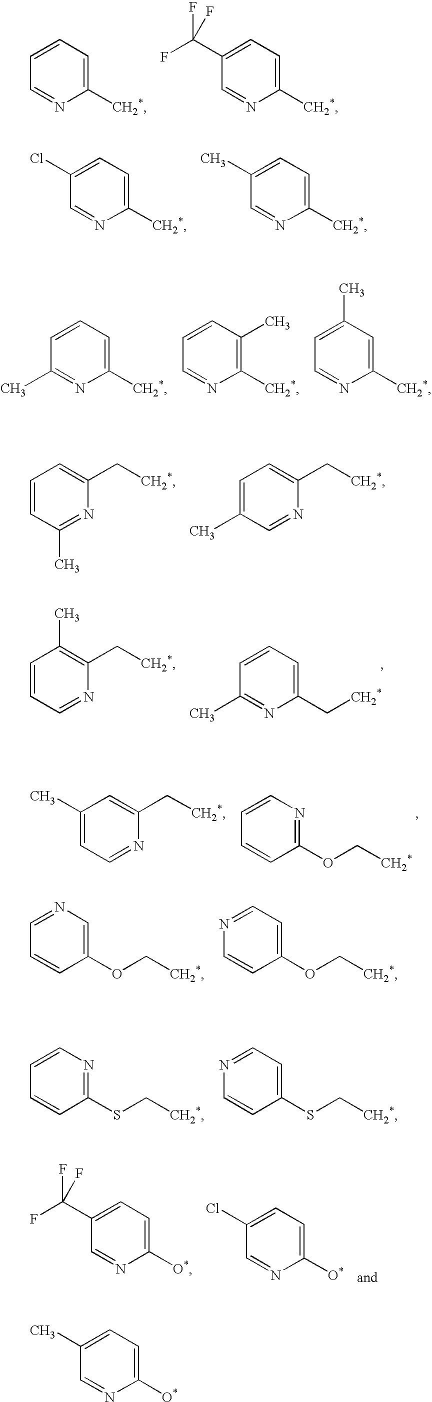

In another embodiment R² is selected from the group consisting of 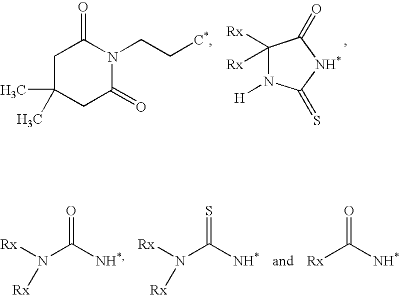
where each Rx is independently selected from hydroxy, sulfanyl, oxo, halogen, amino, sulfo, C₁₋₆-alkyl, C₁₋₆alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆alkyl, C₁₋₆-alkoxy, C₂₋₆-alkenyl, aryl, heteroaryl, C₃₋₈-heterocyclyl, and C₃₋₁₀-cycloalkyl;

In yet another embodiment R² is selected from the group consisting of 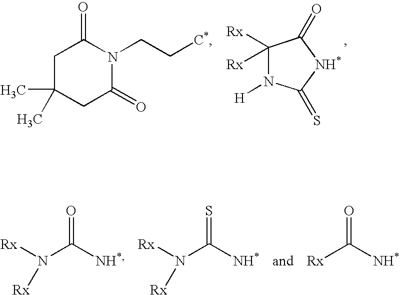
where each Rx is independently selected from hydroxy, sulfanyl, oxo, halogen, amino, sulfo, perhalomethyl, perhalomethoxy, C₁₋₆-alkyl, C₁₋₆alkoxy, C₂₋₆-alkenyl, aryl, heteroaryl, C₃₋₈-heterocyclyl, and C₃₋₁₀-cycloalkyl;

In another embodiment R² is selected from the group consisting of 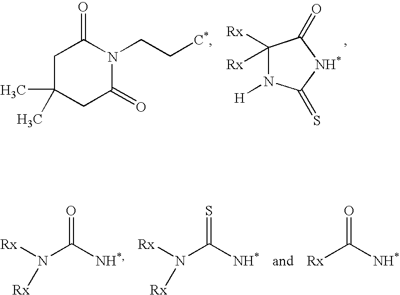
where each Rx is independently selected from hydroxy, sulfanyl, oxo, halogen, amino, sulfo, C₁₋₆-alkyl, C₁₋₆alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆alkyl, C₁₋₆alkoxy, C₂₋₆-alkenyl, aryl, heteroaryl, C₃₋₈-heterocyclyl, and C₃₋₁₀-cycloalkyl;

In another embodiment R² is selected from the group consisting of 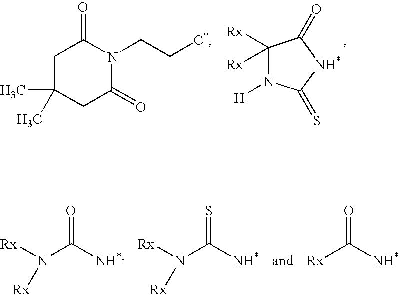
where each Rx is independently selected from hydroxy, sulfanyl, oxo, halogen, amino, sulfo, perhalomethyl, perhalomethoxy, C₁₋₆-alkyl, C₁₋₆alkoxy, C₂₋₆-alkenyl, aryl, heteroaryl, C₃₋₈-heterocyclyl, and C₃₋₁₀-cycloalkyl;
In another embodiment R² is selected from the group consisting of

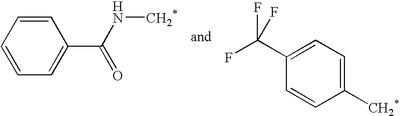

In one embodiment the invention is related to a compound wherein R² is selected from the group consisting of

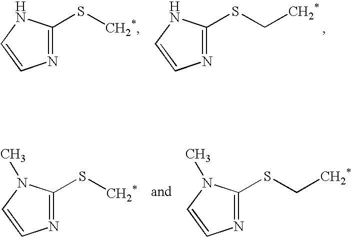

In one embodiment the invention is related to a compound wherein R² is selected from the group consisting of

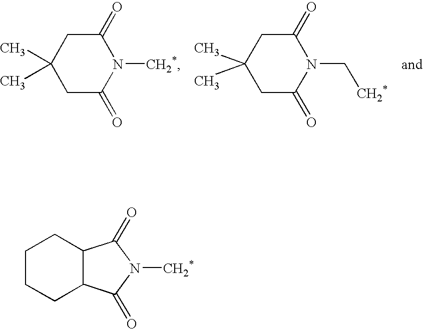

In one embodiment the invention is related to a compound wherein R¹ is selected from the group consisting of

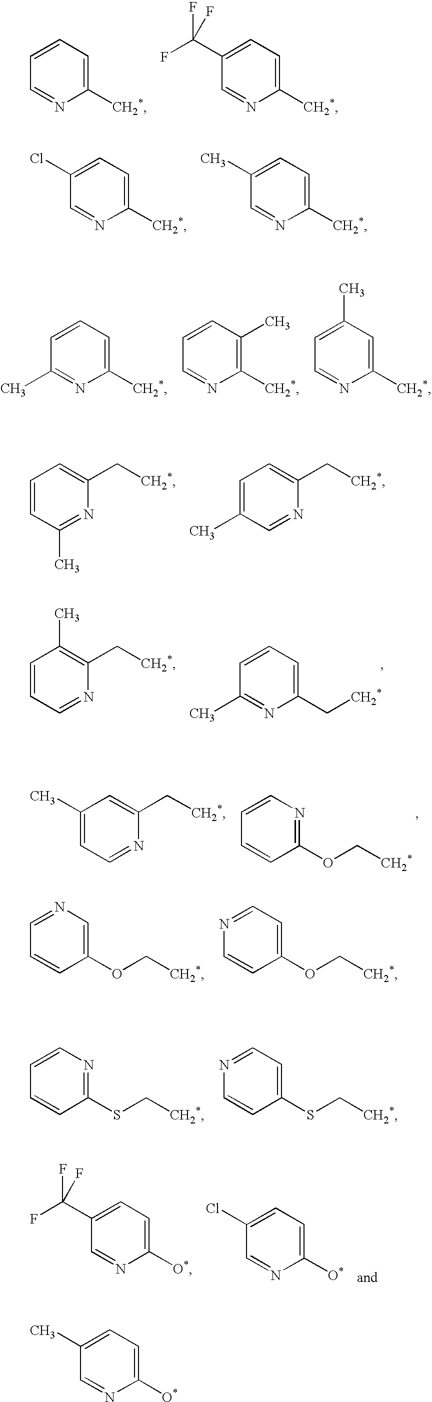

In one embodiment the invention is related to a compound wherein R¹ is selected from the group consisting of

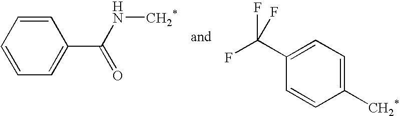

In one embodiment the invention is related to a compound wherein R¹ is selected from the group consisting of

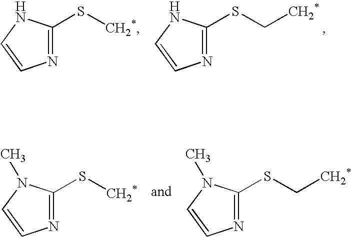

In another embodiment the invention is related to a compound wherein R¹ is selected from the group consisting of

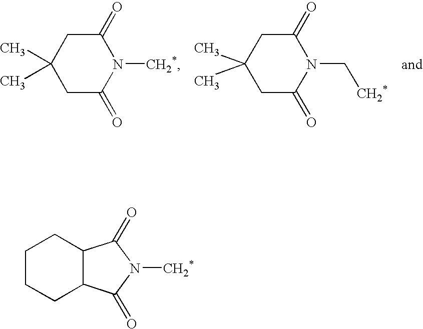

In another embodiment of the invention is related to a compound wherein R¹ is selected from the group consisting of 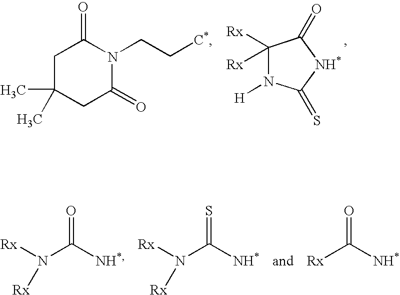
where each Rx is independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

In another embodiment of the invention is related to a compound wherein R¹ is selected from the group consisting of 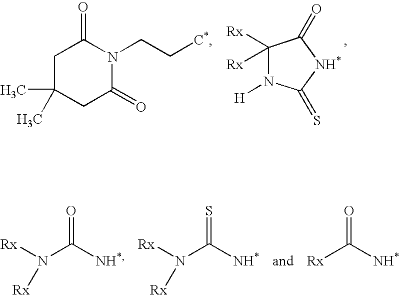
where each Rx is independently selected from hydroxy, sulfanyl, oxo, halogen, amino, sulfo, perhalomethyl, perhalomethoxy, C₁₋₆-alkyl, C₁₋₆alkoxy, C₂₋₆-alkenyl, aryl, heteroaryl, C₃₋₈-heterocyclyl, and C₃₋₁₀-cycloalkyl;

In another embodiment of the invention is related to a compound wherein R¹ is selected from the group consisting of 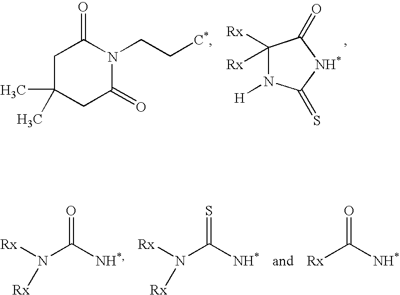
where each Rx is independently selected from halogen, perhalomethyl, perhalomethoxy, C₁₋₁-alkyl, heteroaryl, C₃₋₈-heterocyclyl, and C₃₋₁₀-cycloalkyl;

In another embodiment of the invention is related to a compound wherein R³ is hydrogen.

In another embodiment of the invention is related to a compound wherein R⁴ is hydrogen.

In another embodiment of the invention is related to a compound wherein R⁴ is selected from fluor, chlor, methyl, perhalomethyl or perhalomethoxy.

In another embodiment the invention is concerned with compounds, having one free —COOH group.

In another embodiment the invention is concerned with compounds having one free amino group, or one monosubstituted amino group or one disubstituted amino group.

In another embodiment the invention is concerned with compounds having one substituted or unsubstituted pyridine ring.

In another embodiment the invention is concerned with compounds having one substituted or unsubstituted imidazole ring.

In another embodiment the invention is concerned with compounds wherein the molar weight of said compound is less than 650 g/mole.

The property cLog P of a compound which has no ionisable group is calculated using Sybyl 6.6 from Tripos Corporation, version 4.0 (provided by Biobyte Corp., Claremont Calif., USA).

In another embodiment the invention is concerned with compounds wherein the compound contains no ionisable group and wherein cLog P is in the range from 1.0 to 6.0.

In another embodiment the invention is concerned with compounds wherein the compound contains no ionisable group and wherein cLog P is in the range from 1.0 to 5.0.

In another embodiment the invention is concerned with compounds wherein the compound contains no ionisable group and wherein cLog P is in the range from 1.0 to 4.0.

In another embodiment the invention is concerned with compounds wherein the compound contains no ionisable group and wherein cLog P is in the range from 2.0 to 4.0.

A number of other properties of the compounds are calculated using Sybyl 6.6. from Tripos Corporation, i.e. the number of H-bond donors, the number of H-bond acceptors, the number of rotatable bonds. The polar surface area (PSA) is calculated using the SAVol program Based on SAVol 3.7 using Allinger vdw radii. Polar atoms are oxygens, nitrogens, plus hydrogens attached to O and N developed by R. S. Pearlman, J. M. Skell and F. Deanda, Laboratory for Molecular Graphics and Theoretical Modeling, College of Pharmacy, University of Texas, Austin, Tex. 78712, U.S.A.

In another embodiment the invention is concerned with compounds wherein the ACD LogD is in the range from 0.8 to 3.0.

In another embodiment the invention is concerned with compounds wherein the number of H-bond donors is 0, 1, 2 or 3.

In another embodiment the invention is concerned with compounds wherein the number of H-bond donors is 0, 1, or 2.

In another embodiment the invention is concerned with compounds wherein the number of H-bond acceptors is in the range from 4 to 9.

In another embodiment the invention is concerned with compounds wherein the number of H-bond acceptors is in the range from 6 to 8.

In another embodiment the invention is concerned with compounds wherein the number of rotatable bonds of said compound is in the range from 4 to 14.

In another embodiment the invention is concerned with compounds wherein the number of rotatable bonds of said compound is in the range from 8 to 12.

In another embodiment the invention is concerned with compounds wherein the polar surface area (PSA) is in the range from 50 Å² to 120 Å².

In another embodiment the invention is concerned with compounds wherein the polar surface area (PSA) is in the range from 60 Å² to 100 Å².

In another embodiment the invention is concerned with a compound selected from the group consisting of
[Methyl-phenyl-carbamic acid 5-[2-(4,4-dimethyl-2,6-dioxo-piperidin-1-yl)-ethyl]-pyridin-2-yl ester], [[6-(Methyl-phenyl-carbamoyloxy)-pyridin-3-ylamino]-acetic acid tert-butyl ester],
[[6-(Methyl-phenyl-carbamoyloxy)-pyridin-3-ylamino]-acetic acid],
[Methyl-phenyl-carbamic acid 5-isothiocyanato-pyridin-2-yl ester],
[Methyl-phenyl-carbamic acid 5-(5-oxo-2-thioxo-imidazolidin-1-yl)-pyridin-2-yl ester],
[[6-(Methyl-phenyl-carbamoyloxy)-pyridin-3-ylamino]-acetic acid methyl ester],
[Methyl-phenyl-carbamic acid 5-(4,4-dimethyl-5-oxo-2-thioxo-imidazolidin-1-yl)-pyridin-2-yl ester],
[Methyl-phenyl-carbamic acid 5-(4-ethyl-5-oxo-2-thioxo-imidazolidin-1-yl)-pyridin-2-yl ester],
[(S)-Methyl-phenyl-carbamic acid 5-(4-isopropyl-5-oxo-2-thioxo-imidazolidin-1-yl)-pyridin-2-yl ester],
[Methyl-phenyl-carbamic acid 5-(4-tert-butoxymethyl-5-oxo-2-thioxo-imidazolidin-1-yl)-pyridin-2-yl ester],
[Methyl-phenyl-carbamic acid 5-(3-tert-butyl-thioureido)-pyridin-2-yl ester],
[Methyl-phenyl-carbamic acid 5-[3-(2,2-dimethyl-propyl)-thioureido]-pyridin-2-yl ester],
[Methyl-phenyl-carbamic acid 5-(3-isopropyl-thioureido)-pyridin-2-yl ester],
[Methyl-phenyl-carbamic acid 5-(3,3-diethyl-thioureido)-pyridin-2-yl ester],
[Methyl-phenyl-carbamic acid 5-(3-cyclohexyl-thioureido)-pyridin-2-yl ester],
[Methyl-phenyl-carbamic acid 5-(3-butyl-thioureido)-pyridin-2-yl ester],
[Methyl-phenyl-carbamic acid 5-(3-isobutyl-thioureido)-pyridin-2-yl ester],
[Methyl-phenyl-carbamic acid 5-(4-cyano-benzoylamino)-pyridin-2-yl ester],
[Methyl-phenyl-carbamic acid 5-(2-methyl-benzoylamino)-pyridin-2-yl ester],
[Methyl-phenyl-carbamic acid 5-(4-fluoro-benzoylamino)-pyridin-2-yl ester],
[Methyl-phenyl-carbamic acid 5-(3-methoxy-benzoylamino)-pyridin-2-yl ester],
[Methyl-phenyl-carbamic acid 5-(2-methoxy-benzoylamino)-pyridin-2-yl ester],
[Methyl-phenyl-carbamic acid 5-(3,4-dichloro-benzoylamino)-pyridin-2-yl ester],
[Methyl-phenyl-carbamic acid 5-(4-methyl-benzoylamino)-pyridin-2-yl ester],
[Methyl-phenyl-carbamic acid 5-(3-bromo-benzoylamino)-pyridin-2-yl ester],
[Methyl-phenyl-carbamic acid 5-(3-cyano-benzoylamino)-pyridin-2-yl ester],
[Methyl-phenyl-carbamic acid 5-(2-trifluoromethoxy-benzoylamino)-pyridin-2-yl ester],
[Methyl-phenyl-carbamic acid 5-(2-fluoro-3-trifluoromethyl-benzoylamino)-pyridin-2-yl ester],
[Methyl-phenyl-carbamic acid 5-(3-trifluoromethyl-benzoylamino)-pyridin-2-yl ester],
[Methyl-phenyl-carbamic acid 5-(3,4-difluoro-benzoylamino)-pyridin-2-yl ester],
[Methyl-phenyl-carbamic acid 5-(3-dimethylamino-benzoylamino)-pyridin-2-yl ester],
[Methyl-phenyl-carbamic acid 5-(3-dimethylamino-benzoylamino)-pyridin-2-yl ester],
[Methyl-phenyl-carbamic acid 5-(4-nitro-benzoylamino)-pyridin-2-yl ester],
[Methyl-phenyl-carbamic acid 5-(4-amino-benzoylamino)-pyridin-2-yl ester],
[Methyl-phenyl-carbamic acid 5-(3-tert-butyl-ureido)-pyridin-2-yl ester],
[Methyl-phenyl-carbamic acid 5-(4-formyl-benzoylamino)-pyridin-2-yl ester],
[Methyl-phenyl-carbamic acid 5-(4-morpholin-4-ylmethyl-benzoylamino)-pyridin-2-yl ester],
[Methyl-phenyl-carbamic acid 5-(4-hydroxymethyl-benzoylamino)-pyridin-2-yl ester],
[Methyl-phenyl-carbamic acid 5-(4-chloromethyl-benzoylamino)-pyridin-2-yl ester],
[Methyl-phenyl-carbamic acid 5-(4-piperidin-1-ylmethyl-benzoylamino)-pyridin-2-yl ester],
[Methyl-phenyl-carbamic acid 5-[4-(4-methyl-piperidin-1-ylmethyl)-benzoylamino]-pyridin-2-yl ester],
[Methyl-phenyl-carbamic acid 5-[4-(2-ethyl-piperidin-1-ylmethyl)-benzoylamino]-pyridin-2-yl ester],
[Methyl-phenyl-carbamic acid 5-[4-(2-methyl-piperidin-1-ylmethyl)-benzoylamino]-pyridin-2-yl ester],
[Methyl-phenyl-carbamic acid 5-[4-(3-methyl-piperidin-1-ylmethyl)-benzoylamino]-pyridin-2-yl ester],
Methyl-phenyl-carbamic acid 5-(4-piperidin-1-yl-benzoylamino)-pyridin-2-yl ester
Methyl-phenyl-carbamic acid 5-[4-(2-methyl-piperidin-1-yl)-benzoylamino]-pyridin-2-yl ester,
Methyl-phenyl-carbamic acid 5-[4-(3-methyl-piperidin-1-yl)-benzoylamino]-pyridin-2-yl ester
Methyl-phenyl-carbamic acid 5-[4-(4-methyl-piperidin-1-yl)-benzoylamino]-pyridin-2-yl ester-
Methyl-phenyl-carbamic acid 4-[4-(2-ethyl-piperidin-1-yl)-benzoylamino]-pyridin-2-yl ester-
Methyl-phenyl-carbamic acid 4-[4-(4,4-dimethyl-piperidin-1-yl)-benzoylamino]-pyridin-2-yl ester
Methyl-phenyl-carbamic acid 4-[4-(2,6-dimethyl-piperidin-1-yl)-benzoylamino]-pyridin-2-yl ester
Methyl-phenyl-carbamic acid 4-[4-(2,4,6-trimethyl-piperidin-1-yl)-benzoylamino]-pyridin-2-yl ester
Methyl-phenyl-carbamic acid 4-[4-(2,6-dimethyl-piperidin-1-ylmethyl)-benzoylamino]-pyridin-2-yl ester
Methyl-phenyl-carbamic acid 4-[4-(4,4-dimethyl-piperidin-1-ylmethyl)-benzoylamino]-pyridin-2-yl ester
Methyl-phenyl-carbamic acid 4-[4-(2,4,6-trimethyl-piperidin-1-ylmethyl)-benzoylamino]-pyridin-2-yl ester
Methyl-phenyl-carbamic acid 5-[4-(2-piperidin-1-yl-ethyl)-benzoylamino]-pyridin-2-yl ester
Methyl-phenyl-carbamic acid 5-{4-[2-(2-methyl-piperidin-1-yl)-ethyl]-benzoylamino}-pyridin-2-yl ester
Methyl-phenyl-carbamic acid 5-{4-[2-(3-methyl-piperidin-1-yl)-ethyl]-benzoylamino}-pyridin-2-yl ester
Methyl-phenyl-carbamic acid 5-{4-[2-(4-methyl-piperidin-1-yl)-ethyl]-benzoylamino}-pyridin-2-yl ester
Methyl-phenyl-carbamic acid 5-{4-[2-(2-ethyl-piperidin-1-yl)-ethyl]-benzoylamino}-pyridin-2-yl ester
Methyl-phenyl-carbamic acid 5-{4-[2-(4,4-dimethyl-piperidin-1-yl)-ethyl]-benzoylamino}-pyridin-2-yl ester
Methyl-phenyl-carbamic acid 5-{4-[2-(2,6-dimethyl-piperidin-1-yl)-ethyl]-benzoylamino}-pyridin-2-yl ester
Methyl-phenyl-carbamic acid 5-{4-[2-(2,4,6-trimethyl-piperidin-1-yl)-ethyl]-benzoylamino}-pyridin-2-yl ester

In another embodiment the present invention relates to a compound selected from the group consisting of
Methyl-phenyl-carbamic acid 5-(4-dimethylaminomethyl-benzoylamino)-pyridin-2-yl ester,
Methyl-phenyl-carbamic acid 5-(4-diethylaminomethyl-benzoylamino)-pyridin-2-yl ester,
Methyl-phenyl-carbamic acid 5-(4-pyrrolidin-1-ylmethyl-benzoylamino)-pyridin-2-yl ester,
Methyl-phenyl-carbamic acid 5-(4-dipropylaminomethyl-benzoylamino)-pyridin-2-yl ester,
Methyl-phenyl-carbamic acid 5-(4-pyrrolidin-1-ylmethyl-benzoylamino)-pyridin-2-yl ester,
cis-Methyl-phenyl-carbamic acid 5-[4-(2,6-dimethyl-piperidin-1-ylmethyl)-benzoylamino]-pyridin-2-yl ester,
Methyl-phenyl-carbamic acid 5-[4-(4-oxo-piperidin-1-ylmethyl)-benzoylamino]-pyridin-2-yl ester,
cis-Methyl-phenyl-carbamic acid 5-[4-(2,6-dimethyl-morpholin-4-ylmethyl)-benzoylamino]-pyridin-2-yl ester,
Methyl-phenyl-carbamic acid 5-(4-thiomorpholin-4-ylmethyl-benzoylamino)-pyridin-2-yl ester,
Methyl-phenyl-carbamic acid 5-[3-(2-hydroxy-1,1-dimethyl-ethyl)-thioureido]-pyridin-2-yl ester,
Methyl-phenyl-carbamic acid 5-[3-(1-methyl-cyclopropyl)-thioureido]-pyridin-2-yl ester,
Methyl-phenyl-carbamic acid 5-[3-(1-methyl-cyclobutyl)-thioureido]-pyridin-2-yl ester,
Methyl-phenyl-carbamic acid 5-(4-imidazol-1-yl-benzoylamino)-pyridin-2-yl ester,
Methyl-phenyl-carbamic acid 5-(4-diethylamino-benzoylamino)-pyridin-2-yl ester,
Methyl-phenyl-carbamic acid 5-(4-[1,2,4]triazol-1-yl-benzoylamino)-pyridin-2-yl ester,
Methyl-phenyl-carbamic acid 5-(3,3-dipropyl-thioureido)-pyridin-2-yl ester,
Methyl-phenyl-carbamic acid 5-(3,3-dibutyl-thioureido)-pyridin-2-yl ester,
Methyl-phenyl-carbamic acid 5-[(piperidine-1-carbothioyl)-amino]-pyridin-2-yl ester,
Methyl-phenyl-carbamic acid 5-[(4-methyl-piperidine-1-carbothioyl)-amino]-pyridin-2-yl ester,
Methyl-phenyl-carbamic acid 5-[(4,4-dimethyl-piperidine-1-carbothioyl)-amino]-pyridin-2-yl ester,
(4-Bromo-phenyl)-methyl-carbamic acid 4,4-dimethyl-2,6-dioxo-3,4,5,6-tetrahydro-2H-[
1,3′]bipyridinyl-6′-yl ester,
(4-Chloro-phenyl)-methyl-carbamic acid 4,4-dimethyl-2,6-dioxo-3,4,5,6-tetrahydro-2H-[
1,3′]bipyridinyl-6′-yl ester,
(3,4-Dichloro-phenyl)-methyl-carbamic acid 4,4-dimethyl-2,6-dioxo-3,4,5,6-tetrahydro-2H-[
1,3′]bipyridinyl-6′-yl ester,
(3-Chloro-phenyl)-methyl-carbamic acid 4,4-dimethyl-2,6-dioxo-3,4,5,6-tetrahydro-2H-[
1,3′]bipyridinyl-6′-yl ester,
Methyl-p-tolyl-carbamic acid 4,4-dimethyl-2,6-dioxo-3,4,5,6-tetrahydro-2H-[1,3′]bipyridinyl-6′-yl ester,
(3-Fluoro-phenyl)-methyl-carbamic acid 4,4-dimethyl-2,6-dioxo-3,4,5,6-tetrahydro-2H-[
1,3′]bipyridinyl-6′-yl ester,
Methyl-m-tolyl-carbamic acid 4,4-dimethyl-2,6-dioxo-3,4,5,6-tetrahydro-2H-[1,3′]bipyridinyl-6′-yl ester,
(4-Methoxy-phenyl)-methyl-carbamic acid 4,4-dimethyl-2,6-dioxo-3,4,5,6-tetrahydro-2H-[
1,3′]bipyridinyl-6′-yl ester,
(3-Methoxy-phenyl)-methyl-carbamic acid 4,4-dimethyl-2,6-dioxo-3,4,5,6-tetrahydro-2H-[
1,3′]bipyridinyl-6′-yl ester,
Methyl-(3-trifluoromethyl-phenyl)-carbamic acid 4,4-dimethyl-2,6-dioxo-3,4,5,6-tetrahydro-2H-[
1,3′]bipyridinyl-6′-yl ester,
(3-Bromo-phenyl)-methyl-carbamic acid 4,4-dimethyl-2,6-dioxo-3,4,5,6-tetrahydro-2H-[
1,3′]bipyridinyl-6′-yl ester,
(4-Fluoro-phenyl)-methyl-carbamic acid 4,4-dimethyl-2,6-dioxo-3,4,5,6-tetrahydro-2H-[
1,3′]bipyridinyl-6′-yl ester,
[4-(2-Hydroxy-ethyl)-phenyl]-methyl-carbamic acid 4,4-dimethyl-2,6-dioxo-3,4,5,6-tetrahydro-2H-[1,3′]bipyridinyl-6′-yl ester,

In another aspect the present invention relates to a pharmaceutical composition comprising a compound of general formula I or a pharmaceutically acceptable salt thereof together with a pharmaceutically acceptable carrier or diluent.

In one embodiment the invention is concerned with a pharmaceutical composition in unit dosage form, comprising from about 0.05 to about 2000 mg, preferably from about 0.1 to about 500 mg and even more preferable from about 1.0 to about 100 mg of said compound according to the invention or pharmaceutically acceptable salt thereof.

In another embodiment the invention is concerned with a pharmaceutical composition for use as a medicament for inhibiting the lipolytic activity of hormone-sensitive lipase against triacylglycerols, diacylglycerols, cholesterol acyl esters or steroid acyl esters, said composition comprising a compound according to the invention or a pharmaceutically acceptable salt thereof together with a pharmaceutically acceptable carrier or diluent.

In another embodiment the invention is concerned with a pharmaceutical composition which is for oral administration.

In another embodiment the invention is concerned with a pharmaceutical composition which is for nasal, transdermal, pulmonal, or parenteral administration.

In another aspect the present invention relates to use of a compound according to the inventtion for the preparation of a pharmaceutical composition.

In one embodiment the invention is concerned with use of a compound according to the invention for inhibition of hormone sensitive lipase.

In another embodiment the invention is concerned with use of a compound according to the invention for preparation of a pharmaceutical composition for inhibition of the lipolytic activity of hormone-sensitive lipase against triacylglycerols, diacylglycerols, cholesterol acyl esters or steroid acyl esters.

In another embodiment the invention is concerned with use of a compound according to the invention for the preparation of a pharmaceutical composition for the treatment or prevention of any disorder where it is desirable to modulate the plasma level of free fatty acids, glycerol, LDL-cholesterol, HDL-cholesterol, insulin and/or glucose; and/or modulate intracellular triacylglycerol and cholesterol ester stores, intracellular level of fatty acids, fatty acid esters such as diacylglycerols, phosphatidic acids, long chain acyl-CoA's as well as citrate or malonyl-CoA; and/or increase insulin sensitivity in adipose tissue, skeletal muscle, liver or pancreatic P cells; and/or modulate insulin secretion from pancreatic β cells.

In another embodiment the invention is concerned with the above use wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

In another embodiment the invention is concerned with the use of a compound according to the invention for the preparation of a pharmaceutical composition for the treatment and/or prevention of dyslipidemia.

In another embodiment the invention is concerned with the use of a compound according to the invention for the preparation of a pharmaceutical composition for the treatment and/or prevention of hyperlipidemia.

In another embodiment the invention is concerned with the use of a compound according to the invention for the preparation of a pharmaceutical composition for the treatment and/or prevention of hyperglycemia.

In another embodiment the invention is concerned with the use of a compound according to the invention for lowering HbA_{1C}.

In another embodiment the invention is concerned with the preparation of a pharmaceutical composition for the treatment and/or prevention of diabetes type 2.

In another embodiment the invention is concerned with the use of a compound according to the invention for the preparation of a pharmaceutical composition for the treatment and/or prevention impaired glucose tolerance.

In another embodiment the invention is concerned with the use of a compound according to the invention for the preparation of a pharmaceutical composition for the treatment and/or prevention of metabolic syndrome X.

In another embodiment the invention is concerned with the use of a compound according to the invention for the preparation of a pharmaceutical composition for the treatment and/or prevention of atheroschlerosis.

In another embodiment the invention is concerned with the use of a compound according to the invention for the preparation of a pharmaceutical composition for delaying or prevention of the progression from impaired glucose tolerance to diabetes type 2.

In another embodiment the invention is concerned with the use of a compound according to the invention for the preparation of a pharmaceutical composition for delaying or prevention of the progression from non-insulin requiring diabetes type 2 to insulin requiring diabetes type 2.

In another embodiment the invention is concerned with the use according to above indicationns wherein a further antidiabetic, antiobesity, antihypertensive or appetite regulating drug is used.

In another embodiment the invention is concerned with the use according to above indications, wherein metformin is also used.

In another aspect the present invention is related to a method of treating a disorder of a patient where modulation of the activity of hormone-sensitive lipase is desired, the method comprising administering to a subject in need thereof a therapeutically effective amount of a compound according to the present invention or a pharmaceutically acceptable salt thereof.

In one embodiment the invention is concerned with a method of treating a disorder of a patient where lowering of the activity of hormone-sensitive lipase is desired, the method comprising administering to a subject in need thereof a therapeutically effective amount of a compound according to the invention or a pharmaceutically acceptable salt thereof.

In another embodiment the invention is concerned with the above methods wherein said administration is carried out by the oral, nasal, transdermal, pulmonal, or parenteral route.

In another embodiment the invention is concerned with the above methods wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

In another embodiment the invention is concerned with the above methods wherein the therapeutically effective amount of the compound is from about 0.05 to about 2000 mg, preferably from about 0.1 to about 500 mg and even more preferable from about 1.0 to about 100 mg of said compound per day.

In another embodiment the invention is concerned with the above methods wherein a further antidiabetic, antiobesity, antihypertensive or appetite regulating drug is administered to the patient.

In another embodiment the invention is concerned with above methods wherein metformin is also administered to the patient.

In another aspect the present invention is related to a process P₁ for the preparation of a compound of the invention or its pharmaceutically acceptable salt, which comprises reacting the appropriate alcohol with the appropriate carbamoylating reagent in a solvent according to the reaction scheme P₁ 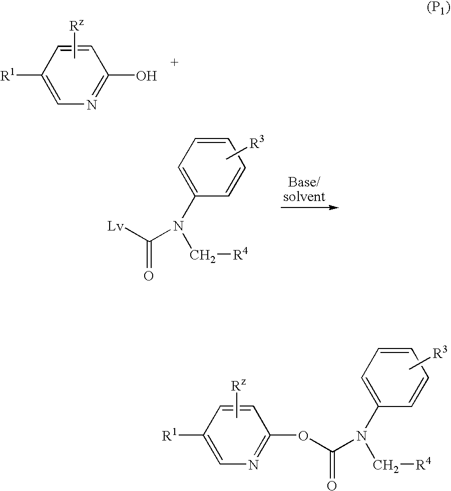
and isolating the disubstituted carbamate product.

In one embodiment of the invention wherein said carbamoylating reagent 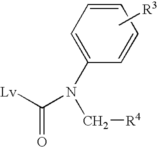
is selected from the group consisting of

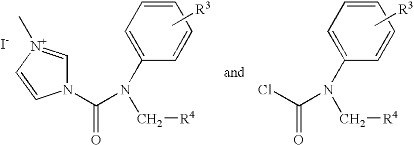

In another embodiment of the invention said solvent is selected from the group consisting of tetrahydrofurane, dimethylformamide and N-methylpyrolidone.

In another embodiment of the invention said base is selected from the group consisting of triethylamine, N,N-diisopropyl-N-ethylamine and DABCO.

In another aspect the present invention relates to a process P₂ for the preparation of a compound of the invention, said process comprising the treatment of the appropriate amine with the appropriate acylating reagent in a solvent and in the presence of a base according to the reaction scheme P₂ 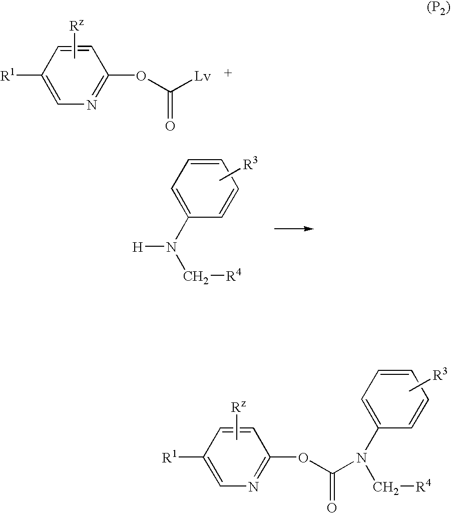
and isolating the disubstituted carbamate

In one embodiment of the invention Lv is Cl.

In another embodiment of the invention said solvent is selected from the group consisting of diethyl ether, tetrahydrofuran and dichloromethane.

In another embodiment of the invention said base is selected from the group consisting of trimethylamine, triethylamine, ethyl-diisopropyl-amine and 1,4-diazabicyclo[2.2.2]octane.

In another embodiment of the invention said base is present as a functionality in one or both of the substituents R³ and R⁴, thus forming a salt with the acid H-Lv.

The present invention also encompasses pharmaceutically acceptable salts of the present compounds. Such salts include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium, zinc, calcium salts and the like. Examples of amines and organic amines include ammonium, methylamine, dimethylamine, trimethylamine, ethylamine, diethylamine, propylamine, butylamine, tetramethylamine, ethanolamine, diethanolamine, triethanolamine, meglumine, ethylenediamine, choline, N,N′-dibenzylethylenediamine, N-benzylphenylethylamine, N-methyl-D-glucamine, guanidine and the like. Examples of cationic amino acids include lysine, arginine, histidine and the like.

The pharmaceutically acceptable salts are prepared by reacting the compound of formula I with 1 to 4 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol etc. Mixture of solvents may be used. Organic bases like lysine, arginine, diethanolamine, choline, guandine and their derivatives etc. may also be used. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid salicylic acid, hydroxynaphthoic acid, ascorbic acid, palmitic acid, succinic acid, benzoic acid, benzenesulfonic acid, tartaric acid and the like in solvents like ethyl acetate, ether, alcohols, acetone, THF, dioxane etc. Mixture of solvents may also be used.

The stereoisomers of the compounds forming part of this invention may be prepared by using reactants in their single enantiomeric form in the process wherever possible or by conducting the reaction in the presence of reagents or catalysts in their single enantiomer form or by resolving the mixture of stereoisomers by conventional methods. Some of the preferred methods include use of microbial resolution, enzymatic resolution, resolving the diastereomeric salts formed with chiral acids such as mandelic acid, camphorsulfonic acid, tartaric acid, lactic acid, and the like wherever applicable or chiral bases such as brucine, (R)- or (S)-phenylethylamine, cinchona alkaloids and their derivatives and the like. Commonly used methods are compiled by Jaques et al in “Enantiomers, Racemates and Resolution” (Wiley Interscience, 1981). More specifically the compound of formula I may be converted to a 1:1 mixture of diastereomeric amides by treating with chiral amines, aminoacids, aminoalcohols derived from aminoacids; conventional reaction conditions may be employed to convert acid into an amide; the dia-stereomers may be separated either by fractional crystallization or chromatography and the stereoisomers of compound of formula I may be prepared by hydrolysing the pure diastereomeric amide.

Various polymorphs of compound of general formula I forming part of this invention may be prepared by crystallization of compound of formula I under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the formula I. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The invention also relates to pharmaceutical compositions comprising, as an active ingredient, at least one compound of the formula I or any optical or geometric isomer or tautomeric form thereof including mixtures of these or a pharmaceutically acceptable salt thereof together with one or more pharmaceutically acceptable carriers or diluents.

Furthermore, the invention relates to the use of compounds of the general formula I or their tautomeric forms, their stereoisomers, their polymorphs, their pharmaceutically acceptable salts or pharmaceutically acceptable solvates thereof for the preparation of a pharmaceutical composition for the treatment and/or prevention of disorders where a decreased level of plasma FFA is desirable, such as the conditions mentioned above.

In another aspect, the present invention relates to a method of treating and/or preventing type 2 diabetes, insulin resistance, metabolic syndrome X, impaired glucose tolerance, dyslipidemia and abnormalities of lipoprotein metabolism.

In a still further aspect, the present invention relates to the use of one or more compounds of the general formula I, or pharmaceutically acceptable salts thereof, for the preparation of a pharmaceutical composition for the treatment and/or prevention of type 2 diabetes, insulin resistance, metabolic syndrome X, impaired glucose tolerance, dyslipidemia and abnormalities of lipoprotein metabolism.

In a still further aspect, the present compounds are useful for the delaying or prevention of the progression from impaired glucose tolerance to type 2 diabetes.

In a still further aspect, the present compounds are useful for the delaying or prevention of the progression from non-insulin requiring type 2 diabetes to insulin requiring type 2 diabetes.

In another aspect, the present compounds reduce triglyceride levels and are accordingly useful for the treatment and/or prevention of ailments and disorders such as diabetes and/or obesity.

In still another aspect, the compounds of general formula I are useful for the treatment of hyperglycemia, elevated HbA_{1c} level, hyperinsulinemia, type 1.5 diabetes, latent autoimmune diabetes in adults, maturity onset diabetes, beta-cell apoptosis, hemochromatosis induced diabetes, impaired glucose tolerance, impaired fasting glucose, metabolic syndrome X, insulin resistance, impaired lipid tolerance, cystic fibrosis related diabetes, polycystic ovarian syndrome, and gestational diabetes.

In still another aspect, the compounds of general formula I are useful for the treatment of obesity, dyslipidemia, diabetic dyslipidemia, hyperlipidemia, hypertriglyceridemia, hyperlipoproteinemia, hypercholesterolemia, hypertension, essential hypertension, acute hypertensive emergency, arteriosclerosis, atherosclerosis, restenosis, intermittent claudication (atherosclerosis oblitterens), cardiovascular disease, cardiomyopathy, cardiac hypertrophy, left ventricular hypertrophy, coronary artery disease, early coronary artery disease, heart insufficiency, exercise tolerance, chronic heart failure, mild chronic heart failure, arrhythmia, cardiac dysrythmia, syncopy, heart attack, myocardial infarction, Q-wave myocardial infarction, stroke, acute coronary syndrome, angina pectoris, unstable angina, cardiac bypass reocclusion, diastolic dysfunction, systolic dysfunction, non-Q-wave cardiac necrosis, catabolic changes after surgery, acute pancreatitis, and irritable bowel syndrome

In still another aspect, the compounds of general formula I may be useful for the treatment of diabetic retinopathy, background retinopathy, preproliferative retinopathy, proliferative retinopathy, macular edema, cataracts, nephropathy, nephrotic syndrome, diabetic nephropathy, microalbuminuria, macroalbuminuria, neuropathy, diabetic neuropathy, distal symmetrical sensorimotor polyneuropathy, and diabetic autonomic neuropathy.

In still another aspect, the compounds of general formula I are useful for increasing the number of beta—cells in a patient, increasing the size of beta—cells in a patient or stimulating beta—cell proliferation, modulating beta-cell function and insulin secretion in a patient in need thereof, which method comprises administration of an effective amount of a compound of formula I to a patient in need thereof.

The compounds of the invention are also believed to be useful for reducing body weight in a patient in need thereof.

The compounds of the invention are also believed to be useful for weight neutral treatment of above mentioned diseases.

The compounds of the invention are also believed to be useful for redistributing fat in a patient in need thereof.

The compounds of the invention are also believed to be useful for redistributing central fat in a patient in need thereof.

The compounds of the invention are also believed to be useful for reducing or preventing central obesity.

The compounds of the invention are also believed to be useful for reducing postprandial serum lipid excursions.

The compounds of the invention are also believed to be useful for the treatment of fatty acid oxidation disorders such as MCAD.

In still another aspect, the compounds of general formula I are believed to be useful for the treatment of a disease, condition or disorder wherein cholesterol is a precursor. Such diseases, conditions or disorders may relate to testosterone, e.g. male contraception, excessive testosterone levels, PCOS and prostate cancer. They may also relate to cortisol or corticotropin, e.g. Cushing disease.

The compounds of the invention are also believed to be useful for the treatment of cancer. Thus, the compounds of the general formula I may be useful for the treatment of insulinoma (pancreatic islet cell tumors), e.g. malignant insulinomas and multiple insulinomas, adipose cell carcinomas, e.g. lipocarconoma.

The compounds of the invention are also believed to be useful for the treatment of phaechromocytoma and other diseases with increased catecholamine incretion.

The compounds of the invention are also believed to be useful for the treatment of prostate cancer, e.g. adenocarcinoma.

In still another aspect, the compounds of general formula I may be useful for the treatment of hepatic steatosis.

In still another aspect, the compounds of general formula I may be useful for the treatment of cirrhosis.

In still another aspect, the compounds of general formula I may be useful for the treatment of AIDS or an AIDS related diseases, condition or disorders

In still another aspect, the compounds of general formula I may be useful for the treatment of lipodystrophy

In still another aspect, the compounds of general formula I may be useful for the treatment of lactic acidosis.

In yet another aspect, the compounds of the present invention are expected to be useful for the treatment of CNS diseases, conditions or disorders.

Thus, the compound of the present invention may be used for the treatment of Parkinsons disease, Alzheimers disease, ADHD (Attention Deficit Hyperactivity Disorder), feeding disorders such as bulimia and anorexia, depression, anxiety, cognitive memory disorders, age related cognitive decline, mild cognitive impairment and schizophrenia.

In yet another aspect, the compounds of the present invention may be useful for the treatment of inflammatory disorders, e.g. rheumatoid arthritis, psoriasis, systemic inflammatory response syndrome, sepsis and the like.

The present compounds may also be administered in combination with one or more further pharmacologically active substances eg., selected from antiobesity agents, antidiabetics, anti-hypertensive agents, agents for the treatment and/or prevention of complications resulting from or associated with diabetes and agents for the treatment and/or prevention of complications and disorders resulting from or associated with obesity.

Thus, in a further aspect of the invention the present compounds may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators or TR β agonists.

In one embodiment of the invention the antiobesity agent is leptin.

In another embodiment the antiobesity agent is dexamphetamine or amphetamine.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

Suitable antidiabetics comprise insulin, exendin-4, GLP-1 (glucagon like peptide-1) and derivatives thereof such as those disclosed in WO 98/08871 to Novo Nordisk A/S, which is incorporated herein by reference as well as orally active hypoglycaemic agents.

The orally active hypoglycaemic agents preferably comprise sulphonylureas, biguanides, meglitinides, glucosidase inhibitors, glucagon antagonists such as those disclosed in WO 99/01423 to Novo Nordisk A/S and Agouron Pharmaceuticals, Inc., GLP-1 agonists, potassium channel openers such as those disclosed in WO 97/26265 and WO 99/03861 to Novo Nordisk A/S which are incorporated herein by reference, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents as HMG CoA inhibitors (statins), compounds lowering food intake, RXR agonists and agents acting on the ATP-dependent potassium channel of the β-cells.

In one embodiment of the invention the present compounds are administered in combination with insulin.

In a further embodiment the present compounds are administered in combination with a sulphonylurea eg. tolbutamide, glibenclamide, glipizide or glicazide.

In another embodiment the present compounds are administered in combination with a biguanide eg. metformin.

In yet another embodiment the present compounds are administered in combination with a meglitinide eg. repaglinide or senaglinide.

In a further embodiment the present compounds are administered in combination with an α-glucosidase inhibitor eg. miglitol or acarbose.

In another embodiment the present compounds are administered in combination with an agent acting on the ATP-dependent potassium channel of the β-cells eg. tolbutamide, glibenclamide, glipizide, glicazide or repaglinide.

Furthermore, the present compounds may be administered in combination with nateglinide.

In still another embodiment the present compounds are administered in combination with an antihyperlipidemic agent or antilipidemic agent eg. cholestyramine, colestipol, clofibrate, gemfibrozil, lovastatin, pravastatin, simvastatin, probucol or dextrothyroxine.

In a further embodiment the present compounds are administered in combination with more than one of the above-mentioned compounds eg. in combination with a sulphonylurea and metformin, a sulphonylurea and acarbose, repaglinide and metformin, insulin and a sulphonylurea, insulin and metformin, insulin, insulin and lovastatin, etc.

Furthermore, the present compounds may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, alatriopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compounds according to the invention with one or more of the above-mentioned compounds and optionally one or more further pharmacologically active substances are considered to be within the scope of the present invention.

The present invention also relates to processes according to reaction schemes P₁ and P₂ for the preparation of the above said novel compounds, their derivatives, their analogs, their tautomeric forms, their stereoisomers, their polymorphs, their pharmaceutically acceptable salts or pharmaceutically acceptable solvates.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995. The compositions may appear in conventional forms, for example capsules, tablets, aerosols, solutions, suspensions or topical applications.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well-known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

The therapeutic dose of the compound will depend upon the frequency and mode of administration, the sex, age, weight and general condition of the subject treated, the nature and severity of the condition treated and any concomitant diseases to be treated and other factors evident to those skilled in the art. The formulations may conveniently be presented in unit dosage form by methods known to those skilled in the art. In one embodiment the composition in unit dosage form, comprises from about 0.05 to about 2000 mg, preferably from about 0.1 to about 500 mg of the compound of formula I pharmaceutically acceptable salt thereof.

In a still further embodiment the pharmaceutical composition is for oral, nasal, transdermal, pulmonal, or parenteral administration.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the invention contains a free base such salts are prepared in a conventional manner by treating a solution or suspension of a free base of the compound with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the present compounds in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of suitable carriers are water, salt solutions, alcohols, polyethylene glycols, polyhydroxyethoxylated castor oil, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents.

The pharmaceutical compositions formed by combining the compounds of the invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet which may be prepared by conventional tabletting techniques may contain:

| | |
|---|---|
| Core: | |
| Active compound (as free compound or salt thereof) | 5 mg |
| Colloidal silicon dioxide (Aerosil) | 1.5 mg |
| Cellulose, microcryst. (Avicel) | 70 mg |
| Modified cellulose gum (Ac-Di-Sol) | 7.5 mg |
| Magnesium stearate | q.s. |
| Coating: | |
| HPMC approx. | 9 mg |
| *Mywacett 9-40 T approx. | 0.9 mg |

*Acylated monoglyceride used as plasticizer for film coating.

The compounds of the invention may be administered to a patient which is a mammal, especially a human in need thereof. Such mammals include also animals, both domestic animals, e.g. household pets, and non-domestic animals such as wildlife.

In a further aspect of the invention the present compounds may be administered in combination with further pharmacologically active substances e.g. an antidiabetic or other pharmacologically active material, including other compounds for the treatment and/or prevention of insulin resistance and diseases, wherein insulin resistance is the pathophysiological mechanism.

Furthermore, the compounds according to the invention may be administered in combination with antiobesity agents or appetite regulating agents.

### EXAMPLES

Starting Materials

For the synthesis of methyl-phenyl-carbamic acid 5-amino-pyridin-2-yl ester, N-(6-hydroxy-pyridin-3-yl)-benzamide and 6′-hydroxy-4,4-dimethyl-4,5-dihydro-3H-[1,3′]bipyridinyl-2,6-dione see; PCT/DK02/00852. For the synthesis of (6-methoxy-pyridin-3-yl)-methanol see e.g.; Alan P. Kozikowski, Yan Xia, E. Rajarathnam Reddy, Werner Tuckmantel, Israel Hanin, and X. C. Tang

J. Org. Chem. 1991, 56, 4636-4645.

### Example 1

### [Methyl-phenyl-carbamic acid 5-[2-(4,4-dimethyl-2,6-dioxo-piperidin-1-yl)-ethyl]-pyridin-2-yl ester]

Step A:

Thionyl chloride (100 mL) was added to a stirred solution of (6-methoxy-pyridin-3-yl)-methanol (31.0 g, 223 mmol) in chloroform (300 mL). After refluxing for 2 hours, the solvent and excess thionyl chloride were removed by evaporation in vacuo. The residue was stripped with toluene, dissolved in dichloromethane (400 mL) and extracted with a saturated aqueous sodium bicarbonate solution (200 mL). The organic layer was dried over sodium sulphate, filtered and evaporated in vacuo, yielding a yellow oil. The oil was dissolved in dimethylformamide (750 mL), followed by the addition of potassium cyanide (14.5 g, 223 mmol). After stirring overnight at room temperature, the reaction mixture was poured into water (1.5 L). Ethyl acetate (500 mL) was added and both layers were separated. The water layer was extracted with ethyl acetate (4×200 mL) and the combined organic layers were dried over sodium sulphate, filtered and evaporated in vacuo. The product was purified by flash column chromatography (SiO₂, ethyl acetate/heptane 1/2) yielding (6-methoxy-pyridin-3-yl)-acetonitrile (13.7 g, 42% yield).

¹H NMR (200 MHz, CDCl₃): δ=3.75 (s, 2H), 4.00 (s, 3H), 6.85 (d, 1H), 7.60 (dd, 1H), 8.15 (br.s, 1H);

Step B:

Borane-tetrahydrofuran complex (1 M solution in tetrahydrofuran, 350 mL) was added to a stirred solution of (6-methoxy-pyridin-3-yl)-acetonitrile (13.7 g, 92.5 mmol) in tetrahydrofuran (130 mL). After stirring overnight at 60° C., 1N aqueous hydrochloric acid (80 mL) was added. The resulting mixture was heated at reflux for 2 hours followed by cooling to room temperature. The solution was washed with tert-butyl methyl ether. 9N aqueous sodium hydroxide (60 mL) was added to the water layer followed by extraction with dichloromethane (4×200 mL). The combined organic layers were dried over sodium sulphate/potassium carbonate 1/1, filtered and evaporated in vacuo, yielding an oil, which was redissolved in dichloromethane (100 mL). A 4 M solution of hydrochloric acid in dioxane was added. The white precipitate was isolated by filtration and dried, yielding 2-(6-methoxy-pyridin-3-yl)-ethylamine dihydrochloride (10.2 g, 49% yield).

¹H NMR (200 MHz, CDCl₃): δ=2.80-2.96 (m, 2H), 2.98-3.14 (m, 2H), 6.86 (d, 1H), 7.27 (dd, 1H), 8.12 (d, 1H), 8.20 (br.s, 2H).

Step C:

A solution of 2-(6-methoxy-pyridin-3-yl)-ethylamine dihydrochloride (0.94 g, 4.18 mmol), 3,3-dimethylglutaric anhydride (0.71 g, 4.99 mmol) and triethylamine (1.16 mL, 8.35 mmol) in dichloromethane was stirred at room temperature for 30 minutes. 1,1′-Carbonyldiimidazole (0.68 g, 4.18 mmol) was added and the solution was refluxed for 2 hours followed by stirring overnight at room temperature. An additional small amount of 1,1′-carbonyldiimidazole was added and reflux was continued for 3 hours. The solution was extracted twice with water, dried over sodium sulphate, filtered and evaporated in vacuo, yielding 1-[2-(6-methoxy-pyridin-3-yl)-ethyl]-4,4-dimethyl-piperidine-2,6-dione (1.22 g, 106% yield) as a colourless oil that solidified upon standing.

¹H NMR (300 MHz, CDCl₃): δ=1.04 (s, 6H), 2.48 (s, 4H), 2.74 (m, 2H), 3.91 (s, 3H), 3.96 (m, 2H), 6.68 (d, 1H), 7.50 (dd, 1H), 7.98 (d, 1H); HPLC-MS (Method A): m/z=277 (M+H)⁺; Rt=2.72.

Step D:

During 5 minutes HCl-gas was bubbled into a solution of 1-[2-(6-methoxy-pyridin-3-yl)-ethyl]-4,4-dimethyl-piperidine-2,6-dione (1.15 g, 4.18 mmol) in tetrahydrofuran (25 mL). Ether (200 mL) was added and the solvent was evaporated in vacuo. The residue was heated for 5 minutes at 180° C. After cooling to room temperature the solid was dissolved in dichloromethane (10 mL). 1,4-Diazabicyclo[2.2.2]octane (0.47 g, 4.18 mmol) and N-methyl-N-phenyl-carbamoyl chloride (0.71 g, 4.18 mmol) were added. After stirring for 30 minutes at room temperature, the product was purified by flash column chromatography (SiO₂, ethyl acetate/heptane 60/40) yielding the title compound (0.42 g, 25% yield) as a white solid.

¹H NMR (300 MHz, CDCl₃): δ=1.04 (s, 6H), 2.48 (s, 4H), 2.82 (m, 2H), 3.43 (br.s, 3H), 3.99 (m, 2H), 7.00 (br.s, 1H), 7.26 (m, 1H), 7.39 (d, 4H), 7.68 (dd, 1H), 8.20 (d, 1H); HPLC-MS (Method A): m/z=396 (M+H)⁺; Rt=3.60 min.

### Example 2

### [[6-(Methyl-phenyl-carbamoyloxy)-pyridin-3-ylamino]-acetic acid tert-butyl ester]

A mixture of methyl-phenyl-carbamic acid 5-amino-pyridin-2-yl ester (2.43 g, 10.0 mmol), bromoacetic acid tert-butylester (3.90 g, 20.0 mmol), potassium carbonate (5.52 g, 40.0 mmol) and a catalytic amount of 18-crown-6 in toluene (50 mL) was stirred for 4 days at room temperature. The solvent was evaporated in vacuo and the residue was purified by flash column chromatography (SiO₂, ethyl acetate/heptane 50/50) yielding the title compound (0.67 g, 19% yield) as an oil.

¹H NMR (300 MHz, CDCl₃): &=1.50 (s, 9H), 3.42 (br.s, 3H), 3.79 (s, 2H), 4.30 (br.s, 1H), 6.83-7.00 (m, 2H), 7.39 (m, 4H), 7.70 (d, 1H); HPLC-MS (Method A): m/z=358 (M+H)⁺; Rt=3.74 min.

### Example 3

### [[6-(Methyl-phenyl-carbamoyloxy)-pyridin-3-ylamino]-acetic acid]

A solution of [6-(methyl-phenyl-carbamoyloxy)-pyridin-3-ylamino]-acetic acid tert-butyl ester (0.60 g, 1.68 mmol) in 20% trifluoroacetic acid in dichloromethane (25 mL) was stirred at room temperature for 3 hours. The solvent was evaporated in vacuo yielding a solid, which was heated briefly with a small amount of ethyl acetate. After cooling to room temperature the solid was isolated by suction, yielding the title compound (0.42 g, 82% yield) as a white solid.

¹H NMR (300 MHz, DMSO-d₆): δ=3.33 (s, 3H), 3.89 (s, 2H), 6.96 (d, 1H), 7.14 (dd, 1H), 7.27 (m, 1H), 7.43 (m, 4H), 7.66 (d, 1H), 11.60 (br.s, 1H); HPLC-MS (Method A): m/z=302 (M+H)⁺; Rt=2.50 min.

### Example 4

### [Methyl-phenyl-carbamic acid 5-isothiocyanato-pyridin-2-yl ester]

A solution of methyl-phenyl-carbamic acid 5-amino-pyridin-2-yl ester (2.43 g, 10.0 mmol) and di-2-pyridyl thionocarbonate (2.32 g, 10.0 mmol) in dichloropropane (25 mL) was stirred at room temperature for 2 hours. The solution was used as such, without isolation of the product. HPLC-MS (Method A): m/z=308 (M+Na)⁺; Rt=4.08 min.

### Example 5

### [Methyl-phenyl-carbamic acid 5-(5-oxo-2-thioxo-imidazolidin-1-yl)-pyridin-2-yl ester]

Glycine hydrochloride (2.00 mmol, 251 mg) was added to a solution of methyl-phenyl-carbamic acid 5-isothiocyanato-pyridin-2-yl ester (0.57 g, 2.00 mmol) in 1,2-dichloropropane (5 mL). A few millilitres of methanol were added to get a clear solution. Triethylamine (0.28 mL, 2.00 mmol) was added and the solution was stirred overnight at room temperature. Dichloromethane was added and the reaction mixture was extracted twice with water, dried over sodium sulphate, filtered and evaporated in vacuo. The residue was purified by flash column chromatography (SiO₂, ethyl acetate/heptane 70/30), yielding the title compound (0.27 g, 40% yield) as a solid.

¹H NMR (300 MHz, CDCl₃): δ=3.46 (br.s, 3H), 4.14 (s, 2H), 7.17 (m, 1H), 7.27 (m, 1H), 7.39 (m, 4H), 7.75 (m, 2H), 8.39 (s, 1H); HPLC-MS (Method A): m/z=375 (M+H)⁺; Rt=2.64 min.

### Example 6

### [[6-(Methyl-phenyl-carbamoyloxy)-pyridin-3-ylamino]-acetic acid methyl ester]

A mixture of methyl-phenyl-carbamic acid 5-amino-pyridin-2-yl ester (2.43 g, 10.0 mmol), bromoacetic acid methylester (3.06 g, 20.0 mmol), potassium carbonate (5.52 g, 40.0 mmol) and a catalytic amount of 18-crown-6 in toluene (50 mL) was stirred for 3 days at room temperature. The solution was extracted twice with water, dried over sodium sulphate, filtered and evaporated in vacuo. The residue was crystallised from ethyl acetate/heptane, yielding the title compound (0.62 g, 20% yield) as a white solid.

¹H NMR (300 MHz, CDCl₃): δ=3.42 (br.s, 3H), 3.77 (s, 3H), 3.89 (s, 2H), 4.38 (br.s, 1H), 6.89 (m, 1H), 6.96 (dd, 1H), 7.24 (m, 1H), 7.37 (m, 4H), 7.70 (d, 1H); HPLC-MS (Method A): m/z=316 (M+H)⁺; Rt=2.87 min.

### Example 7

### [Methyl-phenyl-carbamic acid 5-(4,4-dimethyl-5-oxo-2-thioxo-imidazolidin-1-yl)-pyridin-2-yl ester]

Methyl-phenyl-carbamic acid 5-amino-pyridin-2-yl ester (1.22 g, 5.00 mmol) was added in portions to a stirred solution of di-2-pyridyl thionocarbonate (1.16 g, 5.00 mmol) in 1,2-dichloropropane (15 mL). After stirring for 1 hour at room temperature, alpha-aminoisobutyric acid methyl ester hydrochloride (0.77 g, 5.00 mmol) and triethylamine (0.7 mL) were added, together with some dimethylformamide to get a clear solution. After standing overnight the solvent was evaporated in vacuo and the residue was purified by flash column chromatography (SiO₂, ethyl acetate/heptane 50/50), yielding the title compound (1.30 g, 70% yield) as a white solid.

¹H NMR (300 MHz, CDCl₃): δ=1.48 (s, 6H), 3.45 (br.s, 3H), 7.19 (br.s, 1H), 7.28 (m, 1H), 7.39 (m, 4H), 7.79 (br.d, 1H), 8.35 (s, 1H, NH), 8.39 (s, 1H); HPLC-MS (Method A): m/z=371 (M+H)⁺; Rt=3.37 min.

### Example 8

### [Methyl-phenyl-carbamic acid 5-(4-ethyl-5-oxo-2-thioxo-imidazolidin-1-yl)-pyridin-2-yl ester]

DL-alpha-amino-n-butyric acid methyl ester hydrochloride (2.00 mmol, 307 mg) was added to a solution of methyl-phenyl-carbamic acid 5-isothiocyanato-pyridin-2-yl ester (0.57 g, 2.00 mmol) in 1,2-dichloropropane (5 mL). A small amount of methanol was added to get a clear solution. Triethylamine (0.28 mL, 2.00 mmol) was added and the solution was stirred overnight at room temperature. Dichloromethane was added and the reaction mixture was extracted twice with water, dried over sodium sulphate, filtered and evaporated in vacuo. The residue was purified by flash column chromatography (SiO₂, ethyl acetate/heptane 50/50), yielding the title compound (0.25 g, 33% yield) as a white solid.

¹H NMR (300 MHz, CDCl₃): δ=1.00 (t, 3H), 1.90 (m, 2H), 3.43 (br.s, 3H), 4.17 (t, 1H), 7.17 (br.s, 1H), 7.28 (m, 1H), 7.39 (m, 4H), 7.74 (br.d, 1H), 8.30 (s, 1H), 8.36 (s, 1H); HPLC-MS (Method A): m/z=371 (M+H)⁺; Rt=3.39 min.

### Example 9

### [(S)-Methyl-phenyl-carbamic acid 5-(4-isopropyl-5-oxo-2-thioxo-imidazolidin-1-yl)-pyridin-2-yl ester]

L-Valine methyl ester hydrochloride (2.00 mmol, 307 mg) was added to a solution of methyl-phenyl-carbamic acid 5-isothiocyanato-pyridin-2-yl ester (0.57 g, 2.00 mmol) in 1,2-dichloropropane (5 mL). A small amount of methanol was added to get a clear solution. Triethylamine (0.28 mL, 2.00 mmol) was added and the solution was stirred overnight at room temperature. Dichloromethane was added and the reaction mixture was extracted twice with water, dried over sodium sulphate, filtered and evaporated in vacuo. The residue was purified by flash column chromatography (SiO₂, ethyl acetate/heptane 50/50), yielding the title compound (139 mg, 18% yield) as a white solid.

¹H NMR (300 MHz, CDCl₃): δ=0.98 (d, 3H), 1.07 (d, 3H), 2.30 (m, 1H), 3.43 (br.s, 3H), 4.10 (d, 1H), 7.18 (br.s, 1H), 7.27 (m, 1H), 7.39 (m, 4H), 7.72 (br.d, 1H), 8.32 (s, 1H), 8.50 (s, 1H); HPLC-MS (Method A): m/z=385 (M+H)⁺; Rt=3.62 min.

### Example 10

### [Methyl-phenyl-carbamic acid 5-(4-tert-butoxymethyl-5-oxo-2-thioxo-imidazolidin-1-yl)-pyridin-2-yl ester]

O-t-Butyl-D-serine acid methyl ester hydrochloride (2.00 mmol, 307 mg) was added to a solution of methyl-phenyl-carbamic acid 5-isothiocyanato-pyridin-2-yl ester (0.57 g, 2.00 mmol) in dichloromethane (10 mL). Triethylamine (0.28 mL, 2.00 mmol) was added and the solution was stirred for 7 days at room temperature. Part of the solvent was removed by evaporation in vacuo. The residue was purified by flash column chromatography (SiO₂, ethyl acetate/heptane 50/50) yielding the title compound (430 mg, 50% yield) as a white foam.

¹H NMR (300 MHz, CDCl₃): δ=1.18 (s, 9H), 3.45 (br.s, 3H), 3.70 (dd, 1H), 3.80 (dd, 1H), 4.36 (dd, 1H), 7.18 (br.s, 1H), 7.29 (m, 1H), 7.39 (m, 4H), 7.73 (br.d, 1H), 7.90 (br.s, 1H), 8.34 (br.s, 1H); HPLC-MS (Method A): m/z=429 (M+H)⁺; Rt=3.88 min.

### Example 11

### [Methyl-phenyl-carbamic acid 5-(3-tert-butyl-thioureido)-pyridin-2-yl ester]

A solution of tert-butylamine (146 mg, 2.0 mmol) in dichloromethane (5 mL) was added to a solution of methyl-phenyl-carbamic acid 5-isothiocyanato-pyridin-2-yl ester (0.57 g, 2.0 mmol) in dichloromethane (5 mL). The solution was stirred for 1 hour at room temperature. Without further work-up, the reaction mixture was purified by flash column chromatography (SiO₂, ethyl acetate/heptane 50/50), yielding the title compound (542 mg, 76% yield) as a white solid.

¹H NMR (300 MHz, CDCl₃): δ=1.43 (s, 9H), 3.43+3.50 (2×br.s, 3H), 6.27+6.41 (2×br.s, 1H), 6.96+7.06 (2×br.s, 1H), 7.25-7.44 (m, 5H), 7.68+7.78 (2×br.s, 2H), 8.00 (br.s, 1H); HPLC-MS (Method A): m/z=359 (M+H)⁺; Rt=3.74 min.

### Example 12

### [Methyl-phenyl-carbamic acid 5-[3-(2,2-dimethyl-propyl)-thioureido]-pyridin-2-yl ester]

A solution of 2,2-dimethylpropylamine (174 mg, 2.0 mmol) in dichloromethane (5 mL) was added to a solution of methyl-phenyl-carbamic acid 5-isothiocyanato-pyridin-2-yl ester (0.57 g, 2.0 mmol) in dichloromethane (5 mL). The solution was stirred for 1 hour at room temperature. Without further work-up, the reaction mixture was purified by flash column chromatography (SiO₂, ethyl acetate/heptane 50/50), yielding the title compound (674 mg, 90% yield) as a white solid.

¹H NMR (300 MHz, CDCl₃): δ=0.89 (s, 9H), 3.33-3.60 (br.m, 5H), 6.59+6.70 (2×br.s, 1H), 6.88+7.01 (2×br.s, 1H), 7.26-7.45 (m, 5H), 7.75-8.26 (5×br.s, 3H); HPLC-MS (Method A): m/z=373 (M+H)⁺; Rt=4.10 min.

### Example 13

### [Methyl-phenyl-carbamic acid 5-(3-isopropyl-thioureido)-pyridin-2-yl ester]

A solution of isopropylamine (118 mg, 2.0 mmol) in dichloromethane (5 mL) was added to a solution of methyl-phenyl-carbamic acid 5-isothiocyanato-pyridin-2-yl ester (0.57 g, 2.0 mmol) in dichloromethane (5 mL). The solution was stirred for 1 hour at room temperature. Without further work-up, the product was purified by flash column chromatography (SiO₂, ethyl acetate/heptane (50/50), yielding the title compound (560 mg, 81% yield) as a white solid.

¹H NMR (300 MHz, CDCl₃): δ=1.08 (d, 6H), 3.45+3.52 (2×br.s, 3H), 4.47 (octet, 1H), 6.38+6.60 (2×br.s, 1H), 6.90+7.03 (2×br.s, 1H), 7.26-7.50 (m, 5H), 7.68-8.02 (3×br.s, 3H); HPLC-MS (Method A): m/z=345 (M+H)⁺; Rt=3.35 min.

### Example 14

### [Methyl-phenyl-carbamic acid 5-(3,3-diethyl-thioureido)-pyridin-2-yl ester]

A solution of diethylamine (146 mg, 2.0 mmol) in dichloromethane (5 mL) was added to a solution of methyl-phenyl-carbamic acid 5-isothiocyanato-pyridin-2-yl ester (0.57 g, 2.0 mmol) in dichloromethane (5 mL). The solution was stirred for 1 hour at room temperature. Without further work-up, the product was purified by flash column chromatography (SiO₂, ethyl acetate/heptane (50/50), yielding the title compound (196 mg, 27% yield) as a white solid.

¹H NMR (300 MHz, CDCl₃): δ=1.27 (t, 6H), 3.42 (br.s, 3H), 3.75 (q, 4H), 6.91 (br.s, 1H), 7.26 (m, 1H), 7.37 (m, 5H), 7.78 (br.d, 1H), 8.12 (br.s, 1H); HPLC-MS (Method A): m/z=359 (M+H)⁺; Rt=3.44 min.

### Example 15

### [Methyl-phenyl-carbamic acid 5-(3-cyclohexyl-thioureido)-pyridin-2-yl ester]

A solution of cyclohexylamine (198 mg, 2.0 mmol) in dichloromethane (5 mL) was added to a solution of methyl-phenyl-carbamic acid 5-isothiocyanato-pyridin-2-yl ester (0.57 g, 2.0 mmol) in dichloromethane (5 mL). The solution was stirred for 1 hour at room temperature. Without further work-up, the product was purified by flash column chromatography (SiO₂, ethyl acetate/heptane (60/40), yielding the title compound (554 mg, 72% yield) as a white solid.

¹H NMR (300 MHz, CDCl₃): δ=0.88 (m, 2H), 1.07 (m, 1H), 1.30 (m, 2H), 1.62 (m, 3H), 1.94 (d, 2H), 3.44+3.54 (2×br.s, 3H), 4.11 (m, 1H), 6.39+6.62 (2×br.s, 1H), 6.90+7.01 (2×br.s, 1H), 7.23-7.50 (m, 5H), 7.63-8.04 (br.m, 3H); HPLC-MS (Method A): m/z=385 (M+H)⁺; Rt=3.97 min.

### Example 16

### [Methyl-phenyl-carbamic acid 5-(3-butyl-thioureido)-pyridin-2-yl ester]

A solution of n-butylamine (146 mg, 2.0 mmol) in dichloromethane (5 mL) was added to a solution of methyl-phenyl-carbamic acid 5-isothiocyanato-pyridin-2-yl ester (0.57 g, 2.0 mmol) in dichloromethane (5 mL). The solution was stirred for 1 hour at room temperature. Without further work-up, the product was purified by flash column chromatography (SiO₂, ethyl acetate/heptane (70/30), yielding the title compound (627 mg, 87% yield) as a white solid.

¹H NMR (300 MHz, CDCl₃): δ=0.90 (t, 3H), 1.20-1.47 (m, 4H), 3.42+3.53 (2×br.s, 5H), 6.51+6.80 (2×br.s, 1H), 6.91+7.04 (2×br.s, 1H), 7.38-7.50 (m, 5H), 7.65-8.17 (3×br.s, 3H); HPLC-MS (Method A): m/z=359 (M+H)⁺; Rt=3.70 min.

### Example 17

### [Methyl-phenyl-carbamic acid 5-(3-isobutyl-thioureido)-pyridin-2-yl ester]

A solution of isobutylamine (146 mg, 2.0 mmol) in dichloromethane (5 mL) was added to a solution of methyl-phenyl-carbamic acid 5-isothiocyanato-pyridin-2-yl ester (0.57 g, 2.0 mmol) in dichloromethane (5 mL). The solution was stirred for 1 hour at room temperature. Without further work-up, the product was purified by flash column chromatography (SiO₂, ethyl acetate/heptane (70/30), yielding the title compound (621 mg, 87% yield) as a white solid.

¹H NMR (300 MHz, CDCl₃): δ=0.84 (d, 6H), 1.74 (br.s, 1H), 3.29 (br.s, 2H), 3.42+3.53 (2×br.s, 3H), 6.53+6.82 (2×br.s, 1H), 6.94+7.06 (2×br.s, 1H), 7.27-7.50 (m, 5H), 7.70-8.14 (br.m, 3H); HPLC-MS (Method A): m/z=359 (M+H)⁺; Rt=3.67 min.

### Example 18

### [Methyl-phenyl-carbamic acid 5-(4-cyano-benzoylamino)-pyridin-2-yl ester]

A solution of methyl-phenyl-carbamic acid 5-amino-pyridin-2-yl ester (122 mg, 0.5 mmol), 4-cyanobenzoyl chloride (91 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (125 mg, 67% yield) as a white solid.

¹H NMR (300 MHz, CDCl₃): δ=3.39+3.53 (2×br.s, 3H), 6.88 (br.s, 1H), 7.27-7.48 (m, 5H), 7.63 (d, 2H), 7.98 (d, 3H), 8.34 (d, 1H), 8.81+9.08 (2×br.s, 1H); HPLC-MS (Method A): m/z=373 (M+H)⁺; Rt=3.50 min.

### Example 19

### [Methyl-phenyl-carbamic acid 5-(2-methyl-benzoylamino)-pyridin-2-yl ester]

A solution of methyl-phenyl-carbamic acid 5-amino-pyridin-2-yl ester (122 mg, 0.5 mmol), 2-methylbenzoyl chloride (85 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (361 mg, 66% yield) as an oil.

¹H NMR (300 MHz, CDCl₃): δ=2.45 (s, 3H), 3.21+3.42 (2×br.s, 3H), 6.90 (br.s, 1H), 7.12-7.39 (m, 8H), 7.45 (d, 1H), 8.08 (dd, 1H), 8.32 (s, 1H), 8.54 (s, 1H); HPLC-MS (Method A): m/z=362 (M+H)⁺; Rt=3.62 min.

### Example 20

### [Methyl-phenyl-carbamic acid 5-(4-fluoro-benzoylamino)-pyridin-2-yl ester]

A solution of methyl-phenyl-carbamic acid 5-amino-pyridin-2-yl ester (122 mg, 0.5 mmol), 4-fluorobenzoyl chloride (87 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (74 mg, 41% yield) as a white solid.

¹H NMR (300 MHz, CDCl₃): δ=3.41 (br.s, 3H), 6.91 (br.s, 1H), 7.07 (m, 2H), 7.27 (m, 1H), 7.32-7.44 (m, 4H), 7.90 (m, 2H), 8.05 (dd, 1H), 8.33 (d, 1H), 8.50+8.65 (2×br.s, 1H); HPLC-MS (Method A): m/z=366 (M+H)⁺; Rt=3.61 min.

### Example 21

### [Methyl-phenyl-carbamic acid 5-(3-methoxy-benzoylamino)-pyridin-2-yl ester]

A solution of methyl-phenyl-carbamic acid 5-amino-pyridin-2-yl ester (122 mg, 0.5 mmol), 3-methoxybenzoyl chloride (94 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (210 mg, 111% yield) as an oil, which solidified upon standing

¹H NMR (300 MHz, CDCl₃): δ=3.40 (br.s, 3H), 3.82 (s, 3H), 6.89 (br.s, 1H), 7.05 (dd, 1H), 7.21-7.49 (m, 8H), 8.06 (br.d, 1H), 8.39 (d, 1H), 8.87 (br.s, 1H); HPLC-MS (Method A): m/z=378 (M+H)⁺; Rt=3.62 min.

### Example 22

### [Methyl-phenyl-carbamic acid 5-(2-methoxy-benzoylamino)-pyridin-2-yl ester]

A solution of methyl-phenyl-carbamic acid 5-amino-pyridin-2-yl ester (122 mg, 0.5 mmol), 2-methoxybenzoyl chloride (94 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (109 mg, 57% yield) as a white solid.

¹H NMR (300 MHz, CDCl₃): δ=3.44 (br.s, 3H), 4.06 (s, 3H), 7.02 (d, 1H), 7.06 (br.s, 1H), 7.12 (t, 1H), 7.26 (m, 1H), 7.39 (d, 4H), 7.51 (m, 1H), 8.36 (dd, 1H), 8.37 (m, 1H), 8.43 (s, 1H), 9.87 (s, 1H); HPLC-MS (Method A): m/z=378 (M+H)⁺; Rt=3.72 min.

### Example 23

### [Methyl-phenyl-carbamic acid 5-(3,4-dichloro-benzoylamino)-pyridin-2-yl ester]

A solution of methyl-phenyl-carbamic acid 5-amino-pyridin-2-yl ester (122 mg, 0.5 mmol), 3,4-dichlorobenzoyl chloride (91 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (130 mg, 63% yield) as a white solid.

¹H NMR (300 MHz, CDCl₃): δ=3.41 (br.s, 3H), 6.81+6.92 (2×br.s, 1H), 7.27 (m, 1H), 7.38 (m, 4H), 7.44 (d, 1H), 7.72 (dd, 1H), 7.92 (br.s, 1H), 8.03 (d, 1H), 8.30 (s, 1H), 8.90 (br.s, 1H); HPLC-MS (Method A): m/z=416 (M+H)⁺; Rt=4.30 min.

### Example 24

### [Methyl-phenyl-carbamic acid 5-(4-methyl-benzoylamino)-pyridin-2-yl ester]

A solution of methyl-phenyl-carbamic acid 5-amino-pyridin-2-yl ester (122 mg, 0.5 mmol), 4-methylbenzoyl chloride (85 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography, yielding the title compound (157 mg, 83% yield) as a white solid.

¹H NMR (300 MHz, CDCl₃): δ=2.40 (s, 3H), 3.41 (br.s, 3H), 6.90 (br.s, 1H), 7.18 (d, 2H), 7.25 (m, 1H), 7.36 (m, 4H), 7.78 (d, 2H), 8.10 (dd, 1H), 8.35 (d, 1H), 8.63 (br.s, 1H); HPLC-MS (Method A): m/z=362 (M+H)⁺; Rt=3.75 min.

### Example 25

### [Methyl-phenyl-carbamic acid 5-(3-bromo-benzoylamino)-pyridin-2-yl ester]

A solution of methyl-phenyl-carbamic acid 5-amino-pyridin-2-yl ester (122 mg, 0.5 mmol), 3-bromobenzoyl chloride (121 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (105 mg, 49% yield) as a white solid.

¹H NMR (300 MHz, CDCl₃): δ=3.43 (br.s, 3H), 6.84 (br.s, 1H), 7.27 (m, 2H), 7.37 (m, 4H), 7.63 (d, 1H), 7.83 (d, 1H), 7.97 (br.s, 1H), 8.05 (s, 1H), 8.32 (s, 1H), 8.90 (s, 1H); HPLC-MS (Method A): m/z=428 (M+H)⁺; Rt=4.03 min.

### Example 26

### [Methyl-phenyl-carbamic acid 5-(3-cyano-benzoylamino)-pyridin-2-yl ester]

A solution of methyl-phenyl-carbamic acid 5-amino-pyridin-2-yl ester (122 mg, 0.5 mmol), 3-cyanobenzoyl chloride (91 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (151 mg, 81% yield) as a white solid.

¹H NMR (300 MHz, CDCl₃): =3.45 (br.s, 3H), 6.80+6.95 (2×br.s, 1H), 7.28 (m, 1H), 7.39 (m, 4H), 7.52 (t, 1H), 7.80 (d, 1H), 7.90 (br.s, 1H), 8.18 (d, 1H), 8.28 (s, 1H), 8.32 (s, 1H), 9.13 (s, 1H); HPLC-MS (Method A): m/z=373 (M+H)⁺; Rt=3.52 min.

### Example 27

### [Methyl-phenyl-carbamic acid 5-(2-trifluoromethoxy-benzoylamino)-pyridin-2-yl ester]

A solution of methyl-phenyl-carbamic acid 5-amino-pyridin-2-yl ester (122 mg, 0.5 mmol), 2-trifluoromethoxybenzoyl chloride (124 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (200 mg, 93% yield) as an oil, which solidified upon standing.

¹H NMR (300 MHz, CDCl₃): δ=3.37 (br.s, 3H), 7.01 (br.s, 1H), 7.26 (m, 1H), 7.28-7.44 (m, 6H), 7.54 (m, 1H), 7.98 (dd, 1H), 8.20 (dd, 1H), 8.40 (s, 1H), 8.60 (s, 1H); HPLC-MS (Method A): m/z=432 (M+H)⁺; Rt=3.94 min.

### Example 28

### [Methyl-phenyl-carbamic acid 5-(2-fluoro-3-trifluoromethyl-benzoylamino)-pyridin-2-yl ester]

A solution of methyl-phenyl-carbamic acid 5-amino-pyridin-2-yl ester (122 mg, 0.5 mmol), 2-fluoro-3(trifluoromethyl)benzoyl chloride (125 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (150 mg, 69% yield) as a white solid.

¹H NMR (300 MHz, CDCl₃): δ=3.37 (br.s, 3H), 7.00 (br.s, 1H), 7.23 (m, 1H), 7.33 (m, 5H), 7.79 (t, 1H), 8.18 (m, 2H), 8.40 (s, 1H), 8.67 (d, 1H); HPLC-MS (Method A): m/z=434 (M+H)⁺; Rt=4.12 min.

### Example 29

### [Methyl-phenyl-carbamic acid 5-(3-trifluoromethyl-benzoylamino)-pyridin-2-yl ester]

A solution of methyl-phenyl-carbamic acid 5-amino-pyridin-2-yl ester (122 mg, 0.5 mmol), 3-(trifluoromethyl)benzoyl chloride (115 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (171 mg, 82% yield) as a white solid.

¹H NMR (300 MHz, CDCl₃): δ=3.37+3.51 (2×br.s, 3H), 6.80+6.95 (2×br.s, 1H), 7.18-7.46 (m, 5H), 7.53 (t, 1H), 7.78 (d, 1H), 8.00 (d, 1H), 8.11 (d, 1H), 8.21 (s, 1H), 8.30 (br.s, 1H), 9.08 (s, 1H); HPLC-MS (Method A): m/z=416 (M+H)⁺; Rt=4.15 min.

### Example 30

### [Methyl-phenyl-carbamic acid 5-(3,4-difluoro-benzoylamino)-pyridin-2-yl ester]

A solution of methyl-phenyl-carbamic acid 5-amino-pyridin-2-yl ester (122 mg, 0.5 mmol), 3,4-difluorobenzoyl chloride (91 mg, 0.55 mmol) and triethylamine (56 mg, 0.55 mmol) in acetonitrile (2 mL) was stirred at room temperature for 1 hour. Dichloromethane (3 mL) was added to get a clear solution. The product was purified by flash column chromatography (SiO₂), yielding the title compound (169 mg, 88% yield) as a white solid.

¹H NMR (300 MHz, CDCl₃): δ=3.43 (br.s, 3H), 6.81 (br.s, 1H), 7.15 (dd, 1H), 7.30 (m, 1H), 7.39 (m, 4H), 7.68 (m, 1H), 7.82 (m, 2H), 8.30 (d, 1H), 8.90 (br.s, 1H); HPLC-MS (Method A): m/z=384 (M+H)⁺; Rt=3.47 min.

### Example 31

### [Methyl-phenyl-carbamic acid 5-(3-dimethylamino-benzoylamino)-pyridin-2-yl ester]

A solution of methyl-phenyl-carbamic acid 5-amino-pyridin-2-yl ester (242 mg, 1.0 mmol), 3-dimethylaminobenzoyl chloride hydrochloride (242 mg, 1.1 mmol) and triethylamine (0.31 mL, 2.2 mmol) in dichloromethane (10 mL) was stirred at room temperature for 1 hour. The product was purified by flash column chromatography (SiO₂, gradient of 0-15% ethyl acetate in dichloromethane) yielding the title compound (5.5 mg, 1% yield).

¹H NMR (300 MHz, CDCl₃): δ=3.01 (s, 6H), 3.42 (br.s, 3H), 6.89 (dd, 1H), 7.02 (br.s, 1H), 7.10 (d, 1H), 7.22-7.3 (m, 3H), 7.40 (m, 4H), 8.13 (br.s, 1H), 8.25 (dd, 1H), 8.41 (br.s, 1H); HPLC-MS (Method A): m/z=391 (M+H)⁺; Rt=2.94 min.

### Example 32

### [Methyl-phenyl-carbamic acid 5-(3-dimethylamino-benzoylamino)-pyridin-2-yl ester]

Phosgene (5 mL, 20% in toluene) was added to a stirred suspension of 6′-hydroxy-4,4-dimethyl-4,5-dihydro-3H-[1,3′]bipyridinyl-2,6-dione (0.47 g, 2.00 mmol) and triethylamine (0.29 mL, 2.00 mmol) in dichloromethane (10 mL). After stirring for 0.75 h at room temperature, the solvent and excess phosgene were evaporated under reduced pressure, yielding a white solid. The residue was dissolved in dichloromethane (10 mL) followed by the addition of 1,4-diazabicyclo[2.2.2]octane (224 mg, 2.00 mmol) and 2-methoxy-N-methylaniline (0.27 g, 2.00 mmol) and stirring was continued for 0.75 hours at room temperature. The solution was extracted with water and evaporated in vacuo. The residue was purified by flash column chromatography (SiO₂, gradient of 0-15% ethyl acetate in dichloromethane), yielding the title compound (260 mg, 33% yield) as a white solid.

¹H NMR (300 MHz, CDCl₃): δ=1.19 (s, 6H), 2.66 (s, 4H), 3.29+3.40 (2×s, 3H), 3.86 (s, 3H), 6.94 (m, 2H), 7.04-7.54 (m, 4H), 8.03+8.12 (d+s, 1H); HPLC-MS (Method A): m/z=398 (M+H)⁺; Rt=3.62 min.

### Example 33

### [Methyl-phenyl-carbamic acid 5-(4-nitro-benzoylamino)-pyridin-2-yl ester]

A solution of methyl-phenyl-carbamic acid 5-amino-pyridin-2-yl ester (0.97 g, 4.00 mmol), 4-nitrobenzoyl chloride (0.78 g, 4.20 mmol) and triethylamine (0.59 mL, 4.20 mmol) in dichloromethane (10 mL) was stirred at room temperature for 3 days. The solution was extracted with water and evaporated in vacuo. The residue was purified by flash column chromatography (SiO₂, gradient of 40-80% ethyl acetate in heptane) yielding the title compound (1.51 g, 96% yield) as a white solid.

¹H NMR (300 MHz, CDCl₃): δ 3.42+3.53 (2×br.s, 3H), 6.93 (br.s, 1H), 7.27-7.48 (m, 5H), 8.00 (d, 1H), 8.03 (dd, 1H+ d, 2H), 8.20 (d, 2H), 8.38 (s, 1H), 8.68+9.03 (2×br.s, 1H); HPLC-MS (Method A): m/z=393 (M+H)⁺; Rt=3.67 min.

### Example 34

### [Methyl-phenyl-carbamic acid 5-(4-amino-benzoylamino)-pyridin-2-yl ester]

A suspension of methyl-phenyl-carbamic acid 5-(4-nitro-benzoylamino)-pyridin-2-yl ester (1.50 g, 3.82 mmol) in ethyl acetate was hydrogenated in a Parr apparatus at 40 psi hydrogen pressure over night. The solution was filtered over a short pad of Celite and washed thoroughly with dichloromethane. The filtrate was evaporated in vacuo yielding the title compound (495 mg, 36% yield) as a slightly yellow solid.

¹H NMR (300 MHz, DMSO-d₆): &=3.33 (s, 3H), 5.80 (s, 2H), 6.60 (d, 2H), 7.17 (d, 1H), 7.29 (m, 1H), 7.44 (m, 4H), 7.72 (d, 2H), 8.22 (dd, 1H), 8.62 (d, 1H), 10.00 (s, 1H); HPLC-MS (Method A): m/z=363 (M+H)⁺; Rt=2.83 min.

### Example 35

### [Methyl-phenyl-carbamic acid 5-(3-tert-butyl-ureido)-pyridin-2-yl ester]

A solution of methyl-phenyl-carbamic acid 5-amino-pyridin-2-yl ester (243 mg, 1.00 mmol) and tert-butylisocyanate (0.114 mL, 1.00 mmol) in dichloromethane (5 mL) was stirred at room temperature. The solvent was replaced by tetrahydrofuran and the solution was heated at 40° C. for 3 days. Evaporation of the solvent and purification of the residue by flash column chromatography (SiO₂, gradient of 50-70% of ethyl acetate in heptane) yielded the title compound (119 mg, 35% yield) as a white solid.

¹H NMR (300 MHz, CDCl₃): δ 1.24 (s, 9H), 3.43 (br.s, 3H), 5.42 (br.s, 1H), 6.85 (br.s, 1H), 7.26 (m, 1H), 7.37 (m, 5H), 7.82 (br.s, 1H), 7.89 (dd, 1H); HPLC-MS (Method A): m/z=343 (M+H)⁺; Rt=3.31 min.

### Example 36

### [Methyl-phenyl-carbamic acid 5-(4-formyl-benzoylamino)-pyridin-2-yl ester]

4-Formylbenzoyl chloride (1.56 g, 9.28 mmol) was added to a stirred solution of methyl-phenyl-carbamic acid 5-amino-pyridin-2-yl ester (2.15 g, 8.84 mmol) and triethylamine (1.3 mL, 9.28 mmol). After 0.5 hours stirring at room temperature, the solvent was evaporated and the residue purified by flash column chromatography (SiO₂, ethyl acetate/heptane 70/30), yielding the title compound (2.71 g, 82% yield).

¹H NMR (300 MHz, CDCl₃): δ 3.39+3.52 (2×br.s, 3H), 6.87 (br.s, 1H), 7.23-7.44 (m, 5H), 7.89 (d, 2H), 8.01 (m, 3H), 8.94+9.10 (2×br.s, 1H), 10.10 (s, 1H); HPLC-MS (Method A): m/z=476 (M+H)⁺; Rt=3.36 min.

### Example 37

### [Methyl-phenyl-carbamic acid 5-(4-morpholin-4-ylmethyl-benzoylamino)-pyridin-2-yl ester]

Morpholine (0.144 mL, 1.65 mmol) was added to a stirred suspension of methyl-phenyl-carbamic acid 5-(4-formyl-benzoylamino)-pyridin-2-yl ester (0.56 g, 1.5 mmol) in methanol (15 mL). The solution was warmed carefully to get the starting material into solution. After stirring for 0.5 hours at room temperature sodium cyanoborohydride (104 mg, 1.65 mmol) was added and stirring was continued at room temperature overnight. The solvent was evaporated and the residue purified by flash column chromatography (SiO₂), yielding the title compound (38 mg, 6% yield).

¹H NMR (300 MHz, CDCl₃): δ 2.45 (m, 4H), 3.41 (br.s, 3H), 3.53 (s, 2H), 3.71 (m, 4H), 6.91 (br.s, 1H), 7.27 (m, 1H), 7.38 (m, 6H), 7.84 (d, 2H), 8.09 (dd, 1H), 8.38 (d, 1H), 8.75 (br.s, 1H); HPLC-MS (Method A): m/z=447 (M+H)⁺; Rt=2.43 min.

### Example 38

### [Methyl-phenyl-carbamic acid 5-(4-hydroxymethyl-benzoylamino)-pyridin-2-yl ester]

The title compound (16.4 mg, 2% yield) was isolated by flash column chromatography (SiO₂) from the reaction mixture as described in Example 37.

¹H NMR (300 MHz, DMSO-d₆): (=3.37 (br.s, 3H), 4.61 (d, 2H), 5.35 (t, 1H), 7.22 (d, 1H), 7.30 (m, 1H), 7.40-7.51 (m, 6H), 7.97 (d, 2H), 8.27 (dd, 1H), 8.68 (d, 1H), 10.43 (s, 1H); HPLC-MS (Method A): m/z=378 (M+H)⁺; Rt=2.94 min.

### Example 39

### [Methyl-phenyl-carbamic acid 5-(4-chloromethyl-benzoylamino)-pyridin-2-yl ester]

A solution of 4-(chloromethyl)benzoyl chloride (8.39 g, 44.36 mmol) in dichloromethane (50 mL) was added dropwise to a stirred solution of methyl-phenyl-carbamic acid 5-amino-pyridin-2-yl ester (10.79 g, 44.36 mmol) and triethylamine (6.2 mL, 44.36 mmol) in dichloro-methane (50 mL) with a some dimethylformamide. After stirring for 1 hour at room temperature, the solution was extracted with water, dried over sodium sulphate and evaporated in vacuo, yielding the title compound (14.8 g, 84% yield) as an off-white solid.

¹H NMR (300 MHz, CDCl₃): δ=3.37 (br.s, 3H), 4.85 (s, 2H), 7.24 (d, 1H), 7.30 (m, 1H), 7.46 (m, 4H), 7.62 (d, 2H), 8.00 (d, 2H), 8.28 (dd, 1H), 8.69 (d, 1H).; HPLC-MS (Method A): m/z=396 (M+H)⁺; Rt=3.83 min.

### Example 40

### [Methyl-phenyl-carbamic acid 5-(4-piperidin-1-ylmethyl-benzoylamino)-pyridin-2-yl ester]

Piperidine (0.17 g, 1.00 mmol) was added to a stirred solution of methyl-phenyl-carbamic acid 5-(4-chloromethyl-benzoylamino)-pyridin-2-yl ester (0.40 g, 1.00 mmol) in dimethylformamide (5 mL), followed by a catalytic amount of sodium iodide. After stirring for 18 hours at room temperature water was added slowly to the reaction mixture. The solid material was isolated by suction and dried in a vacuum oven, yielding the title compound (397 mg, 89% yield) as an off-white solid.

¹H NMR (300 MHz, CDCl₃): &=1.46 (m, 2H), 1.58 (m, 4H), 2.38 (m, 4H), 3.41 (br.s, 3H), 3.51 (s, 2H), 6.96 (br.s, 1H), 7.27 (m, 1H), 7.40 (m, 6H), 7.84 (d, 2H), 8.18 (dd, 1H), 8.39 (d, 1H+br.s, 1H); HPLC-MS (Method A): m/z=445 (M+H)⁺; Rt=2.57 min.

### Example 41

### [Methyl-phenyl-carbamic acid 5-[4-(4-methyl-piperidin-1-ylmethyl)-benzoylamino]-pyridin-2-yl ester]

4-Methylpiperidine (0.20 g, 1.00 mmol) was added to a stirred solution of methyl-phenyl-carbamic acid 5-(4-chloromethyl-benzoylamino)-pyridin-2-yl ester (0.40 g, 1.00 mmol) in dimethylformamide (5 mL), followed by a catalytic amount of sodium iodide. After stirring for 18 hours at room temperature, water was added slowly to the reaction mixture. The solid material was isolated by suction and dried in a vacuum oven, yielding the title compound (393 mg, 86% yield) as an off-white solid.

¹H NMR (300 MHz, CDCl₃): δ=0.91 (d, 3H), 1.28 (m, 3H), 1.60 (m, 2H), 2.00 (m, 2H), 2.84 (m, 2H), 3.40 (br.s, 3H), 3.53 (s, 3H), 6.97 (br.s, 1H), 7.27 (m, 1H), 7.39 (m, 6H), 7.85 (d, 2H), 8.20 (dd, 1H), 8.40 (d, 1H), 8.47 (br.s, 1H); HPLC-MS (Method A): m/z=459 (M+H)⁺; Rt=2.74 min.

### Example 42

### [Methyl-phenyl-carbamic acid 5-[4-(2-ethyl-piperidin-1-ylmethyl)-benzoylamino]-pyridin-2-yl ester]

2-Ethylpiperidine (0.23 g, 1.00 mmol) was added to a stirred solution of methyl-phenyl-carbamic acid 5-(4-chloromethyl-benzoylamino)-pyridin-2-yl ester (0.40 g, 1.00 mmol) in dimethylformamide (5 mL), followed by a catalytic amount of sodium iodide. After stirring for 18 hours at room temperature, water was added slowly to the reaction mixture. The solvent was decanted and the residue was dried in a vacuum oven, yielding the title compound (430 mg, 91% yield) as an off-white solid.

¹H NMR (300 MHz, CDCl₃): δ=0.92 (t, 3H), 1.32 (m, 1H), 1.38-1.75 (m, 7H), 2.03 (m, 1H), 2.27 (m, 1H), 2.70 (m, 1H), 3.26 (d, 1H), 3.41 (br.s, 3H), 4.02 (d, 1H), 6.95 (br.s, 1H), 7.26 (m, 1H), 7.38 (m, 4H), 7.43 (d, 2H), 8.19 (dd, 1H), 8.42 (d, 1H), 8.62 (br.s, 1H); HPLC-MS (Method A): m/z=473 (M+H)⁺; Rt=2.77 min.

### Example 43

### [Methyl-phenyl-carbamic acid 5-[4-(2-methyl-piperidin-1-ylmethyl)-benzoylamino]-pyridin-2-yl ester]

2-Methylpiperidine (0.20 g, 1.00 mmol) was added to a stirred solution of methyl-phenyl-carbamic acid 5-(4-chloromethyl-benzoylamino)-pyridin-2-yl ester (0.40 g, 1.00 mmol) in dimethylformamide (5 mL), followed by a catalytic amount of sodium iodide. After stirring for 18 hours at room temperature, water was added slowly to the reaction mixture. The solid material was isolated by suction and dried in a vacuum oven, yielding the title compound (293 mg, 64% yield) as an off-white solid.

¹H NMR (300 MHz, CDCl₃): δ=1.16 (d, 3H), 1.21-1.72 (m, 6H), 1.98 (m, 1H), 2.34 (m, 1H), 2.69 (m, 1H), 3.24 (d, 1H), 3.45 (br.s, 3H), 4.04 (d, 1H), 7.00 (br.s, 1H), 7.27 (m, 1H), 7.40 (m, 6H), 7.82 (d, 2H), 8.21 (dd, 1H+br.s, 1H), 8.38 (d, 1H); HPLC-MS (Method A): m/z=459 (M+H)⁺; Rt=2.64 min.

### Example 44

### [Methyl-phenyl-carbamic acid 5-[4-(3-methyl-piperidin-1-ylmethyl)-benzoylamino]-pyridin-2-yl ester]

3-Methylpiperidine (0.20 g, 1.00 mmol) was added to a stirred solution of methyl-phenyl-carbamic acid 5-(4-chloromethyl-benzoylamino)-pyridin-2-yl ester (0.40 g, 1.00 mmol) in dimethylformamide (5 mL), followed by a catalytic amount of sodium iodide. After stirring for 18 hours at room temperature, water was added slowly to the reaction mixture. The solvent was decanted and the residue was dried in a vacuum oven, yielding the title compound (292 mg, 64% yield) as an off-white solid.

¹H NMR (300 MHz, CDCl₃): δ=0.83 (d, 3H+m, 1H), 1.48-1.77 (m, 5H), 1.89 (dt, 1H), 2.78 (m, 2H), 3.40 (br.s, 3H), 3.51 (s, 2H), 6.90 (br.s, 1H), 7.27 (m, 1H), 7.38 (m, 6H), 7.85 (d, 2H), 8.10 (dd, 1H), 8.38 (d, 1H), 8.69 (br.s, 1H).; HPLC-MS (Method A): m/z=459 (M+H)⁺; Rt=2.71 min.

### Example 45

### [Methyl-phenyl-carbamic acid 5-(4-dimethylaminomethyl-benzoylamino)-pyridin-2-yl ester]

Dimethylamine hydrochloride (82 mg, 1.00 mmol) and triethylamine (0.28 mL, 2.00 mmol) were added to a stirred solution of methyl-phenyl-carbamic acid 5-(4-chloromethyl-benzoylamino)-pyridin-2-yl ester (0.40 g, 1.00 mmol) in N,N-dimethylformamide (5 mL), followed by a catalytic amount of sodium iodide. The reaction mixture was stirred for 18 hours at room temperature. Water was added slowly and decanted. The residue was purified by preparative HPLC, yielding the title compound (165 mg, 32% yield) as the TFA-salt.

¹H NMR (300 MHz, CDCl₃): δ=2.60 (s, 6H), 3.40 (br.s, 3H), 4.06 (s, 2H), 6.99 (br.s, 1H), 7.18-7.42 (m, 7H), 7.82 (d, 2H), 8.19 (d, 1H), 8.54 (s, 1H), 9.81 (br.s, 1H), 11.74 (br.s, 1H); HPLC-MS (Method A): m/z=461 (M+H)⁺; Rt=2.82 min.

### Example 46

### [Methyl-phenyl-carbamic acid 5-(4-diethylaminomethyl-benzoylamino)-pyridin-2-yl ester]

Diethylamine (0.15 g, 2.00 mmol) was added to a stirred solution of methyl-phenyl-carbamic acid 5-(4-chloromethyl-benzoylamino)-pyridin-2-yl ester (0.40 g, 1.00 mmol) in N,N-dimethylformamide (5 mL), followed by a catalytic amount of sodium iodide. The reaction mixture was stirred for 18 hours at room temperature. Water was added slowly, the solid material was isolated by suction and dried in a vacuum oven, yielding the title compound (404 mg, 93% yield) as a white solid.

¹H NMR (300 MHz, CDCl₃): δ=1.06 (t, 6H), 2.53 (q, 4H), 3.42 (br.s, 3H), 3.61 (s, 2H), 6.97 (br.s, 1H), 7.27 (m, 1H), 7.39 (m, 4H), 7.45 (d, 2H), 7.83 (d, 2H), 8.18 (dd, 1H), 8.38 (d, 1H), 8.42 (br.s, 1H); HPLC-MS (Method A): m/z=433 (M+H)⁺; Rt=2.58 min.

### Example 47

### [Methyl-phenyl-carbamic acid 5-(4-pyrrolidin-1-ylmethyl-benzoylamino)-pyridin-2-yl ester]

Using the procedure as described in Example 46 and starting from pyrrolidine (0.14 g, 2.00 mmol) and methyl-phenyl-carbamic acid 5-(4-chloromethyl-benzoylamino)-pyridin-2-yl ester (0.40 g, 1.00 mmol) yielded the title compound (357 mg, 83% yield) as a white solid.

¹H NMR (300 MHz, CDCl₃): δ=1.80 (m, 4H), 2.51 (m, 4H), 3.42 (br.s, 3H), 3.67 (s, 2H), 6.94 (br.s, 1H), 7.27 (m, 1H), 7.40 (m, 6H), 6.84 (d, 2H), 8.15 (dd, 1H), 8.37 (d, 1H), 8.52 (br.s, 1H); HPLC-MS (Method A): m/z=431 (M+H)⁺; Rt=2.56 min.

### Example 48

### [Methyl-phenyl-carbamic acid 5-(4-dipropylaminomethyl-benzoylamino)-pyridin-2-yl ester]

Using the procedure as described in Example 46 and starting from di-n-propylamine (0.20 g, 2.00 mmol) and methyl-phenyl-carbamic acid 5-(4-chloromethyl-benzoylamino)-pyridin-2-yl ester (0.40 g, 1.00 mmol) yielded the title compound (402 mg, 87% yield) as a white solid.

¹H NMR (300 MHz, CDCl₃): δ=0.88 (t, 6H), 1.48 (sextet, 4H), 2.37 (t, 4H), 3.42 (br.s, 3H), 3.60 (s, 2H), 6.94 (br.s, 1H), 7.27 (m, 1H), 7.38 (m, 4H), 7.42 (d, 2H), 7.83 (d, 2H), 8.15 (dd, 1H), 8.38 (d, 1H), 8.47 (br.s, 1H); HPLC-MS (Method A): m/z=461 (M+H)⁺; Rt=2.82 min.

### Example 49

### [Methyl-phenyl-carbamic acid 5-(4-pyrrolidin-1-ylmethyl-benzoylamino)-pyridin-2-yl ester]

Using the procedure as described in Example 46 and starting from 4,4-dimethylpiperidine hydrochloride (0.22 g, 1.50 mmol), N-ethyldiisopropylamine (0.52 mL, 3.00 mmol) and methyl-phenyl-carbamic acid 5-(4-chloromethyl-benzoylamino)-pyridin-2-yl ester (0.40 g, 1.00 mmol) and stirring for 3 days yielded the title compound (341 mg, 72% yield) as a white solid.

¹H NMR (300 MHz, CDCl₃): δ=0.92 (s, 6H), 1.40 (t, 4H), 2.40 (m, 4H), 3.40 (br.s, 3H), 3.54 (s, 2H), 6.94 (br.s, 1H), 7.27 (m, 1H), 7.38 (m, 6H), 7.83 (d, 2H), 8.14 (dd, 1H), 8.37 (d, 1H), 8.54 (br.s, 1H); HPLC-MS (Method A): m/z=473 (M+H)⁺; Rt=2.91 min.

### Example 50

### [cis-Methyl-phenyl-carbamic acid 5-[4-(2,6-dimethyl-piperidin-1-ylmethyl)-benzoylamino]-pyridin-2-yl ester]

Using the procedure as described in Example 46 and starting from cis-2,6-dimethyl-piperidine (226 mg, 2.00 mmol) and methyl-phenyl-carbamic acid 5-(4-chloromethyl-benzoylamino)-pyridin-2-yl ester (0.40 g, 1.00 mmol) and stirring for 3 days yielded the title compound (410 mg, 87% yield).

¹H NMR (300 MHz, CDCl₃): δ=1.00 (d, 6H), 1.32 (m, 3H), 1.51-1.70 (m, 3H), 2.48 (m, 2H), 3.40 (br.s, 3H), 3.80 (s, 2H), 6.92 (br.s, 1H), 7.27 (m, 1H), 7.36 (m, 4H), 7.46 (d, 2H), 7.83 (d, 2H), 8.11 (dd, 1H), 8.40 (d, 1H), 8.78 (br.s, 1H); HPLC-MS (Method A): m/z=473 (M+H)⁺; Rt=2.77 min.

### Example 51

### [Methyl-phenyl-carbamic acid 5-[4-(4-oxo-piperidin-1-ylmethyl)-benzoylamino]-pyridin-2-yl ester]

Using the procedure as described in Example 46 and starting from 4-ketopiperidine hydrate hydrochloride (154 mg, 1.00 mmol), N-ethyldiisopropylamine (259 mg, 2.00 mmol) and methyl-phenyl-carbamic acid 5-(4-chloromethyl-benzoylamino)-pyridin-2-yl ester (0.40 g, 1.00 mmol) and stirring for 2 days yielded the title compound (358 mg, 78% yield).

¹H NMR (300 MHz, CDCl₃): δ=2.47 (m, 4H), 2.74 (m, 4H), 3.42 (br.s, 3H), 3.67 (s, 2H), 6.93 (br.s, 1H), 7.26 (m, 1H), 7.38 (m, 4H), 7.42 (d, 2H), 7.87 (d, 2H), 8.12 (dd, 1H), 8.39 (d, 1H), 8.66 (br.s, 1H); HPLC-MS (Method A): m/z=459 (M+H)⁺; Rt=2.31 min.

### Example 52

### [cis-Methyl-phenyl-carbamic acid 5-[4-(2,6-dimethyl-morpholin-4-ylmethyl)-benzoylamino]-pyridin-2-yl ester]

Using the procedure as described in Example 46 and starting from cis-2,6-dimethyl-morpholine (230 mg, 2.00 mmol) and methyl-phenyl-carbamic acid 5-(4-chloromethyl-benzoylamino)-pyridin-2-yl ester (0.40 g, 1.00 mmol) yielded the title compound (300 mg, 63% yield) as a white solid.

¹H NMR (300 MHz, CDCl₃): δ=1.12 (d, 6H), 1.77 (t, 2H), 2.66 (d, 2H), 3.38 (br.s, 3H), 3.50 (s, 2H), 3.70 (m, 2H), 6.89 (br.s, 1H), 7.25 (m, 1H), 7.36 (m, 6H), 7.86 (d, 2H), 8.06 (dd, 1H), 8.40 (d, 1H), 9.03 (s, 1H); HPLC-MS (Method A): m/z=475 (M+H)⁺; Rt=2.63 min.

### Example 53

### [Methyl-phenyl-carbamic acid 5-(4-thiomorpholin-4-ylmethyl-benzoylamino)-pyridin-2-yl ester]

Thiomorpholine (206 mg, 2.00 mmol) was added to a stirred solution of methyl-phenyl-carbamic acid 4-(4-chloromethyl-benzoylamino)-phenyl ester (396 mg, 1.00 mmol) and a catalytic amount of sodium iodide in dimethylformamide (5 mL). After stirring for 3 hours at room temperature, water was added to the reaction mixture and the precipitate was isolated by suction, washed thoroughly with water and dried in a vacuum oven at 40° C., yielding the title compound (424 mg, 92% yield) as an off-white solid.

¹H NMR (300 MHz, CDCl₃): δ=2.69 (m, 8H), 3.42 (br.s, 3H), 3.56 (s, 2H), 6.94 (br.s, 1H), 7.27 (m, 1H), 7.38 (m, 6H), 7.85 (d, 2H), 8.12 (dd, 1H), 8.37 (d, 1H), 8.50 (s, 1H); HPLC-MS (Method A): m/z=463 (M+H)⁺; Rt=2.61 min.

### Example 54

### [Methyl-phenyl-carbamic acid 5-[3-(2-hydroxy-1,1-dimethyl-ethyl)-thioureido]-pyridin-2-yl ester]

Methyl-phenyl-carbamic acid 5-amino-pyridin-2-yl ester (0.73 g, 3.00 mmol) was added to a solution of di-2-pyridyl thionocarbonate (0.70 g, 3.00 mmol) in dichloromethane (15 mL). After stirring for 2 hours 2-amino-2-methyl-1-propanol (0.27 g, 3.00 mmol), dissolved in a small amount of dichloromethane, was added in one portion. Stirring was continued overnight at room temperature. The product was purified by flash column chromatography (SiO₂, gradient of 80-100% ethyl acetate in heptane). Crystallisation from ethyl acetate/heptane yielded the title compound (907 mg, 81% yield) as a white solid.

¹H NMR (300 MHz, CDCl₃): δ=1.30 (s, 6H), 3.42 (br.s, 3H), 3.55 (s, 2H), 5.20 (br.s, 1H), 6.41 (s, 1H), 7.00 (br.s, 1H), 7.27 (m, 1H), 7.38 (m, 4H), 8.13 (s, 1H), 8.23 (br.s, 1H), 10.70 (br.s, 1H); HPLC-MS (Method A): m/z=375 (M+H)⁺; Rt=3.04 min.

### Example 55

### [Methyl-phenyl-carbamic acid 5-[3-(1-methyl-cyclopropyl)-thioureido]-pyridin-2-yl ester]

Methyl-phenyl-carbamic acid 5-amino-pyridin-2-yl (243 mg, 1.00 mmol) was added to a solution of di-2-pyridyl thionocarbonate (232 mg, 1.00 mmol) in dichloromethane (5 mL). The reaction mixture was stirred at room temperature for 2 hours. 1-Methylcyclopropylamine hydrochloride (113 mg, 1.05 mmol) was added in one portion, followed by triethylamine (146 μL, 1.05 mmol). Stirring was continued overnight at room temperature. Purification by flash column chromatography (SiO₂, ethyl acetate/heptane 60/40) yielded the title compound (318 mg, 89% yield) as a white solid.

¹H NMR (300 MHz, CDCl₃): δ=0.55-1.09 (3×br.s, 4H), 1.43 (s, 3H), 3.44 (br.s, 3H), 7.04 (br.s, 2H), 7.28 (m, 1H), 7.39 (m, 4H), 7.73-8.38 (3×br.s, 3H); HPLC-MS (Method A): m/z=357 (M+H)⁺; Rt=3.11 min.

### Example 56

### [Methyl-phenyl-carbamic acid 5-[3-(1-methyl-cyclobutyl)-thioureido]-pyridin-2-yl ester]

Using the procedure as described in Example 55 and starting from methyl-phenyl-carbamic acid 5-amino-pyridin-2-yl (243 mg, 1.00 mmol) and 1-methylcyclobutylamine hydrochloride (128 mg, 1.05 mmol) yielded the title compound (261 mg, 71% yield) as a white solid.

¹H NMR (300 MHz, CDCl₃): δ=1.59 (s, 3H), 1.81 (m, 2H), 2.02 (m, 2H), 2.16 (m, 2H), 3.32-3.60 (2×br.s, 3H), 6.63-7.07 (4×br.s, 2H), 7.28 (m, 1H), 7.38 (m, 4H), 7.69-8.00 (2×br.s, 3H); HPLC-MS (Method A): m/z=371 (M+H)⁺; Rt=3.68 min.

### Example 57

### [Methyl-phenyl-carbamic acid 5-(4-imidazol-1-yl-benzoylamino)-pyridin-2-yl ester]

At 40° C. 1-hydroxybenzotriazole hydrate (0.75 g, 5.55 mmol) and 1-(3-dimethylamino-propyl)-3-ethylcarbodiimide hydrochloride (1.06 g, 5.55 mmol) were added to a stirred solution of 4-imidazol-1-yl-benzoic acid (0.94 g, 5.00 mmol) in N,N-dimethylformamide (10 mL). After 30 minutes methyl-phenyl-carbamic acid 5-amino-pyridin-2-yl ester (1.35 g, 5.55 mmol) and N-ethyldiisopropylamine (0.97 mL, 5.55 mmol) were added and stirring was continued overnight at 40° C. The solvent was evaporated in vacuo, the crude product dissolved in 1M aqueous HCl (100 mL) and extracted with ethyl acetate (2×100 mL). Sodium carbonate was slowly added to the water layer. The solids were isolated by suction and dried overnight in a vacuum oven yielding the title compound (1.00 g, 50% yield) as an off-white solid.

¹H NMR (400 MHz, DMSO-d₆): δ=7.17 (s, 1H), 7.25 (d, 1H), 7.30 (m, 1H), 7.46 (m, 4H), 7.89 (d, 2H), 7.91 (s, 1H), 8.13 (d, 2H), 8.27 (dd, 1H), 8.44 (s, 1H), 8.69 (d, 1H), 10.58 (br.s, 1H); HPLC-MS (Method A): m/z=414 (M+H)⁺; Rt=2.40 min.

### Example 58

### [Methyl-phenyl-carbamic acid 5-(4-diethylamino-benzoylamino)-pyridin-2-yl ester]

At 40° C. 1-hydroxybenzotriazole hydrate (0.75 g, 5.55 mmol) and 1-(3-dimethylamino-propyl)-3-ethylcarbodiimide hydrochloride (1.06 g, 5.55 mmol) were added to a stirred solution of 4-diethylamine-benzoic acid (0.97 g, 5.00 mmol) in N,N-dimethylformamide (10 mL). After 30 minutes methyl-phenyl-carbamic acid 5-amino-pyridin-2-yl ester (1.35 g, 5.55 mmol) and N-ethyldiisopropylamine (0.97 mL, 5.55 mmol) were added and stirring is continued overnight at 40° C. The solvent was evaporated in vacuo. A small portion of the crude product (around 100 mg) was purified by preparative HPLC yielding the title compound (5 mg, 1% yield) as an off-white solid.

¹H NMR (400 MHz, DMSO-d₆): δ=1.12 (t, 6H), 3.42 (q, 4H), 6.73 (d, 2H), 7.18 (d, 1H), 7.29 (m, 1H), 7.45 (m, 4H), 7.87 (d, 2H), 8.24 (dd, 1H), 8.65 (d, 1H), 10.08 (s, 1H); HPLC-MS (Method A): m/z=419 (M+H)⁺; Rt=3.20 min.

### Example 59

### [Methyl-phenyl-carbamic acid 5-(4-[1,2,4]triazol-1-yl-benzoylamino)-pyridin-2-yl ester]

At 40° C. 1-hydroxybenzotriazole hydrate (0.13 g, 0.99 mmol) and 1-(3-dimethylamino-propyl)-3-ethylcarbodiimide hydrochloride (0.19 g, 0.99 mmol) were added to a stirred solution of 4-(1H-1,2,4 triazol-1yl)benzoic acid (0.16 g, 0.82 mmol) in N,N-dimethylformamide (5 mL). After 30 minutes methyl-phenyl-carbamic acid 5-amino-pyridin-2-yl ester (0.20 g, 0.82 mmol) and N-ethyldiisopropylamine (0.17 mL, 0.99 mmol) were added and stirring was continued overnight at 40° C. The solvent was evaporated in vacuo. A small portion of the crude product (around 100 mg) was purified by preparative HPLC yielding the title compound (10 mg, 1% yield) as a white solid.

¹H NMR (400 MHz, DMSO-d₆): δ=7.25 (d, 1H), 7.30 (m, 1H), 7.45 (m, 4H), 8.07 (d, 2H), 8.18 (d, 2H), 8.28 (dd, 1H), 8.32 (s, 1H), 8.69 (d, 1H), 9.45 (s, 1H), 10.62 (s, 1H); HPLC-MS (Method A): m/z=415 (M+H)⁺; Rt=3.13 min.

### Example 60

### [Methyl-phenyl-carbamic acid 5-(3,3-dipropyl-thioureido)-pyridin-2-yl ester]

Methyl-phenyl-carbamic acid 5-amino-pyridin-2-yl ester (2.92, 12.0 mmol), dissolved in N,N-dimethylformamide (20 mL), was added dropwise to a stirred solution of di-2-pyridyl thionocarbonate (2.79 g, 12.0 mmol) in N,N-dimethylformamide (15 mL). After stirring for 3 hours at room temperature, one-fourth of the solution (3.00 mmol) was added to di-n-propylamine (334 mg, 3.30 mmol), followed by the addition of triethylamine (0.46 mL). The reaction mixture was stirred at room temperature overnight. Purification by flash column chromatography (SiO₂, ethyl acetate/heptane 50/50) yielded the title compound (252 mg, 22% yield) as a white solid.

¹H NMR (300 MHz, CDCl₃): δ=0.94 (t, 6H), 1.72 (sextet, 4H), 3.42 (br.s, 3H), 3.60 (t, 4H), 6.86 (br.s, 1H), 7.26 (m, 1H), 7.38 (m, 4H), 7.49 (br.s, 1H), 7.72 (d, 1H), 8.10 (d, 1H); HPLC-MS (Method A): m/z=387 (M+H)⁺; Rt=4.07 min.

### Example 61

### [Methyl-phenyl-carbamic acid 5-(3,3-dibutyl-thioureido)-pyridin-2-yl ester]

Using the procedure as described in Example 60 and starting from methyl-phenyl-carbamic acid 5-amino-pyridin-2-yl ester (0.73 g, 3.00 mmol) and di-n-butylamine (427 mg, 3.30 mmol) yielded the title compound (283 mg, 23% yield) as a white solid.

¹H NMR (300 MHz, CDCl₃): δ=0.97 (t, 6H), 1.38 (sextet, 4H), 1.68 (m, 4H), 3.43 (br.s, 3H), 3.66 (t, 4H), 6.93 (br.s, 1H), 7.25 (m, 1H), 7.38 (m, 4H), 7.82 (d, 1H), 8.10 (d, 1H); HPLC-MS (Method A): m/z=415 (M+H)⁺; Rt=4.64 min.

### Example 62

### [Methyl-phenyl-carbamic acid 5-[(piperidine-1-carbothioyl)-amino]-pyridin-2-yl ester]

Using the procedure as described in Example 60 and starting from methyl-phenyl-carbamic acid 5-amino-pyridin-2-yl ester (0.73 g 3.00 mmol) and piperidine (281 mg, 3.30 mmol), followed by flash column chromatography (SiO₂, ethyl acetate/heptane 70/30) yielded the title compound (867 mg, 78% yield) as a white solid.

¹H NMR (400 MHz, CDCl₃): δ=1.63 (m, 6H), 3.41 (br.s, 1H), 3.82 (br.s, 4H), 6.83+6.91 (2×br.s, 1H), 7.27 (m, 1H), 7.37 (m, 4H), 7.60 (br.s, 1H), 7.68 (br.s, 1H), 8.08 (br.s, 1H); HPLC-MS (Method A): m/z=371 (M+H)⁺; Rt=3.51 min.

### Example 63

### [Methyl-phenyl-carbamic acid 5-[(4-methyl-piperidine-1-carbothioyl)-amino]-pyridin-2-yl ester]

Using the procedure as described in Example 60 and starting from methyl-phenyl-carbamic acid 5-amino-pyridin-2-yl ester (0.73 g 3.00 mmol) and 4-methylpiperidine (281 mg, 3.30 mmol), followed by flash column chromatography (SiO₂, ethyl acetate/heptane 60/40) yielded the title compound (0.94 g, 81% yield) as a white solid.

¹H NMR (400 MHz, CDCl₃): δ=0.95 (d, 3H), 1.22 (m, 2H), 1.68 (m, 3H), 2.99 (t, 2H), 3.41 (br.s, 3H), 4.67 (d, 2H), 6.84 (br.s, 1H), 7.28 (m, 1H), 7.37 (m, 4H), 7.52-7.87 (3×br.s, 2H), 8.19 (s, 1H); HPLC-MS (Method A): m/z=385 (M+H)⁺; Rt=3.85 min.

### Example 64

### [Methyl-phenyl-carbamic acid 5-[(4,4-dimethyl-piperidine-1-carbothioyl)-amino]-pyridin-2-yl ester]

Using the procedure as described in Example 60 and starting from methyl-phenyl-carbamic acid 5-amino-pyridin-2-yl ester (0.73 g 3.00 mmol), 4,4-dimethylpiperidine hydrochloride (494 mg, 3.30 mmol) and triethylamine (0.46 mL, 3.30 mmol) yielded the title compound (678 mg, 57% yield) as a white solid.

¹H NMR (400 MHz, CDCl₃): δ=1.00 (s, 6H), 1.43 (m, 4H), 3.42 (br.s, 3H), 3.83 (m, 4H), 6.87 (br.s, 1H), 7.27 (m, 1H), 7.38 (m, 4H), 7.53 (br.s, 1H), 7.62 (d, 1H), 8.10 (s, 1H); HPLC-MS (Method A): m/z=399 (M+H)⁺; Rt=4.06 min.

### Example 65

### [(4-Bromo-phenyl)-methyl-carbamic acid 4,4-dimethyl-2,6-dioxo-3,4,5,6-tetrahydro-2H-[1,3′]bipyridinyl-6′-yl ester]

A solution of N-(4-bromophenyl)-N-methylcarbamoyl chloride (0.50 g, 2.00 mmol) in dichloromethane (5 mL) was added to a stirred solution of 6′-hydroxy-4,4-dimethyl-4,5-dihydro-3H-[1,3′]bipyridinyl-2,6-dione (0.47 g, 2.00 mmol) and 1,4-diazabicyclo[2.2.2]octane (0.22 g, 2.00 mmol) in N,N-dimethylformamide (10 mL). After stirring for 4 hours at room temperature the solvent was evaporated in vacuo and the residue was purified by flash column chromatography (SiO₂, ethyl acetate/heptane 60/40) yielding the title compound (176 mg, 20% yield) as a white solid.

¹H NMR (300 MHz, CDCl₃): δ 1=1.20 (s, 6H), 2.68 (s, 4H), 3.43 (br.s, 3H), 7.16 (br.s, 1H), 7.27 (d, 2H), 7.50 (m, 3H), 8.09 (d, 1H); HPLC-MS (Method A): m/z=446+448 (M+H)⁺; Rt=3.88 min.

### Example 66

### [(4-Chloro-phenyl)-methyl-carbamic acid 4,4-dimethyl-2,6-dioxo-3,4,5,6-tetrahydro-2H-[1,3′]bipyridinyl-6′-yl ester]

Phosgene (20% in toluene, 45 mL) was added slowly by means of a syringe to a stirred solution of 6′-hydroxy-4,4-dimethyl-4,5-dihydro-3H-[1,3′]bipyridinyl-2,6-dione (2.11 g, 9.00 mmol) and N-ethyldiisopropylamine (1.62 mL, 9.30 mmol) in dichloromethane (90 mL). After stirring for 1.5 hours at room temperature the solvent was removed by evaporation in vacuo and the residue was redissolved in dichloromethane (45 mL). One ninth of this solution was added to a mixture of (4-chlorophenyl)methylamine (156 mg, 1.10 mmol) and 1,4-diazabicyclo[2.2.2]octane (112 mg, 1.00 mmol) in dichloromethane (2 mL). After standing overnight the solution was extracted twice with water. The dichloromethane layer was evaporated and the residue was purified by preparative HPLC yielding the title compound (103 mg, 26% yield) as a white solid.

¹H NMR (400 MHz, CDCl₃): δ=1.20 (s, 6H), 2.69 (s, 4H), 3.41 (br.s, 3H), 7.14 (br.s, 1H), 7.33 (m, 4H), 7.50 (br.d, 1H), 8.09 (br.s, 1H); HPLC-MS (Method A): m/z=402 (M+H)⁺; Rt=3.82 min.

### Example 67

### [(3,4-Dichloro-phenyl)-methyl-carbamic acid 4,4-dimethyl-2,6-dioxo-3,4,5,6-tetrahydro-2H-[1,3′]bipyridinyl-6′-yl ester]

Using the procedure as described in Example 66 and starting from (3,4-dichlorophenyl)-methylamine (194 mg, 1.10 mmol) yielded the title compound (53 mg, 12% yield) as a white solid.

¹H NMR (400 MHz, CDCl₃): δ=1.20 (s, 6H), 2.69 (s, 4H), 3.43 (br.s, 3H), 7.19 (br.s, 1H), 7.27 (m, 1H), 7.45 (d, 1H), 7.51 (m, 1H), 8.10 (br.s, 1H); HPLC-MS (Method A): m/z=436 (M+H)⁺; Rt=4.16 min.

### Example 68

### [(3-Chloro-phenyl)-methyl-carbamic acid 4,4-dimethyl-2,6-dioxo-3,4,5,6-tetrahydro-2H-[1,3′]bipyridinyl-6′-yl ester]

Using the procedure as described in Example 66 and starting from (3-chlorophenyl)-methylamine (156 mg, 1.10 mmol) yielded the title compound (107 mg, 27% yield) as a white solid.

¹H NMR (400 MHz, CDCl₃): δ=1.20 (s, 6H), 2.69 (s, 4H), 3.44 (br.s, 3H), 7.19 (br.s, 1H), 7.29 (m, 3H), 7.40 (s, 1H), 7.50 (d, 1H), 8.10 (s, 1H); HPLC-MS (Method A): m/z=402 (M+H)⁺; Rt=3.82 min.

### Example 69

### [Methyl-p-tolyl-carbamic acid 4,4-dimethyl-2,6-dioxo-3,4,5,6-tetrahydro-2H-[1,3′]bipyridinyl-6′-yl ester]

Using the procedure as described in Example 66 and starting from (4-methylphenyl)-methylamine (133 mg, 1.10 mmol) yielded the title compound (118 mg, 31% yield) as a white solid.

¹H NMR (400 MHz, CDCl₃): δ=1.20 (s, 6H), 2.34 (s, 3H), 2.68 (s, 4H), 3.39 (br.s, 3H), 7.11 (br.s, 1H), 7.18 (d, 2H), 7.25 (d, 2H), 7.46 (br.d, 1H), 8.08 (br.s, 1H); HPLC-MS (Method A): m/z=382 (M+H)⁺; Rt=3.71 min.

### Example 70

### [(3-Fluoro-phenyl)-methyl-carbamic acid 4,4-dimethyl-2,6-dioxo-3,4,5,6-tetrahydro-2H-[1,3′]bipyridinyl-6′-yl ester]

Using the procedure as described in Example 66 and starting from (3-fluorophenyl)-methylamine (138 mg, 1.10 mmol) yielded the title compound (60 mg, 16% yield) as a white solid.

¹H NMR (400 MHz, CDCl₃): δ=1.20 (s, 6H), 2.69 (s, 4H), 3.45 (br.s, 3H), 6.98 (dt, 1H), 7.12 (br.d, 1H), 7.19 (d, 1H), 7.34 (m, 1H), 7.50 (d, 1H), 8.10 (s, 1H); HPLC-MS (Method A): m/z=386 (M+H)⁺; Rt=3.56 min.

### Example 71

### [Methyl-m-tolyl-carbamic acid 4,4-dimethyl-2,6-dioxo-3,4,5,6-tetrahydro-2H-[1,3′]bipyridinyl-6′-yl ester]

Using the procedure as described in Example 66 and starting from (3-methylphenyl)-methylamine (133 mg, 1.10 mmol) yielded the title compound (100 mg, 26% yield) as a white solid.

¹H NMR (400 MHz, CDCl₃): δ=1.20 (s, 6H), 2.37 (s, 3H), 2.68 (s, 4H), 3.40 (br.s, 3H), 7.07 (d, 1H), 7.15 (m, 3H), 7.27 (t, 1H), 7.48 (br.d, 1H), 8.09 (br.s, 1H); HPLC-MS (Method A): m/z=382 (M+H)⁺; Rt=3.72 min.

### Example 72

### [(4-Methoxy-phenyl)-methyl-carbamic acid 4,4-dimethyl-2,6-dioxo-3,4,5,6-tetrahydro-2H-[1,3′]bipyridinyl-6′-yl ester]

By means of syringe phosgene (20% in toluene, 5 mL) was slowly added to a solution of 6′-hydroxy-4,4-dimethyl-4,5-dihydro-3H-[1,3′]bipyridinyl-2,6-dione (333 mg, 1.42 mmol) and N-ethyldiisopropylamine (0.14 g, 1.10 mmol) in dichloromethane (15 mL). After stirring for 0.5 hours at room temperature the solvent and excess of phosgene were removed by evaporation in vacuo. The residue was redissolved in dichloromethane. The solution was slowly added to a cold solution of (4-methoxyphenyl)methylamine (151 mg, 1.10 mmol) and 1,4-diazabicyclo[2.2.2]octane (0.11 mmol, 1.00 mmol) in dichloromethane (4 mL). After stirring overnight the solution was extracted twice with water and evaporated in vacuo. The residue was purified by preparative HPLC. Crystallisation from ethyl acetate yielded the title compound (10 mg, 3% yield) as a white solid.

¹H NMR (400 MHz, CDCl₃): δ=1.21 (s, 6H), 2.68 (s, 4H), 3.37+3.49 (2×br.s, 3H), 3.81 (s, 3H), 6.90 (d, 2H), 7.10 (br.d, 1H), 7.26 (m, 3H), 7.45 (br.d, 1H), 8.06 (br.s, 1H); HPLC-MS (Method A): m/z=398 (M+H)⁺; Rt=3.20 min.

### Example 73

### [(3-Methoxy-phenyl)-methyl-carbamic acid 4,4-dimethyl-2,6-dioxo-3,4,5,6-tetrahydro-2H-[1,3′]bipyridinyl-6′-yl ester]

Using the procedure as described in Example 72 and starting from 6′-hydroxy-4,4-dimethyl-4,5-dihydro-3H-[1,3′]bipyridinyl-2,6-dione (333 mg, 1.42 mmol) and (3-methoxyphenyl)-methylamine (151 mg, 1.10 mmol) yielded the title compound as a white solid.

¹H NMR (400 MHz, CDCl₃): δ=1.21 (s, 6H), 2.69 (s, 4H), 3.43 (br.s, 3H), 3.81 (s, 3H), 6.82 (d, 1H), 6.95 (m, 2H), 7.14 (br.s, 1H), 7.30 (d, 1H), 7.48 (br.d, 1H), 8.09 (br.s, 1H); HPLC-MS (Method A): m/z=398 (M+H)⁺; Rt=3.14 min.

### Example 74

### [Methyl-(3-trifluoromethyl-phenyl)-carbamic acid 4,4-dimethyl-2,6-dioxo-3,4,5,6-tetrahydro-2H-[1,3′]bipyridinyl-6′-yl ester]

Using the procedure as described in Example 72 and starting from 6′-hydroxy-4,4-dimethyl-4,5-dihydro-3H-[1,3′]bipyridinyl-2,6-dione (333 mg, 1.42 mmol) and (3-trifluoromethyl-phenyl)methylamine (193 mg, 1.10 mmol) yielded the title compound as a white solid.

¹H NMR (400 MHz, CDCl₃): δ=1.19 (s, 6H), 2.67 (s, 4H), 3.48 (br.s, 3H), 7.19 (br.s, 1H), 7.52 (m, 3H), 7.60 (s, 1H), 7.65 (s, 1H), 8.10 (s, 1H); HPLC-MS (Method A): m/z=436 (M+H)⁺; Rt=3.71 min.

### Example 75

### [(3-Bromo-phenyl)-methyl-carbamic acid 4,4-dimethyl-2,6-dioxo-3,4,5,6-tetrahydro-2H-[1,3′]bipyridinyl-6′-yl ester]

Using the procedure as described in Example 72 and starting from 6′-hydroxy-4,4-dimethyl-4,5-dihydro-3H-[1,3′]bipyridinyl-2,6-dione (333 mg, 1.42 mmol) and (3-bromophenyl)-methylamine (205 mg, 1.10 mmol) yielded the title compound as a white solid.

¹H NMR (400 MHz, CDCl₃): δ=1.21 (s, 6H), 2.68 (s, 4H), 3.44 (br.s, 3H), 7.05-7.60 (m, 6H), 8.10 (s, 1H); HPLC-MS (Method A): m/z=446+448 (M+H)⁺; Rt=3.61 min.

### Example 76

### [(4-Fluoro-phenyl)-methyl-carbamic acid 4,4-dimethyl-2,6-dioxo-3,4,5,6-tetrahydro-2H-[1,3′]bipyridinyl-6′-yl ester]

Using the procedure as described in Example 72 and starting from 6′-hydroxy-4,4-dimethyl-4,5-dihydro-3H-[1,3′]bipyridinyl-2,6-dione (333 mg, 1.42 mmol) and (4-fluorophenyl)-methylamine (138 mg, 1.10 mmol) yielded the title compound as a white solid.

¹H NMR (400 MHz, CDCl₃): δ=1.20 (s, 6H), 2.68 (s, 4H), 3.38+3.50 (2×br.s, 3H), 7.07 (m, 3H), 7.33 (m, 2H), 7.48 (br.s, 1H), 8.08 (s, 1H); HPLC-MS (Method A): m/z=386 (M+H)⁺; Rt=3.48 min.

### Example 77

### [[4-(2-Hydroxy-ethyl)-phenyl]-methyl-carbamic acid 4,4-dimethyl-2,6-dioxo-3,4,5,6-tetrahydro-2H-[1,3′]bipyridinyl-6′-yl ester]

Step A:

A solution of 4-aminophenethylalcohol (2.03 g, 14.8 mmol) in ethyl formate (15 mL) was heated at reflux overnight. After cooling to room temperature heptane was added to the reaction mixture. The solvent was decanted from the oil and the last traces of solvent were removed in vacuo. Dichloromethane was added to the residue and the precipitate was isolated by suction, yielding N-[4-(2-hydroxyethyl)-phenyl]-formamide (1.95 g, 80% yield), which was used without further purification in the next step

¹H NMR (400 MHz, CDCl₃): δ=2.04 (br.s, 1H), 2.83 (m, 1H), 3.84 (m, 2H), 7.02+7.46 (2×d, 2H), 7.18+7.22 (2×d, 2H), 7.62+8.22 (br.s+br.d, 1H), 8.27+8.60 (s+d, 1H); HPLC-MS (Method A): m/z=148 (M-OH)⁺; Rt=1.00 min.

Step B:

Under a nitrogen atmosphere, a solution of N-[4-(2-hydroxyethyl)-phenyl]-formamide (1.50 g, 9.10 mmol) in dry tetrahydrofuran (50 mL) was added dropwise to a stirred suspension of lithium aluminium hydride (0.76 g, 20.0 mmol) in dry tetrahydrofuran (50 mL). After refluxing for 1 hour, the solution was cooled to room temperature and 1M aqueous potassium hydroxide (1.9 mL) was slowly added. Celite was added and the solids are removed by filtration. Evaporation of the solvent in vacuo yielded 2-(4-methylamino-phenyl)-ethanol (1.00 g, yield: 73%), which was used in the next step without further purification.

¹H NMR (400 MHz, CDCl₃): δ=2.76 (t, 2H), 2.82 (s, 3H), 3.79 (t, 2H), 6.58 (d, 2H), 7.05 (d, 2H); HPLC-MS (Method A): m/z=152 (M+H)⁺; Rt=0.88 min.

Step C:

N-Ethyldiisopropylamine (0.7 mL, 4.00 mmol) and chlorotrimethylsilane (0.13 mL, 1.00 mmol) are added to a stirred solution of 2-(4-methylamino-phenyl)-ethanol (151 mg, 1.00 mmol) in dichloromethane (10 mL). In a second reaction flask N-ethyldiisopropylamine (0.21 mL, 1.20 mmol) and phosgene (20% in toluene, 5 mL) are added to a stirred solution of 6′-hydroxy-4,4-dimethyl-4,5-dihydro-3H-[1,3′]bipyridinyl-2,6-dione (234 mg, 1.00 mmol) in dichloromethane (5 mL). After 1.5 hours this solution is evaporated in vacuo. The residue is redissolved in dichloromethane (5 mL) and slowly added to the first solution. After stirring for 1 hour the solvent is evaporated in vacuo and the residue is purified by preparative HPLC yielding the title compound as a white solid.

¹H NMR (400 MHz, CDCl₃): δ=1.20 (s, 6H), 1.92 (br.s, 1H), 2.68 (s, 4H), 2.86 (t, 2H), 3.40 (br.s, 3H), 3.85 (t, 2H), 7.12 (br.s, 1H), 7.20-7.34 (m, 4H), 7.48 (br.s, 1H), 8.07 (s, 1H); HPLC-MS (Method A): m/z=412 (M+H)⁺; Rt=2.56 min.

Further examples are:
Methyl-phenyl-carbamic acid 5-(4-piperidin-1-yl-benzoylamino)-pyridin-2-yl ester
Methyl-phenyl-carbamic acid 5-[4-(2-methyl-piperidin-1-yl)-benzoylamino]-pyridin-2-yl ester
Methyl-phenyl-carbamic acid 5-[4-(3-methyl-piperidin-1-yl)-benzoylamino]-pyridin-2-yl ester
Methyl-phenyl-carbamic acid 5-[4-(4-methyl-piperidin-1-yl)-benzoylamino]-pyridin-2-yl ester
Methyl-phenyl-carbamic acid 4-[4-(2-ethyl-piperidin-1-yl)-benzoylamino]-pyridin-2-yl ester
Methyl-phenyl-carbamic acid 4-[4-(4,4-dimethyl-piperidin-1-yl)-benzoylamino]-pyridin-2-yl ester
Methyl-phenyl-carbamic acid 4-[4-(2,6-dimethyl-piperidin-1-yl)-benzoylamino]-pyridin-2-yl ester
Methyl-phenyl-carbamic acid 4-[4-(2,4,6-trimethyl-piperidin-1-yl)-benzoylamino]-pyridin-2-yl ester
Methyl-phenyl-carbamic acid 4-[4-(2,6-dimethyl-piperidin-1-ylmethyl)-benzoylamino]-pyridin-2-yl ester
Methyl-phenyl-carbamic acid 4-[4-(4,4-dimethyl-piperidin-1-ylmethyl)-benzoylamino]-pyridin-2-yl ester
Methyl-phenyl-carbamic acid 4-[4-(2,4,6-trimethyl-piperidin-1-ylmethyl)-benzoylamino]-pyridin-2-yl ester
Methyl-phenyl-carbamic acid 5-[4-(2-piperidin-1-yl-ethyl)-benzoylamino]-pyridin-2-yl ester
Methyl-phenyl-carbamic acid 5-{4-[2-(2-methyl-piperidin-1-yl)-ethyl]-benzoylamino}-pyridin-2-yl ester
Methyl-phenyl-carbamic acid 5-{4-[2-(3-methyl-piperidin-1-yl)-ethyl]-benzoylamino}-pyridin-2-yl ester
Methyl-phenyl-carbamic acid 5-{4-[2-(4-methyl-piperidin-1-yl)-ethyl]-benzoylamino}-pyridin-2-yl ester
Methyl-phenyl-carbamic acid 5-{4-[2-(2-ethyl-piperidin-1-yl)-ethyl]-benzoylamino}-pyridin-2-yl ester
Methyl-phenyl-carbamic acid 5-{4-[2-(4,4-dimethyl-piperidin-1-yl)-ethyl]-benzoylamino}-pyridin-2-yl ester
Methyl-phenyl-carbamic acid 5-{4-[2-(2,6-dimethyl-piperidin-1-yl)-ethyl]-benzoylamino}-pyridin-2-yl ester
Methyl-phenyl-carbamic acid 5-{4-[2-(2,4,6-trimethyl-piperidin-1-yl)-ethyl]-benzoylamino}-pyridin-2-yl ester
   Pharmacological Methods

Compounds of formula I may be evaluated in vitro for their efficacy and potency to inhibit HSL, and such evaluation may be performed as described below.

Assays

Hormone-Sensitive Lipase (HSL)

Materials. The Hormone-sensitive lipase was provided by Dr. Cecilia Holm, from Lund University Sweden or produced and purified by Novo Nordisk (NN) using the reagents and protocols used by Dr. Holm. The substrates used are: ³H-labeled triolein (TO) from Amersham, Buckinghamshire, U.K. cat No. TRA191; 5-20 Ci/mmol dissolved in toluene, triolein (Sigma, Cat. No. T-1740), fluorochrome-labeled triacylglyceride (cis-octadec-9-enoic acid 2-[12-(7-nitrobenzo[1,2,5]oxadiazol-4-ylamino)dodecanoyloxy]-1-cis-octadec-9-enoyloxymethyl-ethyl ester) prepared by Novo Nordisk (NN) by conventional methods, and 1,3-(di[³H]-stearin), 2-(PEG-Biotin)glycerol prepared in collaboration with Amersham Pharmacia Biotech, UK and described in WO 01/073442. Phosphatidyl choline (PC) and phosphatidyl inositol (PI) are from Sigma (St Luis Mo. cat. Nos. P-3556 and P-5954 respectively). All other reagents are of commercial grade and obtained from various commercial sources.

Methods.

3190.1: Assay for determination of percent inhibition of hormone sensitive lipase by compound at 10 μM sample concentration.

A lipid emulsion with fluorochrome-labeled triacylglyceride and phospholipid is used as substrate with a standard concentration of highly purified HSL (12 μg/mL initial concentration corresponding to 600 ng/mL final concentration). BSA is added as product acceptor. The transfer of the fluorochrome from the lipid phase to the water (BSA) phase changes the fluorescent properties of the fluorochrome. The changes can be monitored on a fluorimeter with an excitation wavelength of 450 nm and an emission wavelength of 545 nm.

Compound and HSL (20 μL compound, 10 μL enzyme and 70 μL PED-BSA buffer) is pre-incubated for 30 min at 25° C. before addition of substrate (100 μL). Amount of formed product is measured after 120 min incubation at 37° C.

Results are given as percent activity relative to a non-inhibited sample (no compound). 3190.2: Assay for determination of IC₅₀ value for the inhibition of hormone sensitive lipase by compound. Standard concentrations of compound are 100 μM and 5-fold dilutions (initial concentration corresponding to 10 μM final concentration and 5-fold).

A lipid emulsion with fluorochrome-labeled triacylglyceride and phospholipid is used as substrate with a standard concentration of highly purified HSL (12 μg/mL initial concentration corresponding to 600 ng/mL final concentration). BSA is added as product acceptor. The transfer of the fluorochrome from the lipid phase to the water (BSA) phase changes the fluorescent properties of the fluorochrome. The changes can be monitored on a fluorimeter with an excitation wavelength of 450 nm and an emission wavelength of 545 nm.

Compound and HSL (20 μL compound, 10 L enzyme and 70 μL PED-BSA buffer) is pre-incubated for 30 min at 25° C. before addition of substrate (100 μL). Amount of formed product is measured after 120 min incubation at 37° C.

Results are given as IC₅₀ values after 4PL fit of obtained activity data.

Results

With these methods the compounds of the examples are found to be inhibitors of HSL:

TABLE 1
Inhibition of HSL by compounds of the examples according to above
assay 3190.1 (% activity relative to non-inhibited sample).

| | | Test 3190.1 |
|---|---|---|
| Example | | HSL_FL |
| No. | Compound | % ACTIVITY |
| 1 | Methyl-phenyl-carbamic acid 5- | 0 |
| | [2-(4,4-dimethyl-2,6-dioxo- | |
| | piperidin-1-yl)-ethyl]-pyridin- | |
| | 2-yl ester | |
| 2 | [6-(Methyl-phenyl-carbamoyloxy)- | 1 |
| | pyridin-3-ylamino]-acetic | |
| | acid tert-butyl ester | |
| 5 | Methyl-phenyl-carbamic acid 5- | 9 |
| | (5-oxo-2-thioxo-imidazolidin- | |
| | 1-yl)-pyridin-2-yl ester | |
| 8 | Methyl-phenyl-carbamic acid 5- | 3 |
| | (4-ethyl-5-oxo-2-thioxo- | |
| | imidazolidin-1-yl)-pyridin-2- | |
| | yl ester | |
| 12 | Methyl-phenyl-carbamic acid 5- | 2 |
| | [3-(2,2-dimethyl-propyl)- | |
| | thioureido]-pyridin-2-yl ester | |
| 16 | Methyl-phenyl-carbamic acid 5- | 1 |
| | (3-butyl-thioureido)-pyridin- | |
| | 2-yl ester | |
| 21 | Methyl-phenyl-carbamic acid 5- | 1 |
| | (3-methoxy-benzoylamino)- | |
| | pyridin-2-yl ester | |
| 28 | Methyl-phenyl-carbamic acid 5- | 1 |
| | (2-fluoro-3-trifluoromethyl- | |
| | benzoylamino)-pyridin-2-yl ester | |
| 31 | Methyl-phenyl-carbamic acid | 3 |
| | 5-(3-dimethylamino- | |
| | benzoylamino)-pyridin-2-yl ester | |
| 32 | (2-Methoxy-phenyl)-methyl- | 6 |
| | carbamic acid 4,4-dimethyl-2,6- | |
| | dioxo-3,4,5,6-tetrahydro-2H- | |
| | [1,3′]bipyridinyl-6′-yl ester | |
| 35 | Methyl-phenyl-carbamic acid 5- | 8 |
| | (3-tert-butyl-ureido)-pyridin- | |
| | 2-yl ester | |
| 41 | Methyl-phenyl-carbamic acid 5- | 3 |
| | [4-(4-methyl-piperidin-1- | |
| | ylmethyl)-benzoylamino]- | |
| | pyridin-2-yl ester | |
| 43 | Methyl-phenyl-carbamic acid 5- | 7 |
| | [4-(2-methyl-piperidin-1- | |
| | ylmethyl)-benzoylamino]- | |
| | pyridin-2-yl ester | |
| 47 | Methyl-phenyl-carbamic acid 5- | 7 |
| | (4-pyrrolidin-1-ylmethyl- | |
| | benzoylamino)-pyridin-2-yl ester | |
| 52 | Methyl-phenyl-carbamic acid 5- | 4 |
| | [4-(2,6-dimethyl-morpholin- | |
| | 4-ylmethyl)-benzoylamino]- | |
| | pyridin-2-yl ester | |
| 57 | Methyl-phenyl-carbamic acid 5- | 17 |
| | (4-imidazol-1-yl- | |
| | benzoylamino)-pyridin-2-yl ester | |
| 58 | Methyl-phenyl-carbamic acid | 11 |
| | 5-(4-diethylamino- | |
| | benzoylamino)-pyridin-2-yl ester | |
| 65 | (4-Bromo-phenyl)-methyl-carbamic | 0 |
| | acid 4,4-dimethyl-2,6- | |
| | dioxo-3,4,5,6-tetrahydro-2H- | |
| | [1,3′]bipyridinyl-6′-yl ester | |
| 63 | Methyl-phenyl-carbamic acid 5- | 2 |
| | [(4-methyl-piperidine-1- | |
| | carbothioyl)-amino]-pyridin-2- | |
| | yl ester | |
| 72 | (4-Methoxy-phenyl)-methyl-carbamic | 2 |
| | acid 4,4-dimethyl-2,6- | |
| | dioxo-3,4,5,6-tetrahydro-2H- | |
| | [1,3′]bipyridinyl-6′-yl ester | |
| 77 | [4-(2-Hydroxy-ethyl)-phenyl]- | 12 |
| | methyl-carbamic acid 4,4- | |
| | dimethyl-2,6-dioxo-3,4,5,6- | |
| | tetrahydro-2H-[1,3′]bipyridinyl-6′- | |
| | yl ester | |

1. A compound of formula (I): 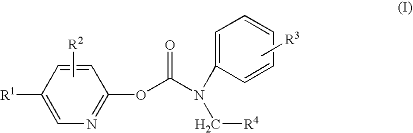
wherein
R¹ and R² are independently selected from hydrogen, hydroxy, sulfanyl, amino, amide, urea, thiourea, benzamide, thioamide, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, amide, urea, thiourea, benzamide, thioamide, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, thioxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R³, and R⁴ are independently selected from hydrogen, hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
with the proviso that said compound is not
2,2-Dimethyl-N-[6-(methyl-phenyl-carbamoyloxy)-pyridin-3-yl]-succinamic acid, 3,3-Dimethyl-4-[6-(methyl-phenyl-carbamoyloxy)-pyridin-3-ylcarbamoyl]-butyric acid, Methyl-phenyl-carbamic acid pyridin-2-yl ester, Methyl-phenyl-carbamic acid 5-chloro-pyridin-2-yl ester, Methyl-phenyl-carbamic acid 5-trifluoromethyl-pyridin-2-yl ester, Methyl-phenyl-carbamic acid 3-chloro-5-trifluoromethyl-pyridin-2-yl ester, Methyl-phenyl-carbamic acid 5-benzoylamino-pyridin-2-yl ester, Methyl-phenyl-carbamic acid 5-(cyclohexanecarbonyl-amino)-pyridin-2-yl ester, Methyl-phenyl-carbamic acid 4,4-dimethyl-2,6-dioxo-3,4,5,6-tetrahydro-2H-[1,3′]bipyridinyl-6′-yl ester, Methyl-phenyl-carbamic acid 5-(2,2-dimethyl-propionylamino)-pyridin-2-yl ester, Methyl-phenyl-carbamic acid 5-(2-cyclohexyl-acetylamino)-pyridin-2-yl ester, Methyl-phenyl-carbamic acid 5-(3,3-dimethyl-butyrylamino)-pyridin-2-yl ester, Methyl-phenyl-carbamic acid 5-[(pyridine-2-carbonyl)-amino]-pyridin-2-yl ester, Methyl-phenyl-carbamic acid 5-[(6-chloropyridine-3-carbonyl)-amino]-pyridin-2-yl ester, Methyl-phenyl-carbamic acid 5-(2,2-dimethyl-propylcarbamoyl)-pyridin-2-yl ester, Methyl-phenyl-carbamic acid 2,6-dioxo-3,4,5,6-tetrahydro-2H-[1,3′]bipyridinyl-6′-yl ester, Methyl-phenyl-carbamic acid 5-(2,5-dioxo-pyrrolidin-1-yl)-pyridin-2-yl ester, Methyl-phenyl-carbamic acid 5-(4-trifluoromethyl-benzoylamino)-pyridin-2-yl ester, Methyl-phenyl-carbamic acid 5-(4-chloro-benzoylamino)-pyridin-2-yl ester, Methyl-phenyl-carbamic acid 5-(4-methoxy-benzoylamino)-pyridin-2-yl ester, Methyl-phenyl-carbamic acid 4,4-dimethyl-3,4,5,6-tetrahydro-2H-[1,3′]bipyridinyl-6′-yl ester, Methyl-phenyl-carbamic acid 5-amino-pyridin-2-yl ester, Methyl-phenyl-carbamic acid 5-benzenesulfonylamino-pyridin-2-yl ester, Methyl-phenyl-carbamic acid 5-(3,3-dimethyl-2,5-dioxo-pyrrolidin-1-yl)-pyridin-2-yl ester, Methyl-phenyl-carbamic acid 5-[3,3-dimethyl-5-(4-methyl-piperazin-1-yl)-5-oxo-pentanoylamino]-pyridin-2-yl ester, Methyl-phenyl-carbamic acid 5-[3,3-dimethyl-4-(pyridin-3-ylcarbamoyl)-butyrylamino]-pyridin-2-yl ester, Methyl-phenyl-carbamic acid 5-(3,3-dimethyl-5-morpholin-4-yl-5-oxo-pentanoylamino)-pyridin-2-yl ester, Methyl-phenyl-carbamic acid 5-[4-(2-dimethylamino-ethylcarbamoyl)-3,3-dimethyl-butyrylamino]-pyridin-2-yl ester; N-Methyl-N-phenylcarbamic acid 5-nitro-3-trifluoromethylpyridin-2-yl ester,
N-Methyl-N-phenylcarbamic acid 3-nitropyridin-2-yl ester, and
N-Methyl-N-phenylcarbamic acid 5-nitropyridin-2-yl ester;
as well as diastereomers, enantiomers or tautomeric forms thereof, mixtures of these, pharmaceutically acceptable salts thereof, pharmaceutically acceptable solvates thereof, or polymorphs.

2. A compound according to claim 1, wherein R² is selected from hydrogen, hydroxy, amino, halogen, C₁₋₆-alkyl, C₁₋₆-alkoxy, C₂₋₆-alkenyl, aryl, heteroaryl, C₃₋₈-heterocyclyl, amide, urea and thiourea and C₃₋₁₀-cycloalkyl, wherein each of hydroxy, amino, C₁₋₆-alkyl, C₁₋₆-alkoxy, C₂₋₆-alkenyl, aryl, heteroaryl, C₃₋₈-heterocyclyl, amide, urea and thiourea and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl.

3. A compound according to claim 2, wherein R² is halogen or hydrogen.

4. A compound according to claim 1, wherein R⁴ is selected from hydrogen, halogen, C₁₋₆-alkyl, C₂₋₆-alkenyl, wherein each of C₁₋₆-alkyl, C₂₋₆-alkenyl is optionally substituted with one or more substituents independently selected from hydroxy, halogen, C₁₋₆-alkyl, C₂₋₆-alkenyl, aryl, heteroaryl, C₃₋₈-heterocyclyl and C₃₋₁₀-cycloalkyl.

5. A compound according to claim 4, wherein R⁴ is selected from hydrogen, halogen and C₁₋₆-alkyl.

6. A compound according to claim 5, wherein R⁴ is hydrogen.

7. A compound according to claim 5, wherein R⁴ is halogen.

8. A compound according to claim 1, wherein R³ is selected from hydrogen, hydroxy, halogen, C₁₋₆-alkyl, C₂₋₆-alkenyl, wherein each of hydroxy, C₁₋₆-alkyl, C₂₋₆-alkenyl is optionally substituted with one or more substituents independently selected from hydroxy, halogen, C₁₋₆-alkyl, C₂₋₆-alkenyl, aryl, heteroaryl, C₃₋₈-heterocyclyl and C₃₋₁₀-cycloalkyl.

9. A compound according to claim 8, wherein R³ is selected from halogen, C₁₋₆-alkyl, methoxy, perhalomethyl and perhalomethoxy.

10. A compound according to claim 9, wherein R³ is selected from halogen, methyl, ethyl, isopropyl, methoxy and perhalomethyl.

11. A compound according to claim 10, wherein R³ is selected from halogen, methyl, methoxy and perhalomethyl.

12. A compound according to claim 1, wherein R¹ and R² are independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, Cam-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

13. A compound according to claim 1, wherein
R² is H, and
R¹ is 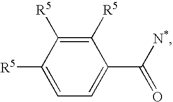
wherein
each R⁵ is independently selected from hydrogen, hydroxy, sulfanyl, amino, halogen, sulfo, C₁₋₆ alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester;
as well as diastereomers, enantiomers or tautomeric forms thereof, mixtures of these, pharmaceutically acceptable salts thereof, pharmaceutically acceptable solvates thereof, or polymorphs.

14. A compound according to claim 1, wherein
R² is H, and
R¹ is 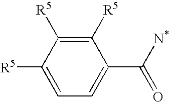
wherein,
each R⁵ is independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl; R³, and R⁴ are independently selected from hydrogen, hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester;
as well as diastereomers, enantiomers or tautomeric forms thereof, mixtures of these, pharmaceutically acceptable salts thereof, pharmaceutically acceptable solvates thereof, or polymorphs.

15. A compound according to claim 1, wherein
R² is H, and
R¹ is 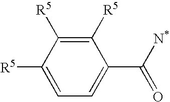
wherein,
each R⁵ is independently selected from hydrogen, hydroxy, sulfanyl, amino, halogen, sulfo, C₁-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R³, and R⁴ are hydrogen
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester;
as well as diastereomers, enantiomers or tautomeric forms thereof, mixtures of these, pharmaceutically acceptable salts thereof, pharmaceutically acceptable solvates thereof, or polymorphs.

16. A compound according to claim 1, wherein
R² is H, and
R¹ is 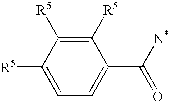
wherein,
each R⁵ is independently selected from hydrogen, hydroxy, sulfanyl, amino, halogen, sulfo, C₁₋₆-alkyl, C₁₋₆alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆alkyl, C₁₋₆alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆alkyl, C₁₋₆-alkoxy, C₂₋₆-alkenyl, aryl, heteroaryl, C₃₋₈-heterocyclyl, and C₃₋₁₀-cycloalkyl;
R³, and R⁴ are hydrogen
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester;
as well as diastereomers, enantiomers or tautomeric forms thereof, mixtures of these, pharmaceutically acceptable salts thereof, pharmaceutically acceptable solvates thereof, or polymorphs.

17. A compound according to claim 1, wherein
R² is H, and
R¹ is 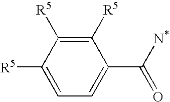
wherein,
each R⁵ is independently selected from hydrogen, hydroxy, amino, halogen, C₁₋₆-alkyl, C₁₋₆ alkoxy, C₂₋₆-alkenyl, aryl, heteroaryl, C₃₋₈-heterocyclyl and C₃₋₁₀-cycloalkyl, wherein each of hydroxy, C₁₋₆alkyl, C₁₋₆alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆alkyl, C₁₋₆alkoxy, C₂₋₆-alkenyl, aryl, heteroaryl, C₃₋₈-heterocyclyl, and C₃₋₁₀-cycloalkyl,
R³ and R⁴ are hydrogen
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester;
as well as diastereomers, enantiomers or tautomeric forms thereof, mixtures of these, pharmaceutically acceptable salts thereof, pharmaceutically acceptable solvates thereof, or polymorphs.

18. A compound according to claim 1, wherein
R² is H, and
R¹ is 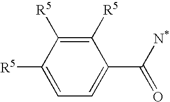
wherein,
each R⁵ is independently selected from hydrogen, hydroxy, amino, halogen, C₁₋₆alkyl, C₁₋₆ alkoxy, C₂₋₆-alkenyl, aryl, heteroaryl, C₃₋₈-heterocyclyl and C₃₋₁₀-cycloalkyl, wherein each of hydroxy, amino, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆alkyl, C₁₋₆alkoxy, C₂₋₆-alkenyl, aryl, heteroaryl, C₃₋₈-heterocyclyl, and C₃₋₁₀-cycloalkyl, R³ and R⁴ are hydrogen
with the proviso that said compound is not
Methyl-phenyl-carbamic acid 5-benzoylamino-pyridin-2-yl ester, Methyl-phenyl-carbamic acid 5-(4-trifluoromethyl-benzoylamino)-pyridin-2-yl ester, Methyl-phenyl-carbamic acid 5-(4-chlorobenzoylamino)-pyridin-2-yl ester, Methyl-phenyl-carbamic acid 5-(4-methoxy-benzoylamino)-pyridin-2-yl ester;
as well as diastereomers, enantiomers or tautomeric forms thereof, mixtures of these, pharmaceutically acceptable salts thereof, pharmaceutically acceptable solvates thereof, or polymorphs.

19. A compound according to claim 1, wherein R² is selected from from hydrogen, hydroxy, amino, halogen, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl.

20. A compound according to claim 1, wherein R³ is selected from fluor, chlor, methyl, methoxy, perhalomethyl or perhalomethoxy.

21. A compound according to claim 1, wherein R² is hydrogen.

22. A compound according to claim 1, wherein R² is selected from the group consisting of

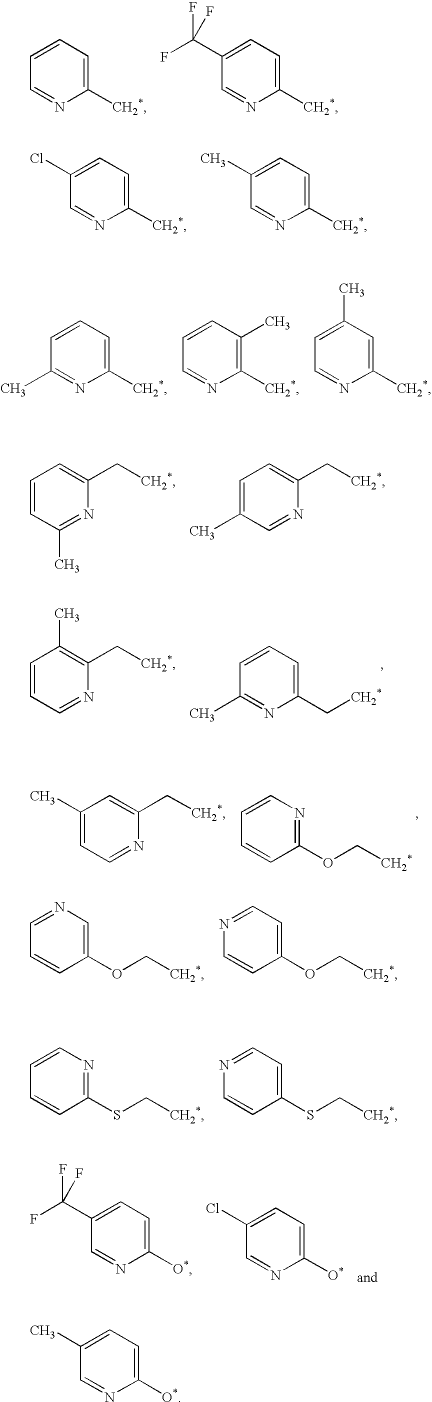

23. A compound according to claim 1, wherein R² is selected from the group consisting of 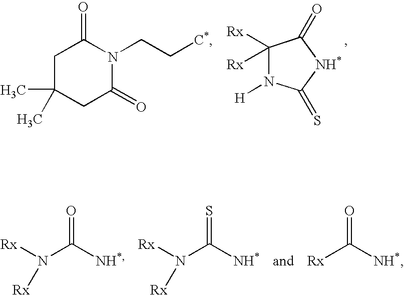
wherein each Rx is independently selected from hydroxy, sulfanyl, oxo, halogen, amino, sulfo, C₁₋₆-alkyl, C₁₋₆alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

24. A compound according to claim 1, wherein R² is selected from the group consisting of 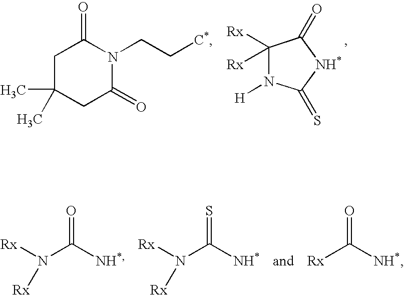
wherein each Rx is independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

25. A compound according to claim 1, wherein R² is selected from the group consisting of

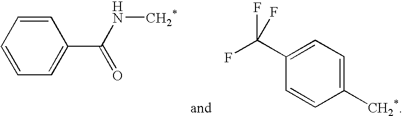

26. A compound according to claim 1, wherein R² is selected from the group consisting of

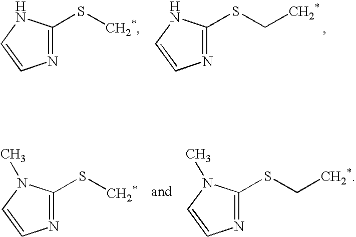

27. A compound according to claim 1, wherein R² is selected from the group consisting of

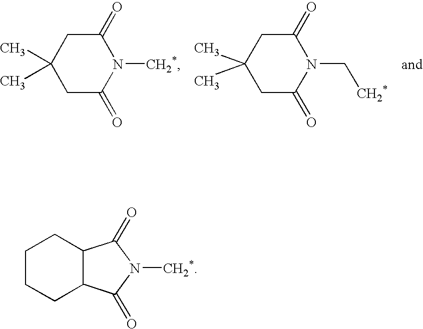

28. A compound according to claim 1, wherein R¹ is selected from the group consisting of

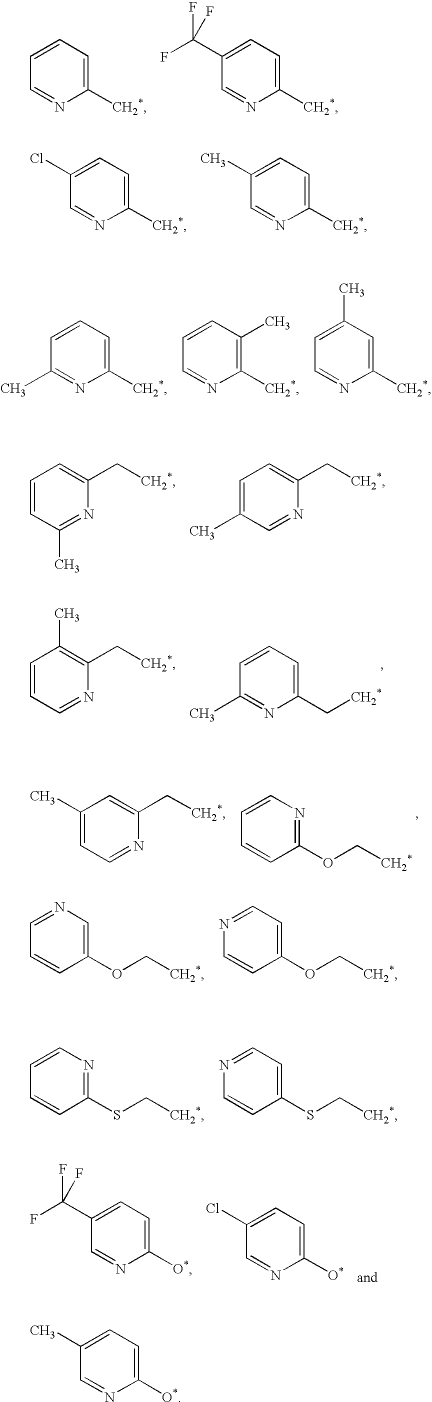

29. A compound according to claim 1, wherein R¹ is selected from the group consisting of

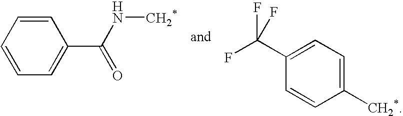

30. A compound according to claim 1, wherein R¹ is selected from the group consisting of

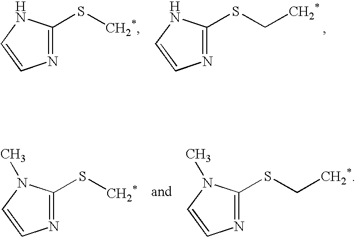

31. A compound according to claim 1, wherein R¹ is selected from the group consisting of

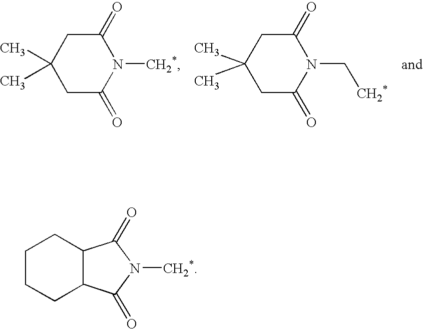

32. A compound according to claim 1, wherein R¹ is selected from the group consisting of 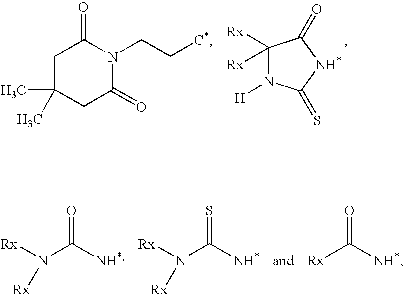
wherein each Rx is independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

33. A compound according to claim 1, wherein R¹ is selected from the group consisting of 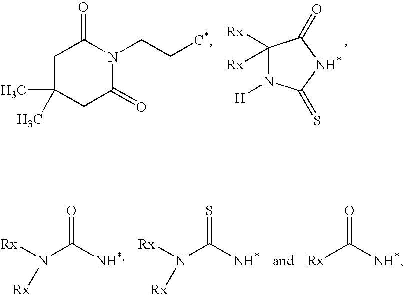
wherein each Rx is independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

34. A compound according to claim 1, wherein R¹ is selected from the group consisting of 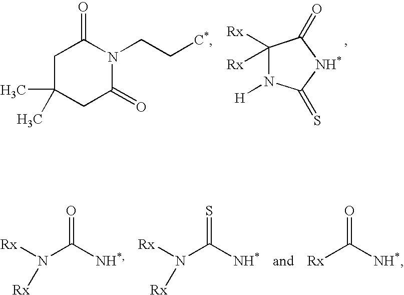
wherein each Rx is independently selected from halogen, perhalomethyl, perhalomethoxy, C₁₋₆ alkyl, heteroaryl, C₃₋₈-heterocyclyl, and C₃₋₁₀-cycloalkyl.

35. A compound according to claim 1, wherein R³ is hydrogen.

36. A compound according to claim 1, wherein R⁴ is hydrogen.

37. A compound according to claim 1, wherein R⁴ is selected from fluor, chlor, methyl, perhalomethyl or perhalomethoxy.

38. A compound according to claim 1, having one free —COOH group.

39. A compound according to claim 1, having one free amino group, or one monosubstituted amino group or one disubstituted amino group.

40. A compound according to claim 1, having one substituted or unsubstituted pyridine ring.

41. A compound according to claim 1, having one substituted or unsubstituted imidazole ring.

42. A compound according to claim 1, wherein the molar weight of said compound is less than 650 g/mole.

43. A compound according to claim 1, wherein the compound contains no ionisable group and wherein cLog P is in the range from 1.0 to 6.0.

44. A compound according to claim 1, wherein the compound contains no ionisable group and wherein cLog P is in the range from 1.0 to 5.0.

45. A compound according to claim 1, wherein the compound contains no ionisable group and wherein cLog P is in the range from 1.0 to 4.0.

46. A compound according to claim 1, wherein the compound contains no ionisable group and wherein cLog P is in the range from 2.0 to 4.0.

47. A compound according to claim 1, wherein the ACD LogD is in the range from 0.8 to 3.0.

48. A compound according to claim 1, wherein the number of H-bond donors is 0, 1, 2 or 3.

49. A compound according to claim 1, wherein the number of H-bond donors is 0, 1, or 2.

50. A compound according to claim 1, wherein the number of H-bond acceptors is in the range from 4 to 9.

51. A compound according to claim 1, wherein the number of H-bond acceptors is in the range from 6 to 8.

52. A compound according to claim 1, wherein the number of rotatable bonds of said compound is in the range from 4 to 14.

53. A compound according to claim 1, wherein the number of rotatable bonds of said compound is in the range from 8 to 12.

54. A compound according to claim 1, wherein the polar surface area (PSA) is in the range from 50 Å² to 120 Å².

55. A compound according to claim 1, wherein the polar surface area (PSA) is in the range from 60 Å² to 100 Å².

56. A compound according to claim 1, where the compound is selected from the group consisting of
[Methyl-phenyl-carbamic acid 5-[2-(4,4-dimethyl-2,6-dioxo-piperidin-1-yl)-ethyl]-pyridin-2-yl ester],
[[6-(Methyl-phenyl-carbamoyloxy)-pyridin-3-ylamino]-acetic acid tert-butyl ester],
[[6-(Methyl-phenyl-carbamoyloxy)-pyridin-3-ylamino]-acetic acid],
[Methyl-phenyl-carbamic acid 5-isothiocyanato-pyridin-2-yl ester],
[Methyl-phenyl-carbamic acid 5-(5-oxo-2-thioxo-imidazolidin-1-yl)-pyridin-2-yl ester],
[[6-(Methyl-phenyl-carbamoyloxy)-pyridin-3-ylamino]-acetic acid methyl ester],
[Methyl-phenyl-carbamic acid 5-(4,4-dimethyl-5-oxo-2-thioxo-imidazolidin-1-yl)-pyridin-2-yl ester],
[Methyl-phenyl-carbamic acid 5-(4-ethyl-5-oxo-2-thioxo-imidazolidin-1-yl)-pyridin-2-yl ester],
[(S)-Methyl-phenyl-carbamic acid 5-(4-isopropyl-5-oxo-2-thioxo-imidazolid in-1-yl)-pyridin-2-yl ester],
[Methyl-phenyl-carbamic acid 5-(4-tert-butoxymethyl-5-oxo-2-thioxo-imidazolidin-1-yl)-pyridin-2-yl ester],
[Methyl-phenyl-carbamic acid 5-(3-tert-butyl-thioureido)-pyridin-2-yl ester],
[Methyl-phenyl-carbamic acid 5-[3-(2,2-dimethyl-propyl)-thioureido]-pyridin-2-yl ester],
[Methyl-phenyl-carbamic acid 5-(3-isopropyl-thioureido)-pyridin-2-yl ester],
[Methyl-phenyl-carbamic acid 5-(3,3-diethyl-thioureido)-pyridin-2-yl ester],
[Methyl-phenyl-carbamic acid 5-(3-cyclohexyl-thioureido)-pyridin-2-yl ester],
[Methyl-phenyl-carbamic acid 5-(3-butyl-thioureido)-pyridin-2-yl ester],
[Methyl-phenyl-carbamic acid 5-(3-isobutyl-thioureido)-pyridin-2-yl ester],
[Methyl-phenyl-carbamic acid 5-(4-cyano-benzoylamino)-pyridin-2-yl ester],
[Methyl-phenyl-carbamic acid 5-(2-methyl-benzoylamino)-pyridin-2-yl ester],
[Methyl-phenyl-carbamic acid 5-(4-fluoro-benzoylamino)-pyridin-2-yl ester],
[Methyl-phenyl-carbamic acid 5-(3-methoxy-benzoylamino)-pyridin-2-yl ester],
[Methyl-phenyl-carbamic acid 5-(2-methoxy-benzoylamino)-pyridin-2-yl ester],
[Methyl-phenyl-carbamic acid 5-(3,4-dichloro-benzoylamino)-pyridin-2-yl ester],
[Methyl-phenyl-carbamic acid 5-(4-methyl-benzoylamino)-pyridin-2-yl ester],
[Methyl-phenyl-carbamic acid 5-(3-bromo-benzoylamino)-pyridin-2-yl ester],
[Methyl-phenyl-carbamic acid 5-(3-cyano-benzoylamino)-pyridin-2-yl ester],
[Methyl-phenyl-carbamic acid 5-(2-trifluoromethoxy-benzoylamino)-pyridin-2-yl ester],
[Methyl-phenyl-carbamic acid 5-(2-fluoro-3-trifluoromethyl-benzoylamino)-pyridin-2-yl ester],
[Methyl-phenyl-carbamic acid 5-(3-trifluoromethyl-benzoylamino)-pyridin-2-yl ester],
[Methyl-phenyl-carbamic acid 5-(3,4-difluoro-benzoylamino)-pyridin-2-yl ester],
[Methyl-phenyl-carbamic acid 5-(3-dimethylamino-benzoylamino)-pyridin-2-yl ester],
[Methyl-phenyl-carbamic acid 5-(3-dimethylamino-benzoylamino)-pyridin-2-yl ester],
[Methyl-phenyl-carbamic acid 5-(4-nitro-benzoylamino)-pyridin-2-yl ester],
[Methyl-phenyl-carbamic acid 5-(4-amino-benzoylamino)-pyridin-2-yl ester],
[Methyl-phenyl-carbamic acid 5-(3-tert-butyl-ureido)-pyridin-2-yl ester],
[Methyl-phenyl-carbamic acid 5-(4-formyl-benzoylamino)-pyridin-2-yl ester],
[Methyl-phenyl-carbamic acid 5-(4-morpholin-4-ylmethyl-benzoylamino)-pyridin-2-yl ester],
[Methyl-phenyl-carbamic acid 5-(4-hydroxymethyl-benzoylamino)-pyridin-2-yl ester],
[Methyl-phenyl-carbamic acid 5-(4-chloromethyl-benzoylamino)-pyridin-2-yl ester],
[Methyl-phenyl-carbamic acid 5-(4-piperidin-1-ylmethyl-benzoylamino)-pyridin-2-yl ester],
[Methyl-phenyl-carbamic acid 5-[4-(4-methyl-piperidin-1-ylmethyl)-benzoylamino]-pyridin-2-yl ester],
[Methyl-phenyl-carbamic acid 5-[4-(2-ethyl-piperidin-1-ylmethyl)-benzoylamino]-pyridin-2-yl ester],
[Methyl-phenyl-carbamic acid 5-[4-(2-methyl-piperidin-1-ylmethyl)-benzoylamino]-pyridin-2-yl ester],
[Methyl-phenyl-carbamic acid 5-[4-(3-methyl-piperidin-1-ylmethyl)-benzoylamino]-pyridin-2-yl ester],
Methyl-phenyl-carbamic acid 5-(4-piperidin-1-yl-benzoylamino)-pyridin-2-yl ester,
Methyl-phenyl-carbamic acid 5-[4-(2-methyl-piperidin-1-yl)-benzoylamino]-pyridin-2-yl ester,
Methyl-phenyl-carbamic acid 5-[4-(3-methyl-piperidin-1-yl)-benzoylamino]-pyridin-2-yl ester,
Methyl-phenyl-carbamic acid 5-[4-(4-methyl-piperidin-1-yl)-benzoylamino]-pyridin-2-yl ester,
Methyl-phenyl-carbamic acid 4-[4-(2-ethyl-piperidin-1-yl)-benzoylamino]-pyridin-2-yl ester,
Methyl-phenyl-carbamic acid 4-[4-(4,4-dimethyl-piperidin-1-yl)-benzoylamino]-pyridin-2-yl ester,
Methyl-phenyl-carbamic acid 4-[4-(2,6-dimethyl-piperidin-1-yl)-benzoylamino]-pyridin-2-yl ester,
Methyl-phenyl-carbamic acid 4-[4-(2,4,6-trimethyl-piperidin-1-yl)-benzoylamino]-pyridin-2-yl ester,
Methyl-phenyl-carbamic acid 4-[4-(2,6-dimethyl-piperidin-1-ylmethyl)-benzoylamino]-pyridin-2-yl ester,
Methyl-phenyl-carbamic acid 4-[4-(4,4-dimethyl-piperidin-1-ylmethyl)-benzoylamino]-pyridin-2-yl ester,
Methyl-phenyl-carbamic acid 4-[4-(2,4,6-trimethyl-piperidin-1-ylmethyl)-benzoylamino]-pyridin-2-yl ester,
Methyl-phenyl-carbamic acid 5-[4-(2-piperidin-1-yl-ethyl)-benzoylamino]-pyridin-2-yl ester,
Methyl-phenyl-carbamic acid 5-{4-[2-(2-methyl-piperidin-1-yl)-ethyl]-benzoylamino}-pyridin-2-yl ester,
Methyl-phenyl-carbamic acid 5-{4-[2-(3-methyl-piperidin-1-yl)-ethyl]-benzoylamino}-pyridin-2-yl ester,
Methyl-phenyl-carbamic acid 5-{4-[2-(4-methyl-piperidin-1-yl)-ethyl]-benzoylamino}-pyridin-2-yl ester,
Methyl-phenyl-carbamic acid 5-{4-[2-(2-ethyl-piperidin-1-yl)-ethyl]-benzoylamino}-pyridin-2-yl ester,
Methyl-phenyl-carbamic acid 5-{4-[2-(4,4-dimethyl-piperidin-1-yl)-ethyl]-benzoylamino}-pyridin-2-yl ester,
Methyl-phenyl-carbamic acid 5-{4-[2-(2,6-dimethyl-piperidin-1-yl)-ethyl]-benzoylamino}-pyridin-2-yl ester, and
Methyl-phenyl-carbamic acid 5-{4-[2-(2,4,6-trimethyl-piperidin-1-yl)-ethyl]-benzoylamino}-pyridin-2-yl ester.

57. A pharmaceutical composition comprising a compound according to claim 1, or a pharmaceutically acceptable salt thereof together with a pharmaceutically acceptable carrier or diluent.

58. The composition according to claim 57, wherein said composition is in unit dosage form, comprising from about 0.05 to about 2000 mg, from about 0.1 to about 500 mg, or from about 1.0 to about 100 mg of said compound according to claim 1, or pharmaceutically acceptable salt thereof.

59. A pharmaceutical composition for use as a medicament for inhibiting the lipolytic activity of hormone-sensitive lipase against triacylglycerols, diacylglycerols, cholesterol acyl esters or steroid acyl esters, said composition comprising a compound according to claim 1, or a pharmaceutically acceptable salt thereof together with a pharmaceutically acceptable carrier or diluent.

60. A pharmaceutical composition according to claim 57, which is for oral administration.

61. A pharmaceutical composition according to claim 57, for nasal, transdermal, pulmonal, or parenteral administration.

62. A method of treating a disorder of a patient where modulation of the activity of hormone-sensitive lipase is desired, the method comprising administering to a subject in need thereof a therapeutically effective amount of a compound according to claim 1, or a pharmaceutically acceptable salt thereof.

63. A method of treating a disorder of a patient where lowering of the activity of hormone-sensitive lipase is desired, the method comprising administering to a subject in need thereof a therapeutically effective amount of a compound according to claim 1, or a pharmaceutically acceptable salt thereof.

64. The method according to claim 62, wherein said administration is carried out by the oral, nasal, transdermal, pulmonal, or parenteral route.

65. The method according to claim 62, wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

66. The method according to claim 62, wherein the therapeutically effective amount of the compound is from about 0.05 to about 2000 mg, from about 0.1 to about 500 mg, or from about 1.0 to about 100 mg of said compound per day.

67. The method according to claim 62, wherein a further antidiabetic, antiobesity, antihypertensive or appetite regulating drug is administered to the patient.

68. The method according to claim 62, wherein metformin is also administered to the patient.

69. A process for the preparation of a compound according to claim 1, or its pharmaceutically acceptable salt, comprising reacting the appropriate alcohol with the appropriate carbamoylating reagent in a solvent according to the reaction scheme P₁ 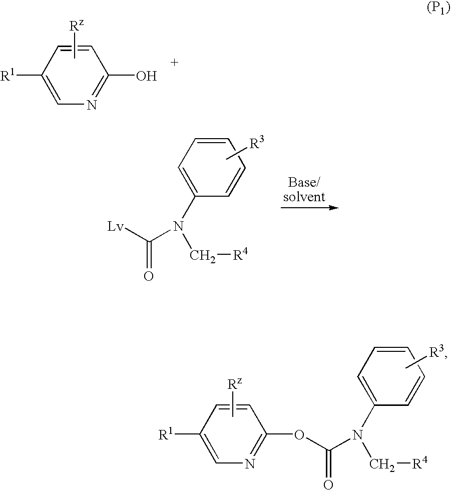
and isolating the disubstituted carbamate product.

70. The process according to claim 69, wherein said carbamoylating reagent 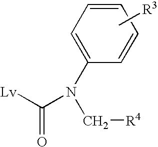
is selected from the group consisting of

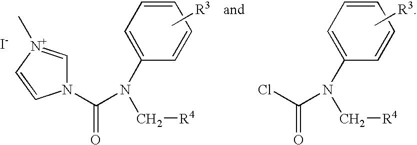

71. The process according to claim 69, wherein said solvent is selected from the group consisting of tetrahydrofurane, dimethylformamide and N-methylpyrolidone.

72. The process according to claim 69, wherein said base is selected from the group consisting of triethylamine, N,N-diisopropyl-N-ethylamine and DABCO.

73. A process for the preparation of a compound according to claim 1, said process comprising the treatment of the appropriate amine with the appropriate acylating reagent in a solvent and in the presence of a base according to the reaction scheme P₂ 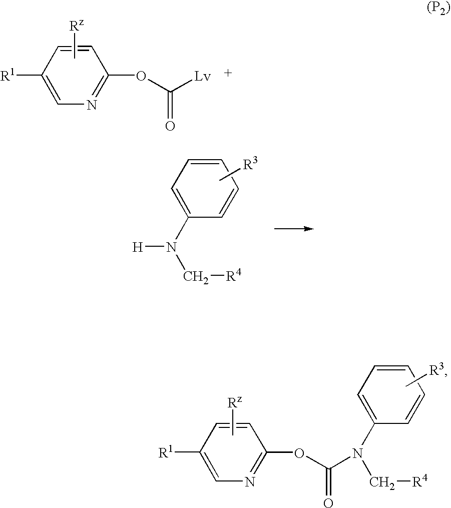
and isolating the disubstituted carbamate

74. The process according to claim 73, wherein Lv is Cl.

75. The process according to claim 73, wherein said solvent is selected from the group consisting of diethyl ether, tetrahydrofuran and dichloromethane.

76. The process according to claim 73, wherein said base is selected from the group consisting of trimethylamine, triethylamine, ethyl-diisopropyl-amine and 1,4-diazabicyclo[2.2.2]octane.

77. The process according to claim 73, wherein said base is present as a functionality in one or both of the substituents R³ and R⁴, thus forming a salt with the acid H-Lv.

78. A compound according to claim 1, wherein said compound is selected from the group consisting of:
Methyl-phenyl-carbamic acid 5-(4-dimethylaminomethyl-benzoylamino)-pyridin-2-yl ester,
Methyl-phenyl-carbamic acid 5-(4-diethylaminomethyl-benzoylamino)-pyridin-2-yl ester,
Methyl-phenyl-carbamic acid 5-(4-pyrrolidin-1-ylmethyl-benzoylamino)-pyridin-2-yl ester,
Methyl-phenyl-carbamic acid 5-(4-dipropylaminomethyl-benzoylamino)-pyridin-2-yl ester,
Methyl-phenyl-carbamic acid 5-(4-pyrrolidin-1-ylmethyl-benzoylamino)-pyridin-2-yl ester,
cis-Methyl-phenyl-carbamic acid 5-[4-(2,6-dimethyl-piperidin-1-ylmethyl)-benzoylamino]-pyridin-2-yl ester,
Methyl-phenyl-carbamic acid 5-[4-(4-oxo-piperidin-1-ylmethyl)-benzoylamino]-pyridin-2-yl ester,
cis-Methyl-phenyl-carbamic acid 5-[4-(2,6-dimethyl-morpholin-4-ylmethyl)-benzoylamino]-pyridin-2-yl ester,
Methyl-phenyl-carbamic acid 5-(4-thiomorpholin-4-ylmethyl-benzoylamino)-pyridin-2-yl ester,
Methyl-phenyl-carbamic acid 5-[3-(2-hydroxy-1,1-dimethyl-ethyl)-thioureido]-pyridin-2-yl ester,
Methyl-phenyl-carbamic acid 5-[3-(1-methyl-cyclopropyl)-thioureido]-pyridin-2-yl ester,
Methyl-phenyl-carbamic acid 5-[3-(1-methyl-cyclobutyl)-thioureido]-pyridin-2-yl ester,
Methyl-phenyl-carbamic acid 5-(4-imidazol-1-yl-benzoylamino)-pyridin-2-yl ester,
Methyl-phenyl-carbamic acid 5-(4-diethylamino-benzoylamino)-pyridin-2-yl ester,
Methyl-phenyl-carbamic acid 5-(4-[1,2,4]triazol-1-yl-benzoylamino)-pyridin-2-yl ester,
Methyl-phenyl-carbamic acid 5-(3,3-dipropyl-thioureido)-pyridin-2-yl ester,
Methyl-phenyl-carbamic acid 5-(3,3-dibutyl-thioureido)-pyridin-2-yl ester,
Methyl-phenyl-carbamic acid 5-[(piperidine-1-carbothioyl)-amino]-pyridin-2-yl ester,
Methyl-phenyl-carbamic acid 5-[(4-methyl-piperidine-1-carbothioyl)-amino]-pyridin-2-yl ester,
Methyl-phenyl-carbamic acid 5-[(4,4-dimethyl-piperidine-1-carbothioyl)-amino]-pyridin-2-yl ester,
(4-Bromo-phenyl)-methyl-carbamic acid 4,4-dimethyl-2,6-dioxo-3,4,5,6-tetrahydro-2H-[1,3′]bipyridinyl-6′-yl ester,
(4-Chloro-phenyl)-methyl-carbamic acid 4,4-dimethyl-2,6-dioxo-3,4,5,6-tetrahydro-2H-[1,3′]bipyridinyl-6′-yl ester,
(3,4-Dichloro-phenyl)-methyl-carbamic acid 4,4-dimethyl-2,6-dioxo-3,4,5,6-tetrahydro-2H-[1,3′]bipyridinyl-6′-yl ester,
(3-Chloro-phenyl)-methyl-carbamic acid 4,4-dimethyl-2,6-dioxo-3,4,5,6-tetrahydro-2H-[1,3′]bipyridinyl-6′-yl ester,
Methyl-p-tolyl-carbamic acid 4,4-dimethyl-2,6-dioxo-3,4,5,6-tetrahydro-2H-[1,3′]bipyridinyl-6′-yl ester,
(3-Fluoro-phenyl)-methyl-carbamic acid 4,4-dimethyl-2,6-dioxo-3,4,5,6-tetrahydro-2H-[1,3′]bipyridinyl-6′-yl ester,
Methyl-m-tolyl-carbamic acid 4,4-dimethyl-2,6-dioxo-3,4,5,6-tetrahydro-2H-[1,3′]bipyridinyl-6′-yl ester,
(4-Methoxy-phenyl)-methyl-carbamic acid 4,4-dimethyl-2,6-dioxo-3,4,5,6-tetrahydro-2H-[1,3′]bipyridinyl-6′-yl ester,
(3-Methoxy-phenyl)-methyl-carbamic acid 4,4-dimethyl-2,6-dioxo-3,4,5,6-tetrahydro-2H-[1,3′]bipyridinyl-6′-yl ester,
Methyl-(3-trifluoromethyl-phenyl)-carbamic acid 4,4-dimethyl-2,6-dioxo-3,4,5,6-tetrahydro-2H-[1,3′]bipyridinyl-6′-yl ester,
(3-Bromo-phenyl)-methyl-carbamic acid 4,4-dimethyl-2,6-dioxo-3,4,5,6-tetrahydro-2H-[1,3′]bipyridinyl-6′-yl ester,
(4-Fluoro-phenyl)-methyl-carbamic acid 4,4-dimethyl-2,6-dioxo-3,4,5,6-tetrahydro-2H-[1,3′]bipyridinyl-6′-yl ester, and
[4-(2-Hydroxy-ethyl)-phenyl]-methyl-carbamic acid 4,4-dimethyl-2,6-dioxo-3,4,5,6-tetrahydro-2H-[1,3′]bipyridinyl-6′-yl ester.
